(19) United States
(12) Patent Application Publication
(10) Pub. No.: US 2026/0365671 A1
(43) Pub. Date: Nov. 19, 2026

(54) HETEROCYCLIC COMPOUND

(71) Applicant: Takeda Pharmaceutical Company Limited, Osaka (JP)

(72) Inventors: Satoshi MIKAMI, Kanagawa (JP); Misaki HOMMA, Kanagawa (JP); Tomoaki HASUI, Kanagawa (JP); Yuta TANAKA TANAKA, Kanagawa (JP); Shinji MORIMOTO, Kanagawa (JP); Fumiaki KIKUCHI, Kanagawa (JP); Chunxiang WANG, Kanagawa (JP); Takafumi YUKAWA, Kanagawa (JP); Tomohiro OHASHI, Kanagawa (JP); Kosuke HIDAKA, Kanagawa (JP); Keisuke IMAMURA, Kanagawa (JP); Akinori TOITA, Kanagawa (JP); Shinji NAKAMURA, Kanagawa (JP); Masahiro ITO, Kanagawa (JP); Dilhumar UYGHUR, Kanagawa (JP); Masaki DAINI, Kanagawa (JP)

(21) Appl. No.: 19/102,384

(22) PCT Filed: Aug. 9, 2023
(86) PCT No.: PCT/IB2023/058071

§ 371 Date: Feb. 8, 2025

Related U.S. Application Data
(60) Provisional application No. 63/396,787, filed on Aug. 10, 2022.

(51) Int. Cl. C07D 513/04; A61K 9/20; A61K 9/48; A61K 31/4162; A61K 31/454

(52) U.S. Cl. CPC C07D 513/04; A61K 9/2018; A61K 9/2054; A61K 9/2059; A61K 9/4858; A61K 9/4866; A61K 31/4162; A61K 31/454

(57) ABSTRACT

The invention provides a compound of Formula (I), or a pharmaceutically acceptable salt thereof, or a solvate thereof, or a hydrate thereof: wherein X, Y, R¹, R², R³, R⁴, R⁵, and R⁶ are as defined in the specification, for treatment of a disease, disorder or condition associated with NLRP3, including a disease, disorder or condition associated with a heterozygous gain of function mutation in the NLRP3 gene such as cryopyrin-associated periodic syndrome (CAPS).

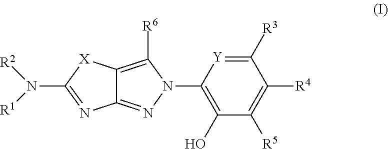

### FIELD OF THE INVENTION

This invention relates to heterocyclic compounds which are inhibitors of the NLRP3 inflammasome, to medicaments which contain them, and to their use to treat diseases, disorders and/or conditions associated with NLRP3, including neurodegenerative diseases, such as Parkinson's disease, Alzheimer's disease, Huntington's disease, amyotrophic lateral sclerosis and prion disease.

### BACKGROUND OF THE INVENTION

More than 1% of the world's population suffers from neurodegenerative diseases, including Parkinson's disease (PD), Alzheimer's disease (AD), Huntington's disease (HD), amyotrophic lateral sclerosis (ALS) and prion disease, all of which lack effective therapies. The incidence of neurodegenerative diseases is expected to double in the coming decades, especially affecting countries with an aging population. See I. Fernández-Cruz and E. Reynaud, “Proteasome Subunits Involved in Neurodegenerative Diseases,” Arch Med Res. 52(1):1-14 (2021).

One of the pathological hallmarks of neurodegenerative diseases is the aggregation of certain proteins into oligomers or fibrils. These conformational changes result in neurotoxicity, leading to inflammation and neurodegeneration. Although the clinical presentations of these diseases are heterogeneous, they often share common underlying mechanisms and pathophysiologies. See B. N. Dugger and D. W. Dickson, “Pathology of Neurodegenerative Diseases,” Cold Spring Harbor Perspect Biol 9(7): a028035 (2017). Indeed, systemic activation of the innate immune system, which is the first line of host defense against pathogens and tissue injury, and subsequent neuroinflammation play a key role in the onset and the progression of these diseases. See S. Amor, F. Puentes, D. Baker, et al., “Inflammation in neurodegenerative diseases,” Immunology 129(2):154-69 (2010). Neuroinflammation is a physiological response to exogenous and endogenous insults that target the central nervous system (CNS) and represents a protective response in the brain. However, excessive inflammatory responses are detrimental to the CNS. See L. I. Labzin, M. T. Heneka and E. Latz, “Innate Immunity and Neurodegeneration,” Annu Rev Med 69:437-449 (2018).

Microglia, which are myeloid cells of the CNS, play a major role during innate immune responses in the CNS. They express pattern recognition receptors (PRRs) which enable the host to recognize pathogen-associated molecular patterns (PAMPS) and host- or environment-derived danger-associated molecular patterns (DAMPS). See R. M. Ransohoff, M. A. Brown, “Innate immunity in the central nervous system,” J Clin Invest 122(4):1164-71 (2012). PRRs include Toll-like receptors, C-type lectin receptors, RIG-1 like receptors, and nucleotide-binding oligomerization domain-like receptors (NLRs). See P. Broz and V. M. Dixit, “Inflammasomes: mechanism of assembly, regulation and signaling,” Nat Rev Immunol 16(7):407-20 (2016). Engagement of PRRs activates a variety of inflammatory signaling pathways to eliminate infection and repair damaged tissue. The ongoing inflammation found in a variety of neurodegenerative diseases can be maintained by the key innate immune sensor for danger signals, the inflammasomes. There are several different inflammasomes, all defined by the PRRs they contain. Among the PRRs from the NLR family, the NLRs—NLRP1, NLRP3, NLRC4—and two other PRRs—Pyrin and AIM2—are known to form inflammasomes. See D. Zheng, T. Liwinski and E. Elinav, “Inflammasome activation and regulation: toward a better understanding of complex mechanisms,” Cell Discov 6:36 (2020).

The NLRP3 (nucleotide-binding domain (NOD), leucine-rich repeats-containing domain (LRR), and pyrin domain-containing 3) inflammasome has been the subject of intense interest in the past decade. See N. Kelley, D. Jeltema, Y. Duan, et al., “The NLRP3 Inflammasome: An Overview of Mechanisms of Activation and Regulation,” Int J Mol Sci 20(13):3328 (2019). The NLRP3 inflammasome consists of three main components: a pattern recognition receptor (PRR) protein, NLRP3; an apoptosis-associated speck-like protein (ASC) containing a caspase activation and recruitment domain (CARD), which functions as a central adaptor protein; and an inflammatory caspase, caspase-1. See Kelley et al. (2019). NLRP3 is comprised of three domains: an amino-terminal pyrin domain (PYD); a central NACHT domain, having ATPase activity that is vital for NLRP3 self-association and oligomerization; and a carboxy-terminal LRR domain. See Broz and Dixit (2016).

The activation of NLRP3 inflammasome involves a two-step process. A first “priming” signal is generated by the detection of PAMPs or DAMPs via TLRs. This priming signal results in NF-κB-dependent transcriptional upregulation of NLRP3 and pro-IL-1, but also controls post-translational modifications of NLRP3. See J. Yang, Z. Liu and T. S. Xiao, “Post-translational regulation of inflammasomes,” Cell Mol Immunol 14(1):65-79 (2017). The initial trigger is followed by a second “activation” signal (β-amyloid, α-synuclein and other proteinaceous insults, ATP, crystals, nucleic acids, toxins) that induces conformational change of the various inflammasome components to subsequently assemble and nucleate the oligomerization of monomeric NLRP3, leading to the formation and activation of the NLRP3 inflammasome. See A. Lu, V. G Magupalli, J. Ruan, et al., “Unified polymerization mechanism for the assembly of ASC-dependent inflammasomes,” Cell 156(6):1193-1206 (2014). This large multimeric protein acts via caspase-1 dependent proteolytic cleavage of several proteins, including pro-interleukin (pro-IL)-18 and pro-IL-1β to their mature inflammatory cytokines, IL-18 and IL-1β. See Kelley et al. (2019). Caspase-1 can also cleave gasdermin D (GSDMD), which facilitates GSDMD's insertion into cellular membranes to form pores, thus initiating a specific kind of cell death called pyroptosis that releases the soluble intracellular fraction which fuels the inflammatory response. See S. L. Fink and B. T Cookson, “Caspase-1-dependent pore formation during pyroptosis leads to osmotic lysis of infected host macrophages,” Cell Microbiol 8(11):1812-25 (2006).

Besides this “canonical” NLRP3 inflammasome activation pathway, a “noncanonical” NLRP3 activation pathway has been described in the literature. The noncanonical pathway involves the activation of caspase-4/5 (or its mouse ortholog caspase-11) by cytosolic LPS, the induction of pyroptosis through the cleavage of GSDMD, and the release of high mobility group box 1 protein (HMGB1), resulting in the production of IL-1β. See M. Lamkanfi and V. M. Dixit, “Mechanisms and functions of inflammasomes,” Cell 157(5):1013-22 (2014); F. Shi, Y. Yang, M. Kouadir M, et al., “Inhibition of phagocytosis and lysosomal acidification suppresses neurotoxic prion peptide-induced NALP3 inflammasome activation in BV2 microglia,” J Neuroimmunol 260(1-2):121-5 (2013). In both pathways, the activation of NLRP3 inflammasome results in the generation of the biologically active form of pro-inflammatory cytokines IL-1β and IL-18 that initiate inflammatory signaling cascades, contributing to neuroinflammation, neuronal injury and cell death. See S. M Allan, P. J. Tyrrell and N. J. Rothwell, “Interleukin-1 and neuronal injury,” Nat Rev Immunol, 5(8):629-40 (2005); A. Alboni, D. Cervia, S. Sugama, et al., “Interleukin 18 in the CNS,” J Neuroinflammation, 7:9 (2010).

Heterozygous gain of function mutations in the NLRP3 gene have been associated with the development of an autoinflammatory condition called cryopyrin-associated periodic syndromes (CAPS). See L. M. Booshehri and H. M. Hoffman, “CAPS and NLRP3,” J Clin Immunol 39(3):277-286 (2019). This is a rare inherited autoinflammatory disorder characterized by systemic, cutaneous, musculoskeletal and central nervous system inflammation, and is estimated to affect about 1 to 3 individuals per million people worldwide. See L. Cuisset, I. Jeru, B. Dumont, et al., “Mutations in the autoinflammatory cryopyrin-associated periodic syndrome gene: epidemiological study and lessons from eight years of genetic analysis in France,” Ann Rheum Dis 70(3):495-9 (2011); Erratum in: Ann Rheum Dis 71(7):1264 (2012). Clinicians classify CAPS disorders based on the severity of symptoms. The most severe form of CAPS is known as neonatal-onset multisystem inflammatory disease (NOMID/CINCA). An intermediate form of CAPS is called Muckle-Wells syndrome (MWS). The familial cold autoinflammatory syndrome (FCAS) is a milder form of CAPS, which is triggered by low temperatures. See Booshehri and Hoffman (2019). Current anti-IL-1 therapies (anakinra, rilonacept, canakinumab) have proven successful in treating CAPS, but clinical experience over the last decade has shown that some CAPS patients are less responsive over time and require higher or more frequent dosing or switching of therapies. See R. Caorsi, L. Lepore, F. Zulian, et al., “The schedule of administration of canakinumab in cryopyrin associated periodic syndrome is driven by the phenotype severity rather than the age,” Arthritis Res Ther 15(1): R³³ (2013); S. Urien, C. Bardin, B. Bader-Meunier, et al., “Anakinra pharmacokinetics in children and adolescents with systemic-onset juvenile idiopathic arthritis and autoinflammatory syndromes,” BMC Pharmacol Toxicol 14:40 (2013). Several small molecule inhibitors have recently been reported that block the NLRP3 inflammasome pathways. These include the prototype NLRP3 inhibitor MCC-950. See R. C. Coll, J. R. Hill, C. J. Day, et al., “MCC950 directly targets the NLRP3 ATP-hydrolysis motif for inflammasome inhibition,” Nat Chem Biol 15(6):556-559 (2019); R. C. Coll, A. A. Robertson, J. J. Chae, et al., “A small-molecule inhibitor of the NLRP3 inflammasome for the treatment of inflammatory diseases,” Nat Med 21(3):248-55 (2015). Other NLRP3 inhibitors include Bay 11-7082, CY-09, oridonin, tranilast, INF-39, glyburide and JC-124. See W. Jiang, M. Li, F. He, et al., “Inhibition of NLRP3 inflammasome attenuates spinal cord injury-induced lung injury in mice,” J Cell Physiol 234(5):6012-6022 (2019). MCC-950 has been used in many studies as a pharmacological tool to demonstrate NLRP3 inflammasome as a viable drug target to development therapeutics for human diseases. See S. E. Corcoran, R. Halai and M. A. Cooper, “Pharmacological Inhibition of the Nod-Like Receptor Family Pyrin Domain Containing 3 Inflammasome with MCC950,” Pharmacol Rev 73(3):968-1000 (2021).

Inhibitors of the NLRP3 inflammasome pathways are expected to be useful for treating neurodegenerative diseases, including Parkinson's disease, Alzheimer's disease, Huntington's disease, amyotrophic lateral sclerosis and prion disease, and for treating CAPS disorders associated with heterozygous gain of function mutations in the NLRP3 gene.

### SUMMARY OF THE INVENTION

This invention provides heterocyclic compounds, or pharmaceutically acceptable salts thereof, or solvates thereof, or hydrates thereof. This invention also provides medicaments that contain pyrazolothiazole/pyrazoloimidazole derivatives and provides for their use to treat diseases, disorders and/or conditions associated with NLRP3, including Parkinson's disease, Alzheimer's disease, Huntington's disease, amyotrophic lateral sclerosis and prion disease, and other neurodegenerative disorders.

One aspect of the invention provides [1] a compound of Formula (I), or a pharmaceutically acceptable salt thereof:

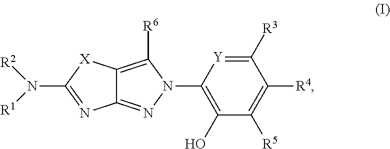

wherein
X is S or NR⁷;
Y is CR⁸ or N;
R¹ is an optionally substituted 4-membered heterocyclic group or 5- or 6-membered heterocyclic group, an optionally substituted C₁₋₆ alkyl group, or an optionally substituted C₃₋₁₀ cycloalkyl group;
R² is a hydrogen atom, or an optionally substituted C₁₋₃ alkyl group;
R³, R⁴ and R⁵ are each independently a hydrogen atom, a halogen atom, an optionally substituted C₁₋₆ alkyl group, an optionally substituted C₃₋₈ cycloalkyl group, or an optionally substituted C₁₋₆ alkoxy group;
R⁶ is a hydrogen atom, or an optionally substituted C₁₋₆ alkyl group;
R⁷ is a hydrogen atom, or an optionally substituted C₁₋₆ alkyl group; and
R⁸ is a hydrogen atom, a halogen atom, an optionally substituted C₁₋₆ alkyl group, an optionally substituted C₃₋₈ cycloalkyl, or an optionally substituted C₁₋₆ alkoxy group.

Some aspect of the invention provides [2] a compound of Formula (I), or a pharmaceutically acceptable salt thereof as defined in the above [1], wherein
X is S or NR⁷;
Y is CR⁸ or N;
R¹ is
(1) a 4-membered heterocyclic group or 5- or 6-membered heterocyclic group, wherein the heterocyclic groups are optionally substituted by 1 to 3 substituents selected from
   (a) a halogen atom,
   (b) an oxo group,
   (c) a C₁₋₆ alkyl group optionally substituted by 1 to 3 substituents selected from
      (i) a halogen atom, and
      (ii) a C₁₋₆ alkoxy group,
   (d) a C₃₋₈ cycloalkyl group,
   (e) a hydroxy group,
   (f) a C₁₋₆ alkoxy group,
   (g) a C₁₋₆ alkyl-carbonyl group optionally substituted by 1 to 3 substituents selected from
      (i) a halogen atom,
      (ii) a hydroxy group,
      (iii) a C₁₋₆ alkoxy group, and
      (iv) a C₃₋₈ cycloalkyl group,
   (h) a C₃₋₈ cycloalkyl-carbonyl group optionally substituted by 1 to 3 substituents selected from
      (i) a halogen atom,
      (ii) a cyano group, and
      (iii) a hydroxy group,
   (i) a C₁₋₆ alkoxy-carbonyl group optionally substituted by 1 to 3 halogen atoms,
   (j) a C₃₋₈ cycloalkoxy-carbonyl group,
   (k) a C₇₋₁₆ aralkyloxy-carbonyl group,
   (l) a mono- or di-C₁₋₆ alkyl-carbamoyl group,
   (m) a C₁₋₆ alkylsulfonyl group,
   (n) a C₃₋₈ cycloalkylsulfonyl group, and
   (o) a 3- to 8-membered non-aromatic heterocyclic group,
(2) a C₁₋₆ alkyl group optionally substituted by 1 to 3 substituents selected from
   (a) a hydroxy group,
   (b) a C₁₋₆ alkoxy group optionally substituted by 1 to 3 halogen atoms,
   (c) a C₃₋₈ cycloalkyl group optionally substituted by 1 to 3 halogen atoms,
   (d) a mono- or di-C₁₋₆ alkylamino group,
   (e) a C₁₋₆ alkyl-carbonylamino group,
   (f) a 3- to 8-membered non-aromatic heterocyclic group optionally substituted by a hydroxy group,
   (g) a halogen atom, and
   (h) a 5- to 14-membered aromatic heterocyclyl group, or
(3) a C₃₋₁₀ cycloalkyl group (the C₃₋₁₀ cycloalkyl group may be a spiro ring group or a bridged spiro ring group) optionally substituted by 1 to 3 substituents selected from
   (a) a hydroxy group,
   (b) a C₁₋₆ alkyl group optionally substituted by 1 to 3 halogen atoms, and
   (c) a C₁₋₆ alkoxy group optionally substituted by 1 to 3 halogen atoms;
R² is
(1) a hydrogen atom, or
(2) a C₁₋₃ alkyl group optionally substituted by 1 to 3 C₁₋₆ alkoxy groups;
R³ is a hydrogen atom;
R⁴ is
(1) a hydrogen atom,
(2) a halogen atom,
(3) a C₁₋₆ alkyl group optionally substituted by 1 to 3 halogen atoms, or
(4) a C₁₋₆ alkoxy group optionally substituted by 1 to 3 halogen atoms;
R⁵ is a hydrogen atom;
R⁶ is a hydrogen atom or a C₁₋₆ alkyl group;
R⁷ is a C₁₋₆ alkyl group; and
R⁸ is
(1) a hydrogen atom,
(2) a halogen atom,
(3) a C₁₋₆ alkyl group optionally substituted by 1 to 3 substituents selected from (i) halogen atoms and (ii) a C₁₋₆ alkoxy group optionally substituted by 1 to 3 halogen atoms,
(4) a C₃₋₈ cycloalkyl group optionally substituted by 1 to 3 halogen atoms, or
(5) a C₁₋₆ alkoxy group optionally substituted by 1 to 3 halogen atoms.

Some aspect of the invention provides [3] a compound of Formula (I), or a pharmaceutically acceptable salt thereof, as defined in the above [1] or [2], wherein R² is a hydrogen atom.

Some aspect of the invention provides [4] a compound of Formula (I), or a pharmaceutically acceptable salt thereof, as defined in the above [1] to [3], wherein X is NR⁷.

Some aspect of the invention provides [5] a compound of Formula (I), or a pharmaceutically acceptable salt thereof, as defined in any one of the above [1] to [3], wherein X is NR⁷ and R⁷ is a C₁₋₆ alkyl group.

Some aspect of the invention provides [6] a compound of Formula (I), or a pharmaceutically acceptable salt thereof, as defined in any one of the above [1] to [3], wherein X is S.

Some aspect of the invention provides [7] a compound of Formula (I), or a pharmaceutically acceptable salt thereof, as defined in any one of the above [1] to [6], wherein R¹ is
(1) a 4-membered heterocyclic group or 5- or 6-membered heterocyclic group, wherein the heterocyclic groups are optionally substituted by 1 to 3 substituents selected from
   (a) a halogen atom,
   (b) an oxo group,
   (c) a C₁₋₆ alkyl group optionally substituted by 1 to 3 substituents selected from
      (i) a halogen atom, and
      (ii) a C₁₋₆ alkoxy group,
   (d) a C₃₋₈ cycloalkyl group,
   (e) a hydroxy group,
   (f) a C₁₋₆ alkoxy group,
   (g) a C₁₋₆ alkyl-carbonyl group optionally substituted by 1 to 3 substituents selected from
      (i) a halogen atom,
      (ii) a hydroxy group,
      (iii) a C₁₋₆ alkoxy group, and
      (iv) a C₃₋₈ cycloalkyl group,
   (h) a C₃₋₈ cycloalkyl-carbonyl group optionally substituted by 1 to 3 substituents selected from
      (i) a halogen atom,
      (ii) a cyano group, and
      (iii) a hydroxy group,
   (i) a C₁₋₆ alkoxy-carbonyl group optionally substituted by 1 to 3 halogen atoms,
   (j) a C₃₋₈ cycloalkoxy-carbonyl group
   (k) a C₇₋₁₆ aralkyloxy-carbonyl group,
   (l) a mono- or di-C₁₋₆ alkyl-carbamoyl group,
   (m) a C₁₋₆ alkylsulfonyl group,
   (n) a C₃₋₈ cycloalkylsulfonyl group, and
   (o) a 3- to 8-membered non-aromatic heterocyclic group,
(2) a C₁₋₆ alkyl group optionally substituted by 1 to 3 substituents selected from
   (a) a hydroxy group,
   (b) a C₁₋₆ alkoxy group optionally substituted by 1 to 3 halogen atoms,
   (c) a C₃₋₈ cycloalkyl group optionally substituted by 1 to 3 halogen atoms,
   (d) a mono- or di-C₁₋₆ alkylamino group,
   (e) a C₁₋₆ alkyl-carbonylamino group,
   (f) a 3- to 8-membered non-aromatic heterocyclic group optionally substituted by a hydroxy group,
   (g) a halogen atom, and
   (h) a 5- to 14-membered aromatic heterocyclyl group, or
(3) a C₃₋₁₀ cycloalkyl group (the C₃₋₁₀ cycloalkyl group may be a spiro ring group or a bridged spiro ring group) optionally substituted by 1 to 3 substituents selected from
   (a) a hydroxy group,
   (b) a C₁₋₆ alkyl group optionally substituted by a to 3 halogen atoms, and
   (c) a C₁₋₆ alkoxy group optionally substituted by a to 3 halogen atoms.

Some aspect of the invention provides [8] a compound of Formula (I), or a pharmaceutically acceptable salt thereof, as defined in any one of the above [1] to [6], wherein R¹ is a 4-membered heterocyclic group or 5- or 6-membered non-aromatic heterocyclic group, wherein the heterocyclic groups are optionally substituted by 1 to 3 substituents selected from
(a) a halogen atom,
(b) an oxo group,
(c) a C₁₋₆ alkyl group optionally substituted by 1 to 3 substituents selected from
   (i) a halogen atom, and
   (ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
   (i) a halogen atom,
   (ii) a hydroxy group,
   (iii) a C₁₋₆ alkoxy group, and
   (iv) a C₃₋₈ cycloalkyl group,
(h) a C₃₋₈ cycloalkyl-carbonyl group optionally substituted by 1 to 3 substituents selected from
   (i) a halogen atom,
   (ii) a cyano group, and
   (iii) a hydroxy group,
(i) a C₁₋₆ alkoxy-carbonyl group optionally substituted by 1 to 3 halogen atoms,
(j) a C₃₋₈ cycloalkoxy-carbonyl group
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group.

Some aspect of the invention provides [9] a compound of Formula (I), or a pharmaceutically acceptable salt thereof, as defined in any one of the above [1] to [6], wherein R¹ is a 4-membered heterocyclic group or 5- or 6-membered non-aromatic heterocyclic group selected from pyrrolidinyl, piperidyl, oxetanyl, tetrahydrofuryl and tetrahydropyranyl, each of which is optionally substituted by 1 to 3 substituents selected from
(a) a halogen atom,
(b) an oxo group,
(c) a C₁₋₆ alkyl group optionally substituted by 1 to 3 substituents selected from
   (i) a halogen atom, and
   (ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
   (i) a halogen atom,
   (ii) a hydroxy group,
   (iii) a C₁₋₆ alkoxy group, and
   (iv) a C₃₋₈ cycloalkyl group,
(h) a C₃₋₈ cycloalkyl-carbonyl group optionally substituted by 1 to 3 substituents selected from
   (i) a halogen atom,
   (ii) a cyano group, and
   (iii) a hydroxy group,
(i) a C₁₋₆ alkoxy-carbonyl group optionally substituted by 1 to 3 halogen atoms,
(j) a C₃₋₈ cycloalkoxy-carbonyl group,
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group.

Some aspect of the invention provides [10] a compound of Formula (I), or a pharmaceutically acceptable salt thereof, as defined in any one of the above [1] to [9], wherein Y is CR⁸ wherein R⁸ is
(1) a hydrogen atom,
(2) a halogen atom,
(3) a C₁₋₆ alkyl group optionally substituted by 1 to 3 substituents selected from (i) halogen atoms and (ii) a C₁₋₆ alkoxy group optionally substituted by 1 to 3 halogen atoms,
(4) a C₃₋₈ cycloalkyl group optionally substituted by 1 to 3 halogen atoms, or
(5) a C₁₋₆ alkoxy group optionally substituted by 1 to 3 halogen atoms.

Some aspect of the invention provides [11] a compound of Formula (I), or a pharmaceutically acceptable salt thereof, as defined in any one of the above [1], [2], [4] to [6] and [10], wherein
R¹ is
(1) a 4-membered heterocyclic group or 5- or 6-membered heterocyclic group, wherein the heterocyclic groups are optionally substituted by 1 to 3 substituents selected from
   (a) a halogen atom,
   (b) an oxo group,
   (c) a C₁₋₆ alkyl group optionally substituted by 1 to 3 substituents selected from
      (i) a halogen atom, and
      (ii) a C₁₋₆ alkoxy group,
   (d) a C₃₋₈ cycloalkyl group,
   (e) a hydroxy group,
   (f) a C₁₋₆ alkoxy group,
   (g) a C₁₋₆ alkyl-carbonyl group optionally substituted by 1 to 3 substituents selected from
      (i) a halogen atom,
      (ii) a hydroxy group,
      (iii) a C₁₋₆ alkoxy group, and
      (iv) a C₃₋₈ cycloalkyl group,
   (h) a C₃₋₈ cycloalkyl-carbonyl group optionally substituted by 1 to 3 substituents selected from
      (i) a halogen atom,
      (ii) a cyano group, and
      (iii) a hydroxy group,
   (i) a C₁₋₆ alkoxy-carbonyl group optionally substituted by 1 to 3 halogen atoms,
   (j) a C₃₋₈ cycloalkoxy-carbonyl group,
   (k) a C₇₋₁₆ aralkyloxy-carbonyl group,
   (l) a mono- or di-C₁₋₆ alkyl-carbamoyl group,
   (m) a C₁₋₆ alkylsulfonyl group,
   (n) a C₃₋₈ cycloalkylsulfonyl group, and
   (o) a 3- to 8-membered non-aromatic heterocyclic group,
(2) a C₁₋₆ alkyl group optionally substituted by 1 to 3 substituents selected from
   (a) a hydroxy group,
   (b) a C₁₋₆ alkoxy group optionally substituted by 1 to 3 halogen atoms,
   (c) a C₃₋₈ cycloalkyl group optionally substituted by 1 to 3 halogen atoms,
   (d) a mono- or di-C₁₋₆ alkylamino group,
   (e) a C₁₋₆ alkyl-carbonylamino group,
   (f) a 3- to 8-membered non-aromatic heterocyclic group optionally substituted by a hydroxy group,
   (g) a halogen atom, and
   (h) a 5- to 14-membered aromatic heterocyclyl group, or
(3) a C₃₋₁₀ cycloalkyl group (the C₃₋₁₀ cycloalkyl group may be a spiro ring group or a bridged spiro ring group) optionally substituted by 1 to 3 substituents selected from
   (a) a hydroxy group,
   (b) a C₁₋₆ alkyl group optionally substituted by 1 to 3 halogen atoms, and
   (c) a C₁₋₆ alkoxy group optionally substituted by 1 to 3 halogen atoms; and
R² is a hydrogen atom.

Some aspect of the invention provides [12] a compound of Formula (I), or a pharmaceutically acceptable salt thereof, as defined in any one of the above [1], [2] and [10],
wherein
X is S;
R¹ is a 4-membered heterocyclic group or 5- or 6-membered non-aromatic heterocyclic group, wherein the heterocyclic groups are optionally substituted by 1 to 3 substituents selected from
   (a) a halogen atom,
   (b) an oxo group,
   (c) a C₁₋₆ alkyl group optionally substituted by 1 to 3 substituents selected from
      (i) a halogen atom, and
      (ii) a C₁₋₆ alkoxy group,
   (d) a C₃₋₈ cycloalkyl group,
   (e) a hydroxy group,
   (f) a C₁₋₆ alkoxy group,
   (g) a C₁₋₆ alkyl-carbonyl group optionally substituted by 1 to 3 substituents selected from
      (i) a halogen atom,
      (ii) a hydroxy group,
      (iii) a C₁₋₆ alkoxy group, and
      (iv) a C₃₋₈ cycloalkyl group,
   (h) a C₃₋₈ cycloalkyl-carbonyl group optionally substituted by 1 to 3 substituents selected from
      (i) a halogen atom,
      (ii) a cyano group, and
      (iii) a hydroxy group,
   (i) a C₁₋₆ alkoxy-carbonyl group optionally substituted by 1 to 3 halogen atoms,
   (j) a C₃₋₈ cycloalkoxy-carbonyl group,
   (k) a C₇₋₁₆ aralkyloxy-carbonyl group,
   (l) a mono- or di-C₁₋₆ alkyl-carbamoyl group,
   (m) a C₁₋₆ alkylsulfonyl group,
   (n) a C₃₋₈ cycloalkylsulfonyl group, and
   (o) a 3- to 8-membered non-aromatic heterocyclic group; and
R² is a hydrogen atom.

Some aspect of the invention provides [13] a compound of Formula (I), or a pharmaceutically acceptable salt thereof, as defined in any one of the above [1], [2] and [10], wherein
X is S;
R¹ is a 4-membered heterocyclic group or 5- or 6-membered non-aromatic heterocyclic group selected from pyrrolidinyl, piperidyl, oxetanyl, tetrahydrofuryl and tetrahydropyranyl, each of which is optionally substituted by 1 to 3 substituents selected from
   (a) a halogen atom,
   (b) an oxo group,
   (c) a C₁₋₆ alkyl group optionally substituted by 1 to 3 substituents selected from
      (i) a halogen atom, and
      (ii) a C₁₋₆ alkoxy group,
   (d) a C₃₋₈ cycloalkyl group,
   (e) a hydroxy group,
   (f) a C₁₋₆ alkoxy group,
   (g) a C₁₋₆ alkyl-carbonyl group optionally substituted by 1 to 3 substituents selected from
      (i) a halogen atom,
      (ii) a hydroxy group,
      (iii) a C₁₋₆ alkoxy group, and
      (iv) a C₃₋₈ cycloalkyl group,
   (h) a C₃₋₈ cycloalkyl-carbonyl group optionally substituted by 1 to 3 substituents selected from
      (i) a halogen atom,
      (ii) a cyano group, and
      (iii) a hydroxy group,
   (i) a C₁₋₆ alkoxy-carbonyl group optionally substituted by 1 to 3 halogen atoms,
   (j) a C₃₋₈ cycloalkoxy-carbonyl group,
   (k) a C₇₋₁₆ aralkyloxy-carbonyl group,
   (l) a mono- or di-C₁₋₆ alkyl-carbamoyl group,
   (m) a C₁₋₆ alkylsulfonyl group,
   (n) a C₃₋₈ cycloalkylsulfonyl group, and
   (o) a 3- to 8-membered non-aromatic heterocyclic group; and
R² is a hydrogen atom.

Some aspect of the invention provides [14] a compound of Formula (I), or a pharmaceutically acceptable salt thereof, as defined in any one of the above [1], [2], [7] to [9] and [11] to [13], wherein
X is S;
Y is CR⁸ wherein R⁸ is
(1) a hydrogen atom,
(2) a halogen atom,
(3) a C₁₋₆ alkyl group optionally substituted by 1 to 3 substituents selected from (i) halogen atoms and (ii) a C₁₋₆ alkoxy group optionally substituted by 1 to 3 halogen atoms,
(4) a C₃₋₈ cycloalkyl group optionally substituted by 1 to 3 halogen atoms, or
(5) a C₁₋₆ alkoxy group optionally substituted by 1 to 3 halogen atoms; and
R² is a hydrogen atom.

Some aspect of the invention provides [15] a compound of Formula (I), or a pharmaceutically acceptable salt thereof, as defined in any one of the above [1] to [14], wherein
R³ is a hydrogen atom;
R⁵ is a hydrogen atom; and
R⁶ is a hydrogen atom or a C₁₋₆ alkyl group.

Some aspect of the invention provides [16] a compound of Formula (I), or a pharmaceutically acceptable salt thereof, as defined in the above [1], [2] and [10], wherein
X is S;
R¹ is a 4-membered heterocyclic group or 5- or 6-membered non-aromatic heterocyclic group, wherein the heterocyclic groups are optionally substituted by 1 to 3 substituents selected from
   (a) a halogen atom,
   (b) an oxo group,
   (c) a C₁₋₆ alkyl group optionally substituted by 1 to 3 substituents selected from
      (i) a halogen atom, and
      (ii) a C₁₋₆ alkoxy group,
   (d) a C₃₋₈ cycloalkyl group,
   (e) a hydroxy group,
   (f) a C₁₋₆ alkoxy group,
   (g) a C₁₋₆ alkyl-carbonyl group optionally substituted by 1 to 3 substituents selected from
      (i) a halogen atom,
      (ii) a hydroxy group,
      (iii) a C₁₋₆ alkoxy group, and
      (iv) a C₃₋₈ cycloalkyl group,
   (h) a C₃₋₈ cycloalkyl-carbonyl group optionally substituted by 1 to 3 substituents selected from
      (i) a halogen atom,
      (ii) a cyano group, and
      (iii) a hydroxy group,
   (i) a C₁₋₆ alkoxy-carbonyl group optionally substituted by 1 to 3 halogen atoms,
   (j) a C₃₋₈ cycloalkoxy-carbonyl group,
   (k) a C₇₋₁₆ aralkyloxy-carbonyl group,
   (l) a mono- or di-C₁₋₆ alkyl-carbamoyl group,
   (m) a C₁₋₆ alkylsulfonyl group,
   (n) a C₃₋₈ cycloalkylsulfonyl group, and
   (o) a 3- to 8-membered non-aromatic heterocyclic group;
R² is a hydrogen atom;
R³ is a hydrogen atom;
R⁵ is a hydrogen atom; and
R⁶ is a hydrogen atom or a C₁₋₆ alkyl group.

Some aspect of the invention provides [17] a compound of Formula (I), or a pharmaceutically acceptable salt thereof, as defined in any one of the above [1], [2], [4] to [10] and [15], wherein R² is a C₁₋₃ alkyl group optionally substituted by 1 to 3 C₁₋₆ alkoxy groups.

Some aspect of the invention provides [18] a compound of Formula (I), or a pharmaceutically acceptable salt thereof, as defined in any one of the above [1], [2], [10] and [15], wherein
X is S;
R¹ is
(1) a 4-membered heterocyclic group or 5- or 6-membered heterocyclic group, wherein the heterocyclic groups are optionally substituted by 1 to 3 substituents selected from
   (a) a halogen atom,
   (b) an oxo group,
   (c) a C₁₋₆ alkyl group optionally substituted by 1 to 3 substituents selected from
      (i) a halogen atom, and
      (ii) a C₁₋₆ alkoxy group,
   (d) a C₃₋₈ cycloalkyl group,
   (e) a hydroxy group,
   (f) a C₁₋₆ alkoxy group,
   (g) a C₁₋₆ alkyl-carbonyl group optionally substituted by 1 to 3 substituents selected from
      (i) a halogen atom,
      (ii) a hydroxy group,
      (iii) a C₁₋₆ alkoxy group, and
      (iv) a C₃₋₈ cycloalkyl group,
   (h) a C₃₋₈ cycloalkyl-carbonyl group optionally substituted by 1 to 3 substituents selected from
      (i) a halogen atom,
      (ii) a cyano group, and
      (iii) a hydroxy group,
   (i) a C₁₋₆ alkoxy-carbonyl group optionally substituted by 1 to 3 halogen atoms,
   (j) a C₃₋₈ cycloalkoxy-carbonyl group,
   (k) a C₇₋₁₆ aralkyloxy-carbonyl group,
   (l) a mono- or di-C₁₋₆ alkyl-carbamoyl group,
   (m) a C₁₋₆ alkylsulfonyl group,
   (n) a C₃₋₈ cycloalkylsulfonyl group, and
   (o) a 3- to 8-membered non-aromatic heterocyclic group,
(2) a C₁₋₆ alkyl group optionally substituted by 1 to 3 substituents selected from
   (a) a hydroxy group,
   (b) a C₁₋₆ alkoxy group optionally substituted by 1 to 3 halogen atoms,
   (c) a C₃₋₈ cycloalkyl group optionally substituted by 1 to 3 halogen atoms,
   (d) a mono- or di-C₁₋₆ alkylamino group,
   (e) a C₁₋₆ alkyl-carbonylamino group,
   (f) a 3- to 8-membered non-aromatic heterocyclic group optionally substituted by a hydroxy group,
   (g) a halogen atom, and
   (h) a 5- to 14-membered aromatic heterocyclyl group, or
(3) a C₃₋₁₀ cycloalkyl group (the C₃₋₁₀ cycloalkyl group may be a spiro ring group or a bridged spiro ring group) optionally substituted by 1 to 3 substituents selected from
   (a) a hydroxy group,
   (b) a C₁₋₆ alkyl group optionally substituted by 1 to 3 halogen atoms, and
   (c) a C₁₋₆ alkoxy group optionally substituted by 1 to 3 halogen atoms; and
R² is a C₁₋₃ alkyl group optionally substituted by 1 to 3 C₁₋₆ alkoxy groups.

Some aspect of the invention provides [19] a compound of Formula (I), or a pharmaceutically acceptable salt thereof, as defined in the above [1] or [2], wherein
X is S;
Y is CR⁸ wherein R⁸ is a C₁₋₆ alkyl group;
R¹ is a 6-membered nitrogen-containing non-aromatic heterocyclic group substituted by 1 or 2 substituents selected from
   (a) a halogen atom, and
   (b) a C₁₋₆ alkyl group;
R² is a hydrogen atom;
R³ is a hydrogen atom;
R⁴ is a C₁₋₆ alkyl group;
R⁵ is a hydrogen atom; and
R⁶ is a hydrogen atom.

Some aspect of the invention provides [20] a compound of Formula (I), or a pharmaceutically acceptable salt thereof, as defined in the above [1] or [2], wherein
X is S;
Y is CR⁸ wherein R⁸ is a C₁₋₆ alkyl group;
R¹ is a piperidyl group substituted by 1 or 2 substituents selected from
   (a) a halogen atom, and
   (b) a C₁₋₂ alkyl group;
R² is a hydrogen atom;
R³ is a hydrogen atom;
R⁴ is a C₁₋₂ alkyl group;
R⁵ is a hydrogen atom; and
R⁶ is a hydrogen atom.

Another aspect of the invention provides [21] a compound of Formula (I) which is selected from the group of compounds described in the Examples (especially, 2-(5-{[(3R,5R)-5-fluoro-1-methylpiperidin-3-yl]amino}-2H-pyrazolo[3,4-d][1,3]thiazol-2-yl)-3,5-dimethylphenol, 3,5-dimethyl-2-(5-{[(3R)-1-methylpiperidin-3-yl]amino}-2H-pyrazolo[3,4-d][1,3]thiazol-2-yl)phenol, or 3-ethyl-2-(5-{[(3R,5R)-5-fluoro-1-methylpiperidin-3-yl]amino}-2H-pyrazolo[3,4-d][1,3]thiazol-2-yl)-5-methylphenol), pharmaceutically acceptable salts thereof.

A further aspect of the invention provides a compound of Formula (I), or a pharmaceutically acceptable salt thereof, or any one of the compounds, pharmaceutically acceptable salts, as defined in the above [1] to [21], for use as a medicament.

An additional aspect of the invention provides a medicament (also referred to as a pharmaceutical composition) which comprises a compound of Formula (I), or a pharmaceutically acceptable salt thereof, or any one of the compounds, pharmaceutically acceptable salts, as defined in the above [1] to [21]; and one or more pharmaceutically acceptable excipients for the treatment of a disease, disorder or condition associated with NLRP3, including a disease, disorder or condition associated with a heterozygous gain of function mutation in the NLRP3 gene such as cryopyrin-associated periodic syndrome (CAPS) including neonatal-onset multisystem inflammatory disease (NOMID/CINCA), Muckle-Wells syndrome (MWS), and familial cold autoinflammatory syndrome (FCAS). Since the compound of the present invention shows low toxicity (e.g., acute toxicity, chronic toxicity, genetic toxicity, reproductive toxicity, cardiotoxicity, carcinogenicity) and less side effects, it can be used as a prophylactic or therapeutic agent (collectively included in the term “medicament”), or diagnostic agent for various diseases in mammals (e.g., human, bovine, horse, dog, cat, monkey, mouse, rat).

Another aspect of the invention provides a compound of Formula (I), or a pharmaceutically acceptable salt thereof, or any one of the compounds, pharmaceutically acceptable salts, as defined in the above [1] to [21], for treatment of a disease, disorder or condition associated with NLRP3, including a disease, disorder or condition associated with a heterozygous gain of function mutation in the NLRP3 gene such as cryopyrin-associated periodic syndrome (CAPS) including neonatal-onset multisystem inflammatory disease (NOMID/CINCA), Muckle-Wells syndrome (MWS), and familial cold autoinflammatory syndrome (FCAS).

A further aspect of the invention provides a use of a compound of Formula (I), or a pharmaceutically acceptable salt thereof, or any one of the compounds, pharmaceutically acceptable salts, as defined in the above [1] to [21], for the manufacture of a medicament for the treatment of a disease, disorder or condition associated with NLRP3, including a disease, disorder or condition associated with a heterozygous gain of function mutation in the NLRP3 gene such as cryopyrin-associated periodic syndrome (CAPS) including neonatal-onset multisystem inflammatory disease (NOMID/CINCA), Muckle-Wells syndrome (MWS), and familial cold autoinflammatory syndrome (FCAS).

An additional aspect of the invention provides a method for treating a disease, disorder or condition associated with NLRP3, including a disease, disorder or condition associated with a heterozygous gain of function mutation in the NLRP3 gene such as cryopyrin-associated periodic syndrome (CAPS), the method comprising administering to the subject an effective amount of a compound of Formula (I), or a pharmaceutically acceptable salt thereof, or any one of the compounds, pharmaceutically acceptable salts, as defined in the above [1] to [21].

Another aspect of the invention provides a method for treating a cryopyrin-associated periodic syndrome (CAPS), including neonatal-onset multisystem inflammatory disease (NOMID/CINCA), Muckle-Wells syndrome (MWS), and familial cold autoinflammatory syndrome (FCAS), the method comprising administering to the subject an effective amount of a compound of Formula (I), or a pharmaceutically acceptable salt thereof, or any one of the compounds, pharmaceutically acceptable salts, as defined in the above [1] to [21].

A further aspect of the invention provides a method for treating a neurodegenerative disease, disorder or condition in a subject, the method comprising administering to the subject an effective amount of a compound of Formula (I), or a pharmaceutically acceptable salt thereof, or any one of the compounds, pharmaceutically acceptable salts, as defined in the above [1] to [21].

An additional aspect of the invention provides a method for treating Parkinson's disease, Alzheimer's disease, Huntington's disease, amyotrophic lateral sclerosis or prion disease in a subject, the method comprising administering to the subject an effective amount of a compound of Formula (I), a pharmaceutically acceptable salt thereof, or a solvate thereof, or a hydrate thereof, or any one of the compounds, pharmaceutically acceptable salts, solvates and hydrates as defined in the above [1] to [21].

Another aspect of the invention provides a combination comprising a compound of Formula (I), or a pharmaceutically acceptable salt thereof, or any one of the compounds, pharmaceutically acceptable salts, as defined in the above [1] to [21]; and at least one additional pharmacologically active agent (it is sometimes also referred as “pharmacologically active compound”) such as beta-secretase inhibitors, gamma-secretase inhibitors, HMG-CoA reductase inhibitors, nonsteroidal anti-inflammatory drugs, vitamin E, anti-amyloid antibodies, antidepressants, antipsychotics, anxiolytics, and anticonvulsants.

### DETAILED DESCRIPTION OF THE INVENTION

Unless otherwise indicated, this disclosure uses definitions provided below.

“Substituted,” when used in connection with a chemical substituent or moiety (e.g., a C₁₋₆ alkyl group), means that one or more hydrogen atoms of the substituent or moiety have been replaced with one or more non-hydrogen atoms or groups, provided valence requirements are met and a chemically stable compound results from the substitution.

“About” or “approximately,” when used in connection with a measurable numerical variable, refers to the indicated value of the variable and to all values of the variable that are within the experimental error of the indicated value or within ±10 percent of the indicated value, whichever is greater.

“Alkyl” refers to straight chain and branched saturated hydrocarbon groups, generally having a specified number of carbon atoms (e.g., C₁₋₃ alkyl refers to an alkyl group having 1 to 3 (i.e., 1, 2, or 3) carbon atoms, C₁₋₄ alkyl refers to an alkyl group having 1 to 4 (i.e., 1, 2, 3 or 4) carbon atoms, C₁₋₆ alkyl refers to an alkyl group having 1 to 6 (i.e., 1, 2, 3, 4, 5 or 6) carbon atoms, and so on). Examples of alkyl groups include methyl, ethyl, n-propyl, i-propyl (isopropyl), n-butyl, s-butyl (sec-butyl), i-butyl (isobutyl), t-butyl (tert-butyl), pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

“Alkanediyl” refers to divalent alkyl groups, where alkyl is defined above, and generally having a specified number of carbon atoms (e.g., C₁₋₄ alkanediyl refers to an alkanediyl group having 1 to 4 (i.e., 1, 2, 3 or 4) carbon atoms, C₁₋₆ alkanediyl refers to an alkanediyl group having 1 to 6 (i.e., 1, 2, 3, 4, 5 or 6) carbon atoms, and so on). Examples of alkanediyl groups include methylene, ethane-1,1-diyl, ethane-1,2-diyl, propane-1,3-diyl, propane-1,2-diyl, propane-1,1-diyl, propane-2,2-diyl, butane-1,4-diyl, butane-1,3-diyl, butane-1,2-diyl, butane-1,1-diyl, isobutane-1,3-diyl, isobutane-1,1-diyl, isobutane-1,2-diyl, and the like.

“Alkenyl” refers to straight chain and branched hydrocarbon groups having one or more carbon-carbon double bonds, and generally having a specified number of carbon atoms (e.g., C₂₋₆ alkenyl refers to an alkenyl group having 2 to 6 (i.e., 2, 3, 4, 5 or 6) carbon atoms, and so on). Examples of alkenyl groups include ethenyl, 1-propen-1-yl, 1-propen-2-yl, 2-propen-1-yl, 1-buten-1-yl, 1-buten-2-yl, 3-buten-1-yl, 3-buten-2-yl, 2-buten-1-yl, 2-buten-2-yl, 2-methyl-1-propen-1-yl, 2-methyl-2-propen-1-yl, 1,3-butadien-1-yl, 1,3-butadien-2-yl, and the like.

“Alkynyl” refers to straight chain or branched hydrocarbon groups having one or more triple carbon-carbon bonds, and generally having a specified number of carbon atoms (e.g., C₂₋₆ alkynyl refers to an alkynyl group having 2 to 6 (i.e., 2, 3, 4, 5 or 6) carbon atoms, and so on). Examples of alkynyl groups include ethynyl, 1-propyn-1-yl, 2-propyn-1-yl, 1-butyn-1-yl, 3-butyn-1-yl, 3-butyn-2-yl, 2-butyn-1-yl, and the like.

“Alkoxy” refers to straight chain and branched saturated hydrocarbon groups attached through an oxygen atom, generally having a specified number of carbon atoms (e.g., C₁₋₄ alkoxy refers to an alkoxy group having 1 to 4 (i.e., 1, 2, 3 or 4) carbon atoms, C₁₋₆ alkoxy refers to an alkoxy group having 1 to 6 (i.e., 1, 2, 3, 4, 5 or 6) carbon atoms, and so on). Examples of alkoxy groups include methoxy, ethoxy, n-propoxy, i-propoxy, n-butoxy, s-butoxy, i-butoxy, t-butoxy, pent-1-yloxy, pent-2-yloxy, pent-3-yloxy, 3-methylbut-1-yloxy, 3-methylbut-2-yloxy, 2-methylbut-2-yloxy, 2,2,2-trimethyleth-1-yloxy, n-hexoxy, and the like.

“Alkyl-carbonyl” and “alkylsulfonyl” refer to an alkyl group as defined above, which is attached, respectively, through a carbonyl (C(O)) group or a sulfonyl (SO₂) group, and generally having a specified number of carbon atoms (e.g., C₁₋₆ alkyl-carbonyl refers to an alkyl-carbonyl group having 1 to 6 (i.e., 1, 2, 3, 4, 5 or 6) carbon atoms, excluding the carbonyl moiety, C₁₋₆ alkylsulfonyl refers to an alkylsulfonyl group having 1 to 6 (i.e., 1, 2, 3, 4, 5 or 6) carbon atoms, and so on). Examples of alkyl-carbonyl groups include methylcarbonyl (acetyl), ethylcarbonyl, i-propylcarbonyl (propanoyl), n-propylcarbonyl, 2-methylpropanoyl, and the like. Examples of alkylsulfonyl groups include methylsulfonyl, ethylsulfonyl, i-propylsulfonyl, n-propylsulfonyl, and the like.

“Alkylamino” including mono- or di-alkylamino group refers to an alkyl group as defined above, which is attached through at least one amino group, and generally having a specified number of carbon atoms (e.g., C₁₋₆ alkylamino refers to a mono- or di-alkylamino group having 1 to 6 (i.e., 1, 2, 3, 4, 5 or 6) carbon atoms, and so on). Examples of mono- or di-alkylamino groups include methylamino, ethylamino, propylamino, isopropylamino, butylamino, dimethylamino, diethylamino, dipropylamino, dibutylamino, N-ethyl-N-methylamino, and the like.

“Alkyl-carbamoyl” including mono- or di-alkyl-carbamoyl group refers to an alkyl group as defined above, which is attached through a carbamoyl (CONH₂) group, and generally having a specified number of carbon atoms (e.g., C₁₋₆ alkyl-carbamoyl refers to a mono- or di-alkyl-carbamoyl group having 1 to 6 (i.e., 1, 2, 3, 4, 5 or 6) carbon atoms, excluding the carbamoyl moiety, and so on). Examples of mono- or di-alkyl-carbamoyl groups include methylcarbamoyl, ethylcarbamoyl, dimethylcarbamoyl, diethylcarbamoyl and N-ethyl-N-methylcarbamoyl, and the like.

“Alkyl-carbonylamino” refers to an alkyl-carbonyl as defined above, which is attached through an amino moiety, and generally having a specified number of carbon atoms (e.g., C₁₋₆ alkyl-carbonylamino refers to an alkyl-carbonylamino group having 1 to 6 (i.e., 1, 2, 3, 4, 5 or 6) carbon atoms, excluding the carbonyl moiety, and so on). Examples of C₁₋₆ alkyl-carbonylamino groups include methylcarbonylamino (acetylamino), ethylcarbonylamino, and the like.

“Alkoxy-carbonyl” refers to an alkoxy group as defined above, which is attached through a carbonyl (C(O)) group, and generally having a specified number of carbon atoms (e.g., C₁₋₆ alkoxy-carbonyl refers to an alkoxy-carbonyl group having 1 to 6 (i.e., 1, 2, 3, 4, 5 or 6) carbon atoms, excluding the carbonyl moiety, and so on). Examples of C₁₋₆ alkoxy-carbonyl groups include methoxycarbonyl, ethoxycarbonyl, and the like.

“Halo,” “halogen” and “halogeno” may be used interchangeably and refer to fluoro, chloro, bromo, and iodo.

“Haloalkyl,” “haloalkenyl,” and “haloalkynyl,” refer, respectively, to alkyl, alkenyl, and alkynyl groups substituted with one or more halogen atoms, where alkyl, alkenyl, and alkynyl are defined above, and generally having a specified number of carbon atoms. Examples of haloalkyl groups include fluoromethyl, difluoromethyl, trifluoromethyl, chloromethyl, dichloromethyl, trichloromethyl, 1-fluoroethyl, 1,1-difluoroethyl, 1-chloroethyl, 1,1-dichloroethyl, 1-fluoro-1-methylethyl, 1-chloro-1-methylethyl, and the like.

“Cycloalkyl” refers to saturated monocyclic, bicyclic hydrocarbon groups, and tricyclic hydrocarbon groups generally having a specified number of carbon atoms that comprise the ring or rings (e.g., C₃₋₈ cycloalkyl refers to a cycloalkyl group having 3 to 8 (i.e., 3, 4, 5, 6, 7 or 8) carbon atoms as ring members, and C₃₋₁₀ cycloalkyl refers to a cycloalkyl group having 3 to 10 (i.e., 3, 4, 5, 6, 7, 8, 9 or 10) carbon atoms as ring members). Bicyclic hydrocarbon groups may include isolated rings (two rings sharing no carbon atoms), spiro rings (two rings sharing one carbon atom), fused rings (two rings sharing two carbon atoms and the bond between the two common carbon atoms), and bridged rings (two rings sharing two carbon atoms, but not a common bond). Tricyclic hydrocarbon groups may include isolated rings (two rings sharing no carbon atoms), spiro rings (two rings sharing one carbon atom), fused rings (two rings of the tricyclic ring sharing two carbon atoms and the bond between the two common carbon atoms), and bridged rings (two rings of the tricyclic ring sharing two carbon atoms, but not a common bond). The cycloalkyl group may be attached through any ring atom unless such attachment would violate valence requirements, and where indicated, may optionally include one or more non-hydrogen substituents unless such substitution would violate valence requirements.

Examples of monocyclic cycloalkyl groups include cyclopropyl, cyclobutyl, cyclopentyl, cyclohexyl, and the like. Examples of fused bicyclic cycloalkyl groups include bicyclo[2.1.0]pentanyl (i.e., bicyclo[2.1.0]pentan-1-yl, bicyclo[2.1.0]pentan-2-yl, and bicyclo[2.1.0]pentan-5-yl), bicyclo[3.1.0]hexanyl, bicyclo[3.2.0]heptanyl, bicyclo[4.1.0]heptanyl, bicyclo[3.3.0]octanyl, bicyclo[4.2.0]octanyl, bicyclo[4.3.0]nonanyl, bicyclo[4.4.0]decanyl, and the like. Examples of bridged cycloalkyl groups include bicyclo[2.1.1]hexanyl, bicyclo[2.2.1]heptanyl, bicyclo[3.1.1]heptanyl, bicyclo[2.2.2]octanyl, bicyclo[3.2.1]octanyl, bicyclo[4.1.1]octanyl, bicyclo[3.3.1]nonanyl, bicyclo[4.2.1]nonanyl, bicyclo[3.3.2]decanyl, bicyclo[4.2.2]decanyl, bicyclo[4.3.1]decanyl, bicyclo[3.3.3]undecanyl, bicyclo[4.3.2]undecanyl, bicyclo[4.3.3]dodecanyl, adamantyl (tricyclo[3.3.1.1^{3,7}]decanyl) and the like. Examples of spiro cycloalkyl groups include spiro[3.3]heptanyl, spiro[2.4]heptanyl, spiro[3.4]octanyl, spiro[2.5]octanyl, spiro[3.5]nonanyl, and the like. Examples of isolated bicyclic cycloalkyl groups include those derived from bi(cyclobutane), cyclobutanecyclopentane, bi(cyclopentane), cyclobutanecyclohexane, cyclopentanecyclohexane, bi(cyclohexane), etc.

“Cycloalkanediyl” refers to divalent cycloalkyl groups, where cycloalkyl is defined above, and generally having a specified number of carbon atoms (e.g., C₃₋₈ cycloalkanediyl refers to a cycloalkanediyl group having 3 to 8 (i.e., 3, 4, 5, 6, 7 or 8) carbon atoms, and so on). Examples of cycloalkanediyl groups include cyclopropane-1,1-diyl, cyclopropane-1,2-diyl, cyclobutane-1,1-diyl, cyclobutane-1,2-diyl, and the like.

“Cycloalkylidene” refers to divalent monocyclic cycloalkyl groups, where cycloalkyl is defined above, which are attached through a single carbon atom of the group, and generally having a specified number of carbon atoms that comprise the ring (e.g., C₃₋₈ cycloalkylidene refers to a cycloalkylidene group having 3 to 8 (i.e., 3, 4, 5, 6, 7 or 8) carbon atoms as ring members). Examples of cycloalkylidene groups include cyclopropylidene, cyclobutylidene, cyclopentylidene, and cyclohexylidene.

“Cycloalkenyl” refers to partially unsaturated monocyclic and bicyclic hydrocarbon groups, generally having a specified number of carbon atoms that comprise the ring or rings (e.g., C₃₋₈ cycloalkenyl refers to a cycloalkenyl group having 3 to 8 (i.e., 3, 4, 5, 6, 7 or 8) carbon atoms, and so on). As with cycloalkyl groups, the bicyclic cycloalkenyl groups may include isolated, spiro, fused, or bridged rings. Similarly, the cycloalkenyl group may be attached through any ring atom, and where indicated, may optionally include one or more non-hydrogen substituents unless such attachment or substitution would violate valence requirements. Examples of cycloalkenyl groups include the partially unsaturated analogs of the cycloalkyl groups described above, such as cyclobutenyl (i.e., cyclobuten-1-yl and cyclobuten-3-yl), cyclopentenyl, cyclohexenyl, bicyclo[2.2.1]hept-2-enyl, and the like.

“Cycloalkyl-carbonyl” or “cycloalkylsulfonyl” refers to a cycloalkyl group as defined above, which is attached, respectively, through a carbonyl (C(O)) group or a sulfonyl (SO₂) group, and generally having a specified number of carbon atoms (e.g., C₃₋₈ cycloalkyl-carbonyl refers to a cycloalkyl-carbonyl group having 3 to 8 (i.e., 3, 4, 5, 6, 7 or 8) carbon atoms, excluding the carbonyl moiety, as ring members of cycloalkyl group, C₃₋₈ cycloalkylsulfonyl refers to a cycloalkylsulfonyl group having 3 to 8 (i.e., 3, 4, 5, 6, 7 or 8) carbon atoms as ring members of cycloalkyl group, and so on). Examples of cycloalkyl-carbonyl groups include cyclopropylcarbonyl cyclobutylcarbonyl, cyclopentylcarbonyl, and the like. Examples of cycloalkylsulfonyl groups include cyclopropylsulfonyl cyclobutylsulfonyl, cyclopentylsulfonyl, and the like.

“Aryl” refers to fully unsaturated monocyclic aromatic hydrocarbons and to polycyclic hydrocarbons having at least one aromatic ring, both monocyclic and polycyclic aryl groups generally having a specified number of carbon atoms that comprise their ring members (e.g., C₆₋₁₄ aryl refers to an aryl group having 6 to 14 carbon atoms as ring members, and so on). The group may be attached through any ring atom, and where indicated, may optionally include one or more non-hydrogen substituents unless such attachment or substitution would violate valence requirements. Examples of aryl groups include phenyl, biphenyl, cyclobutabenzenyl, indenyl, naphthyl, benzocycloheptanyl, biphenylenyl, fluorenyl, groups derived from cycloheptatriene cation, and the like.

“Acyl group” include a formyl group, a carboxy group, a carbamoyl group, a thiocarbamoyl group, a sulfino group, a sulfo group, a sulfamoyl group and a phosphono group, each optionally having “1 or 2 substituents selected from a C₁₋₆ alkyl group, a C₂₋₆ alkenyl group, a C₃₋₁₀ cycloalkyl group, a C₃₋₁₀ cycloalkenyl group, a C₆₋₁₄ aryl group, a C₇₋₁₆ aralkyl group, a 5- to 14-membered aromatic heterocyclic group and a 3- to 14-membered non-aromatic heterocyclic group, each of which optionally has 1 to 3 substituents selected from a halogen atom, an optionally halogenated C₁₋₆ alkoxy group, a hydroxy group, a nitro group, a cyano group, an amino group and a carbamoyl group”. Examples of the “acyl group” also include a hydrocarbon-sulfonyl group, a heterocyclylsulfonyl group, a hydrocarbon-sulfinyl group and a heterocyclylsulfinyl group. Here, the hydrocarbon-sulfonyl group means a hydrocarbon group-bonded sulfonyl group, the heterocyclylsulfonyl group means a heterocyclic group-bonded sulfonyl group, the hydrocarbon-sulfinyl group means a hydrocarbon group-bonded sulfinyl group and the heterocyclylsulfinyl group means a heterocyclic group-bonded sulfinyl group.

Preferable examples of the “acyl group” include a formyl group, a carboxy group, a C₁₋₆ alkyl-carbonyl group, a C₂₋₆ alkenyl-carbonyl group (e.g., crotonoyl), a C₃₋₁₀ cycloalkyl-carbonyl group (e.g., cyclobutanecarbonyl, cyclopentanecarbonyl, cyclohexanecarbonyl, cycloheptanecarbonyl), a C₃₋₁₀ cycloalkenyl-carbonyl group (e.g., 2-cyclohexenecarbonyl), a C₆₋₁₄ aryl-carbonyl group, a C₇₋₁₆ aralkyl-carbonyl group, a 5- to 14-membered aromatic heterocyclylcarbonyl group, a 3- to 14-membered non-aromatic heterocyclylcarbonyl group, a C₁₋₆ alkoxy-carbonyl group, a C₆₋₁₄ aryloxy-carbonyl group (e.g., phenyloxycarbonyl, naphthyloxycarbonyl), a C₇₋₁₆ aralkyloxy-carbonyl group (e.g., benzyloxycarbonyl, phenethyloxycarbonyl), a carbamoyl group, a mono- or di-C₁₋₆ alkyl-carbamoyl group, a mono- or di-C₂₋₆ alkenyl-carbamoyl group (e.g., diallylcarbamoyl), a mono- or di-C₃₋₁₀ cycloalkyl-carbamoyl group (e.g., cyclopropylcarbamoyl), a mono- or di-C₆₋₁₄ aryl-carbamoyl group (e.g., phenylcarbamoyl), a mono- or di-C₇₋₁₆ aralkyl-carbamoyl group, a 5- to 14-membered aromatic heterocyclylcarbamoyl group (e.g., pyridylcarbamoyl), a thiocarbamoyl group, a mono- or di-C₁₋₆ alkyl-thiocarbamoyl group (e.g., methylthiocarbamoyl, N-ethyl-N-methylthiocarbamoyl), a mono- or di-C₂₋₆ alkenyl-thiocarbamoyl group (e.g., diallylthiocarbamoyl), a mono- or di-C₃₋₁₀ cycloalkyl-thiocarbamoyl group (e.g., cyclopropylthiocarbamoyl, cyclohexylthiocarbamoyl), a mono- or di-C₆₋₁₄ aryl-thiocarbamoyl group (e.g., phenylthiocarbamoyl), a mono- or di-C₇₋₁₆ aralkyl-thiocarbamoyl group (e.g., benzylthiocarbamoyl, phenethylthiocarbamoyl), a 5- to 14-membered aromatic heterocyclylthiocarbamoyl group (e.g., pyridylthiocarbamoyl), a sulfino group, a C₁₋₆ alkylsulfinyl group (e.g., methylsulfinyl, ethylsulfinyl), a sulfo group, a C₁₋₆ alkylsulfonyl group, a C₆₋₁₄ arylsulfonyl group, a phosphono group and a mono- or di-C₁₋₆ alkylphosphono group (e.g., dimethylphosphono, diethylphosphono, diisopropylphosphono, dibutylphosphono).

“Aralkyl” refers to an alkyl group as defined above, wherein one of its hydrogens is substituted by an aryl group as defined above, and generally having a specified number of carbon atoms (e.g., C₇₋₁₆ aralkyl refers to an aralkyl group having 7 to 16 carbon atoms, and so on). Examples of aralkyl groups include benzyl, phenethyl, naphthylmethyl, phenylpropyl, and the like.

“Aralkyloxy” refers to a hydroxy group whose hydrogen is substituted by an aralkyl group as defined above, and generally having a specified number of carbon atoms (e.g., C₇₋₁₆ aralkyloxy refers to an aralkyloxy group having 7 to 16 carbon atoms, and so on). Examples of aralkyloxy groups include benzyloxy, phenethyloxy, naphthylmethoxy, phenylpropyloxy, and the like.

“Aralkyloxy-carbonyl” refers to an aralkyloxy group as defined above, which is attached through a carbonyl (C(O)) group, and generally having a specified number of carbon atoms (e.g., C₇₋₁₆ aralkyloxy-carbonyl refers to an aralkyloxy-carbonyl group having 7 to 16 carbon atoms, excluding the carbonyl moiety, and so on). Examples of aralkyloxy-carbonyl groups include benzyloxycarbonyl, phenethyloxycarbonyl, naphthylmethoxycarbonyl, phenylpropyloxycarbonyl, and the like.

“Arylene” refers to divalent aryl groups, where aryl is defined above, and generally having a specified number of carbon atoms that comprise their ring members (e.g., C₆₋₁₄ arylene refers to an arylene group having 6 to 14 carbon atoms as ring members, and so on). Examples of arylene groups include o-phenylene (i.e., benzene-1,2-diyl).

“Heterocycle”, “heterocyclic” and “heterocyclyl” may be used interchangeably and refer to saturated or partially unsaturated monocyclic or bicyclic groups having ring atoms composed of carbon atoms and one or more heteroatoms independently selected from nitrogen, oxygen, and sulfur. Both the monocyclic and bicyclic groups generally have a specified number of carbon atoms in their ring or rings (e.g., C₂₋₆ heterocyclyl refers to a heterocyclyl group having 2 to 6 (i.e., 2, 3, 4, 5 or 6) carbon atoms and, e.g., 1 to 4 (i.e., 1, 2, 3 or 4) heteroatoms, as ring members, while 4-membered heterocyclic group or 5- or 6-membered heterocyclic group refers to a heterocyclyl group having 4 to 6 atoms as ring members in total of carbon atoms and heteroatoms). As with bicyclic cycloalkyl groups, bicyclic heterocyclyl groups may include isolated rings, spiro rings, fused rings, and bridged rings. The heterocyclyl group may be attached through any ring atom, and where indicated, may optionally include one or more non-hydrogen substituents unless such attachment or substitution would violate valence requirements or result in a chemically unstable compound. Examples of heterocyclyl groups include oxiranyl, thiiranyl, aziridinyl (e.g., aziridin-1-yl and aziridin-2-yl), oxetanyl, thietanyl, azetidinyl, tetrahydrofuranyl, tetrahydrothienyl, pyrrolidinyl, tetrahydropyranyl, tetrahydrothiopyranyl, piperidinyl, 1,4-dioxanyl, 1,4-oxathianyl, morpholinyl, 1,4-dithianyl, piperazinyl, 1,4-azathianyl, oxepanyl, thiepanyl, azepanyl, 1,4-dioxepanyl, 1,4-oxathiepanyl, 1,4-oxaazepanyl, 1,4-dithiepanyl, 1,4-thiazepanyl, 1,4-diazepanyl, 3,4-dihydro-2H-pyranyl, 3,6-dihydro-2H-pyranyl, 2H-pyranyl, 1,2-dihydropyridinyl, 1,2,3,4-tetrahydropyridinyl, 1,2,5,6-tetrahydropyridinyl, 1,6-dihydropyrimidinyl, 1,2,3,4-tetrahydropyrimidinyl, and 1,2-dihydropyrazolo[1,5-d][1,2,4]triazinyl.

“Heterocycle-diyl” refers to heterocyclyl groups which are attached through two ring atoms of the group, where heterocyclyl is defined above. They generally have a specified number of carbon atoms in their ring or rings (e.g., C₂₋₆ heterocycle-diyl refers to a heterocycle-diyl group having 2 to 6 (i.e., 2, 3, 4, 5 or 6) carbon atoms and, e.g., 1 to 4 (i.e., 1, 2, 3 or 4) heteroatoms, as ring members). Examples of heterocycle-diyl groups include the multivalent analogs of the heterocycle groups described above, such as morpholine-3,4-diyl, pyrrolidine-1,2-diyl, 1-pyrrolidinyl-2-ylidene, 1-pyridinyl-2-ylidene, 1-(4H)-pyrazolyl-5-ylidene, 1-(3H)-imidazolyl-2-ylidene, 3-oxazolyl-2-ylidene, 1-piperidinyl-2-ylidene, 1-piperazinyl-6-ylidene, and the like.

“Heteroaromatic”, “aromatic heterocyclyl/heterocyclic” and “heteroaryl” may be used interchangeably and refer to unsaturated monocyclic aromatic groups and to polycyclic groups having at least one aromatic ring, each of the groups having ring atoms composed of carbon atoms and one or more heteroatoms independently selected from nitrogen, oxygen, and sulfur. Both the monocyclic and polycyclic groups generally have a specified number of carbon atoms as ring members (e.g., C₁₋₉ heteroaryl refers to a heteroaryl group having 1 to 9 (i.e., 1, 2, 3, 4, 5, 6, 7, 8 or 9) carbon atoms and, e.g., 1 to 4 (i.e., 1, 2, 3 or 4) heteroatoms, as ring members) and may include any bicyclic group in which any of the above-listed monocyclic heterocycles are fused to a benzene ring. And the monocyclic and polycyclic groups are 5- to 14-membered aromatic heterocyclyl groups. The heteroaryl group may be attached through any ring atom (or ring atoms for fused rings), and where indicated, may optionally include one or more non-hydrogen substituents unless such attachment or substitution would violate valence requirements or result in a chemically unstable compound. Examples of heteroaryl groups include monocyclic groups such as pyrrolyl (e.g., pyrrol-1-yl, pyrrol-2-yl, and pyrrol-3-yl), furyl, thienyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, and pyrazinyl.

Examples of heteroaryl groups also include bicyclic groups such as benzofuranyl, isobenzofuranyl, benzothienyl, benzo[c]thienyl, 1H-indolyl, 3H-indolyl, isoindolyl, 1H-isoindolyl, indolinyl, isoindolinyl, benzimidazolyl, 1H-indazolyl, 2H-indazolyl, benzotriazolyl, 1H-pyrrolo[2,3-b]pyridinyl, 1H-pyrrolo[2,3-c]pyridinyl, 1H-pyrrolo[3,2-c]pyridinyl, 1H-pyrrolo[3,2-b]pyridinyl, 3H-imidazo[4,5-b]pyridinyl, 3H-imidazo[4,5-c]pyridinyl, 1H-pyrazolo[4,3-b]pyridinyl, 1H-pyrazolo[4,3-c]pyridinyl, 1H-pyrazolo[3,4-c]pyridinyl, 1H-pyrazolo[3,4-b]pyridinyl, 7H-purinyl, indolizinyl, imidazo[1,2-a]pyridinyl, imidazo[1,5-a]pyridinyl, pyrazolo[1,5-a]pyridinyl, pyrrolo[1,2-b]pyridazinyl, imidazo[1,2-c]pyrimidinyl, quinolyl, isoquinolyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-d]pyrimidinyl, 1,2,3,4-tetrahydropyrido[2,3-b]pyrazinyl, 2,3-dihydrobenzo[b][1,4]dioxinyl, 3,4-dihydro-2H-pyrido[3,2-b][1,4]oxazinyl, 2,3-dihydro-1H-benzo[d]imidazolyl, benzo[d]thiazolyl, 2,3-dihydro-1H-pyrrolo[2,3-b]pyridinyl, [1,2,4]triazolo[1,5-a]pyridinyl, 2,3-dihydro-1H-imidazo[4,5-b]pyridinyl, tetrazolo[1,5-a]pyridinyl, 7H-pyrrolo[2,3-d]pyrimidinyl, pyrazolo[1,5-a]pyrimidinyl, imidazo[1,2-a]pyrimidinyl, 4,5-dihydro-1H-pyrazolo[3,4-d]pyrimidinyl, 2,3,6,7-tetrahydro-1H-purinyl, 5H-pyrrolo[2,3-b]pyrazinyl, imidazo[1,2-a]pyrazinyl, imidazo[1,2-b]pyridazinyl, and 4,5,6,7-tetrahydropyrazolo[1,5-a]pyrazinyl.

“Heteroarylene” refers to heteroaryl groups which are attached through two ring atoms of the group, where heteroaryl is defined above. They generally have a specified number of carbon atoms in their ring or rings (e.g., C₃₅ heteroarylene refers to a heteroarylene group having 3 to 5 carbon atoms and, e.g., 1 to 4 (i.e., 1, 2, 3 or 4) heteroatoms, as ring members). Examples of heteroarylene groups include the multivalent analogs of the heteroaryl groups described above, such as pyridine-2,3-diyl, pyridine-3,4-diyl, pyrazole-4,5-diyl, pyrazole-3,4-diyl, and the like.

“Non-aromatic heterocyclic/heterocyclyl” (including “3- to 8-membered non-aromatic heterocyclic group)” refers to heterocyclic group other than heteroaryl groups as mentioned above. Preferable examples of the “non-aromatic heterocyclic group” include 3- to 8-membered monocyclic non-aromatic heterocyclic groups such as aziridinyl, oxiranyl, thiiranyl, azetidinyl, oxetanyl, thietanyl, tetrahydrothienyl, tetrahydrofuranyl, pyrrolinyl, pyrrolidinyl, imidazolinyl, imidazolidinyl, oxazolinyl, oxazolidinyl, pyrazolinyl, pyrazolidinyl, thiazolinyl, thiazolidinyl, tetrahydroisothiazolyl, tetrahydrooxazolyl, tetrahydroisooxazolyl, piperidinyl, piperazinyl, tetrahydropyridinyl, dihydropyridinyl, dihydrothiopyranyl, tetrahydropyrimidinyl, tetrahydropyridazinyl, dihydropyranyl, tetrahydropyranyl, tetrahydrothiopyranyl, morpholinyl, thiomorpholinyl, azepanyl, diazepanyl, azepinyl, oxepanyl, azocanyl, diazocanyl and the like; and 9- to 14-membered fused polycyclic (preferably bi or tricyclic) non-aromatic heterocyclic groups such as dihydrobenzofuranyl, dihydrobenzimidazolyl, dihydrobenzoxazolyl, dihydrobenzothiazolyl, dihydrobenzisothiazolyl, dihydronaphtho[2,3-b]thienyl, tetrahydroisoquinolyl, tetrahydroquinolyl, 4H-quinolizinyl, indolinyl, isoindolinyl, tetrahydrothieno[2,3-c]pyridinyl, tetrahydrobenzazepinyl, tetrahydroquinoxalinyl, tetrahydrophenanthridinyl, hexahydrophenothiazinyl, hexahydrophenoxazinyl, tetrahydrophthalazinyl, tetrahydronaphthyridinyl, tetrahydroquinazolinyl, tetrahydrocinnolinyl, tetrahydrocarbazolyl, tetrahydro-β-carbolinyl, tetrahydroacrydinyl, tetrahydrophenazinyl, tetrahydrothioxanthenyl, octahydroisoquinolyl and the like.

In the present specification, examples of the “nitrogen-containing heterocyclic group” include a “heterocyclic group” containing at least one nitrogen atom as a ring-constituting atom.

In the present specification, examples of the “optionally substituted heterocyclic group” include a heterocyclic group optionally having substituent(s) selected from the substituent group A as described later.

“Oxo” refers to a double bonded oxygen (═O).

Examples of the “substituent” (including “hetero-containing substituents”) include a halogen atom, a cyano group, a nitro group, an optionally substituted hydrocarbon group, an optionally substituted heterocyclic group, an acyl group, an optionally substituted amino group, an optionally substituted carbamoyl group, an optionally substituted thiocarbamoyl group, an optionally substituted sulfamoyl group, an optionally substituted hydroxy group, an optionally substituted sulfanyl (SH) group and an optionally substituted silyl group. Examples of the “hydrocarbon group” (including “hydrocarbon group” of “optionally substituted hydrocarbon group”) include a C₁₋₆ alkyl group, a C₂₋₆ alkenyl group, a C₂₋₆ alkynyl group, a C₃₋₁₀ cycloalkyl group, a C₃₋₁₀ cycloalkenyl group, a C₆₋₁₄ aryl group and a C₇₋₁₆ aralkyl group.

“Hetero-containing substituents” refers to substituents containing at least one heteroatom. Examples of the “hetero-containing substituents” include a halogen atom, a cyano group, a nitro group, a heterocyclyl group, a heteroaryl group, an alkyl group substituted by hetero-containing substituents (such as halo-alkyl, amino-alkyl, cyano-alkyl, alkoxy-alkyl, and the like), a cycloalkyl substituted by hetero-containing substituents (such as halo-cycloalkyl, cyano-cycloalkyl, hydroxy-cycloalkyl, and the like), an optionally substituted alkoxy group, and the like.

Examples of the “optionally substituted hydrocarbon group” include a hydrocarbon group optionally having substituent(s) selected from the following substituent group A.

### [Substituent Group A]

(1) a halogen atom,
(2) a nitro group,
(3) a cyano group,
(4) an oxo group,
(5) a hydroxy group,
(6) an optionally halogenated C₁₋₆ alkoxy group,
(7) a C₆₋₁₄ aryloxy group (e.g., phenoxy, naphthoxy),
(8) a C₇₋₁₆ aralkyloxy group (e.g., benzyloxy),
(9) a 5- to 14-membered aromatic heterocyclyloxy group (e.g., pyridyloxy),
(10) a 3- to 14-membered non-aromatic heterocyclyloxy group (e.g., morpholinyloxy, piperidinyloxy),
(11) a C₁₋₆ alkyl-carbonyloxy group (e.g., acetoxy, propanoyloxy),
(12) a C₆₋₁₄ aryl-carbonyloxy group (e.g., benzoyloxy, 1-naphthoyloxy, 2-naphthoyloxy),
(13) a C₁₋₆ alkoxy-carbonyloxy group (e.g., methoxycarbonyloxy, ethoxycarbonyloxy, propoxycarbonyloxy, butoxycarbonyloxy),
(14) a mono- or di-C₁₋₆ alkyl-carbamoyloxy group (e.g., methylcarbamoyloxy, ethylcarbamoyloxy, dimethylcarbamoyloxy, diethylcarbamoyloxy),
(15) a C₆₋₁₄ aryl-carbamoyloxy group (e.g., phenylcarbamoyloxy, naphthylcarbamoyloxy),
(16) a 5- to 14-membered aromatic heterocyclylcarbonyloxy group (e.g., nicotinoyloxy),
(17) a 3- to 14-membered non-aromatic heterocyclylcarbonyloxy group (e.g., morpholinylcarbonyloxy, piperidinylcarbonyloxy),
(18) an optionally halogenated C₁₋₆ alkylsulfonyloxy group (e.g., methylsulfonyloxy, trifluoromethylsulfonyloxy),
(19) a C₆₋₁₄ arylsulfonyloxy group optionally substituted by a C₁₋₆ alkyl group (e.g., phenylsulfonyloxy, toluenesulfonyloxy),
(20) an optionally halogenated C₁₋₆ alkylthio group,
(21) a 5- to 14-membered aromatic heterocyclic group,
(22) a 3- to 14-membered non-aromatic heterocyclic group,
(23) a formyl group,
(24) a carboxy group,
(25) an optionally halogenated C₁₋₆ alkyl-carbonyl group,
(26) a C₆₋₁₄ aryl-carbonyl group,
(27) a 5- to 14-membered aromatic heterocyclylcarbonyl group,
(28) a 3- to 14-membered non-aromatic heterocyclylcarbonyl group,
(29) a C₁₋₆ alkoxy-carbonyl group,
(30) a C₆₋₁₄ aryloxy-carbonyl group (e.g., phenyloxycarbonyl, 1-naphthyloxycarbonyl, 2-naphthyloxycarbonyl),
(31) a C₇₋₁₆ aralkyloxy-carbonyl group (e.g., benzyloxycarbonyl, phenethyloxycarbonyl),
(32) a carbamoyl group,
(33) a thiocarbamoyl group,
(34) a mono- or di-C₁₋₆ alkyl-carbamoyl group,
(35) a C₆₋₁₄ aryl-carbamoyl group (e.g., phenylcarbamoyl),
(36) a 5- to 14-membered aromatic heterocyclylcarbamoyl group (e.g., pyridylcarbamoyl, thienylcarbamoyl),
(37) a 3- to 14-membered non-aromatic heterocyclylcarbamoyl group (e.g., morpholinylcarbamoyl, piperidinylcarbamoyl),
(38) an optionally halogenated C₁₋₆ alkylsulfonyl group,
(39) a C₆₋₁₄ arylsulfonyl group,
(40) a 5- to 14-membered aromatic heterocyclylsulfonyl group (e.g., pyridylsulfonyl, thienylsulfonyl),
(41) an optionally halogenated C₁₋₆ alkylsulfinyl group,
(42) a C₆₋₁₄ arylsulfinyl group (e.g., phenylsulfinyl, 1-naphthylsulfinyl, 2-naphthylsulfinyl),
(43) a 5- to 14-membered aromatic heterocyclylsulfinyl group (e.g., pyridylsulfinyl, thienylsulfinyl),
(44) an amino group,
(45) a mono- or di-C₁₋₆ alkylamino group (e.g., methylamino, ethylamino, propylamino, isopropylamino, butylamino, dimethylamino, diethylamino, dipropylamino, dibutylamino, N-ethyl-N-methylamino),
(46) a mono- or di-C₆₋₁₄ arylamino group (e.g., phenylamino),
(47) a 5- to 14-membered aromatic heterocyclylamino group (e.g., pyridylamino),
(48) a C₇₋₁₆ aralkylamino group (e.g., benzylamino),
(49) a formylamino group,
(50) a C₁₋₆ alkyl-carbonylamino group (e.g., acetylamino, propanoylamino, butanoylamino),
(51) a (C₁₋₆ alkyl)(C₁₋₆ alkyl-carbonyl)amino group (e.g., N-acetyl-N-methylamino),
(52) a C₆₋₁₄ aryl-carbonylamino group (e.g., phenylcarbonylamino, naphthylcarbonylamino),
(53) a C₁₋₆ alkoxy-carbonylamino group (e.g., methoxycarbonylamino, ethoxycarbonylamino, propoxycarbonylamino, butoxycarbonylamino, tert-butoxycarbonylamino),
(54) a C₇₋₁₆ aralkyloxy-carbonylamino group (e.g., benzyloxycarbonylamino),
(55) a C₁₋₆ alkylsulfonylamino group (e.g., methylsulfonylamino, ethylsulfonylamino),
(56) a C₆₋₁₄ arylsulfonylamino group optionally substituted by a C₁₋₆ alkyl group (e.g., phenylsulfonylamino, toluenesulfonylamino),
(57) an optionally halogenated C₁₋₆ alkyl group,
(58) a C₂₋₆ alkenyl group,
(59) a C₂₋₆ alkynyl group,
(60) a C₃₋₁₀ cycloalkyl group,
(61) a C₃₋₁₀ cycloalkenyl group and
(62) a C₆₋₁₄ aryl group.

The number of the above-mentioned substituents in the “optionally substituted hydrocarbon group” is, for example, 1 to 5, preferably 1 to 3. When the number of the substituents is two or more, the respective substituents may be the same or different.

“Leaving group” refers to any group that leaves a molecule during a fragmentation process, including substitution reactions, elimination reactions, and addition-elimination reactions. Leaving groups may be nucleofugal, in which the group leaves with a pair of electrons that formerly served as the bond between the leaving group and the molecule, or may be electrofugal, in which the group leaves without the pair of electrons. The ability of a nucleofugal leaving group to leave depends on its base strength, with the strongest bases being the poorest leaving groups. Common nucleofugal leaving groups include nitrogen (e.g., from diazonium salts); sulfonates, including alkylsulfonates (e.g., mesylate), fluoroalkylsulfonates (e.g., triflate, hexaflate, nonaflate, and tresylate), and arylsulfonates (e.g., tosylate, brosylate, closylate, and nosylate). Others include carbonates, halide ions, carboxylate anions, phenolate ions, and alkoxides. Some stronger bases, such as NH₂⁻ and OH can be made better leaving groups by treatment with an acid. Common electrofugal leaving groups include the proton, CO₂, and metals.

“Opposite enantiomer” refers to a molecule that is a non-superimposable mirror image of a reference molecule, which may be obtained by inverting all the stereogenic centers of the reference molecule. For example, if the reference molecule has S absolute stereochemical configuration, then the opposite enantiomer has R absolute stereochemical configuration. Likewise, if the reference molecule has S,S absolute stereochemical configuration, then the opposite enantiomer has R,R stereochemical configuration, and so on.

“Stereoisomer” and “stereoisomers” of a compound with given stereochemical configuration refer to the opposite enantiomer of the compound and to any diastereoisomers, including geometrical isomers (Z/E) of the compound. For example, if a compound has S,R,Z stereochemical configuration, its stereoisomers would include its opposite enantiomer having R,S,Z configuration, and its diastereomers having S,S,Z configuration, R,R,Z configuration, S,R,E configuration, R,S,E configuration, S,S,E configuration, and R,R,E configuration. If the stereochemical configuration of a compound is not specified, then “stereoisomer” refers to any one of the possible stereochemical configurations of the compound.

“Substantially pure stereoisomer” and variants thereof refer to a sample containing a compound having a specific stereochemical configuration and which comprises at least about 95% of the sample.

“Pure stereoisomer” and variants thereof refer to a sample containing a compound having a specific stereochemical configuration and which comprises at least about 99.5% of the sample.

“Subject” refers to a mammal, including a human.

“Pharmaceutically acceptable” substances refer to those substances which are suitable for administration to subjects.

“Treating” refers to reversing, alleviating, inhibiting the progress of, or preventing a disease, disorder or condition to which such term applies, or to reversing, alleviating, inhibiting the progress of, or preventing one or more symptoms of such disease, disorder or condition.

“Treatment” refers to the act of “treating,” as defined immediately above.

“Drug,” “drug substance,” “active pharmaceutical ingredient,” and the like, refer to a compound (e.g., compounds of Formula (I), including subgeneric compounds and compounds specifically named in the specification, or pharmaceutically acceptable salts thereof, or solvates thereof, or hydrates thereof) that may be used for treating a subject in need of treatment.

“Effective amount” of a drug, “therapeutically effective amount” of a drug, and the like, refer to the quantity of the drug that may be used for treating a subject and may depend on the weight and age of the subject and the route of administration, among other things.

“Excipient” refers to any diluent or vehicle for a drug.

“Medicament” refers to the combination of one or more drug substances and one or more excipients. Sometimes such combination is also described as “formulation” or “pharmaceutical composition”.

“Drug product,” “pharmaceutical dosage form,” “dosage form,” “final dosage form” and the like, refer to a pharmaceutical composition or a medicament suitable for treating a subject in need of treatment and generally may be in the form of tablets, capsules, sachets containing powder or granules, liquid solutions or suspensions, patches, films, and the like.

“Disease, disorder or condition associated with NLRP3” and similar phrases relate to a disease, disorder or condition in a subject for which inhibition of the NLRP3 inflammasome pathway may provide a therapeutic or prophylactic benefit.

The following abbreviations may be used in the specification: Ac (acetyl); Ac₂O (acetic anhydride); ACN (acetonitrile); AIBN (azo-bis-isobutyronitrile); AmPhos (bis(di-tert-butyl(4-dimethylaminophenyl)phosphine)dichloropalladium(II)); API (active pharmaceutical ingredient); aq (aqueous); BINAP (2,2′-bis(diphenylphosphino)-1,1′-binaphthyl); Bn (benzyl); Boc (tert-butoxycarbonyl); BrettPhos (2-(dicyclohexylphosphino)3,6-dimethoxy-2′,4′,6′-triisopropyl-1,1′-biphenyl); BrettPhos-Pd-G3 ([(2-di-cyclohexylphosphino-3,6-dimethoxy-2′,4′,6′-triisopropyl-1,1′-biphenyl)-2-(2′-amino-1,1′-biphenyl)]palladium(II)methanesulfonate); Cbz (carbobenzyloxy); Troc (2,2,2-trichloroethoxycarbonyl); dba (dibenzylideneacetone); DBU (1,8-diazabicyclo[5.4.0]undec-7-ene); DCC (1,3-dicyclohexylcarbodiimide); DCE (1,1-dichloroethane); DCM (dichloromethane); DEA (diethylamine); DIAD (diisopropyl azodicarboxylate); DIPEA (N,N-diisopropylethylamine, Hunig's Base); DMA (N,N-dimethylacetamide); DMAP (4-dimethylaminopyridine); DME (1,2-dimethoxyethane); DMF (N,N-dimethylformamide); DMP (Dess-Martin periodinane); DMSO (dimethylsulfoxide); dppf (1,1′-bis(diphenylphosphino)ferrocene); DTT (dithiothreitol); EC₅₀ (effective concentration at half maximal response); EDA (ethoxylated dodecyl alcohol, Brj®35); EDC (N-(3-dimethylaminopropyl)-N′-ethylcarbodiimide); EDTA (ethylenediaminetetraacetic acid); ee (enantiomeric excess); ELS (evaporative light scattering); eq (equivalents); Et (ethyl); Et₃N (triethylamine); EtOAc (ethyl acetate); EtOH (ethanol); FA (formic acid); HATU (2-(3H-[1,2,3]triazolo[4,5-b]pyridin-3-yl)-1,1,3,3-tetramethyluronium hexafluorophosphate(V)); HEPES (4-(2-hydroxyethyl)piperazine-1-ethanesulfonic acid); HOAc (acetic acid); HOBt (1H-benzo[d][1,2,3]triazol-1-ol); IC₅₀ (concentration at 50% inhibition); IPA (isopropanol); IPAc (isopropyl acetate); IPE (isopropyl ether); LDA (lithium diisopropylamide); LiHMDS (lithium bis(trimethylsilyl)amide); mCPBA (m-chloroperoxybenzoic acid); Me (methyl); MeOH (methanol); MTBE (methyl tert-butyl ether); mp (melting point); NaOt-Bu (sodium tertiary butoxide); NMM (N-methylmorpholine); NMP 1-methylpyrrolidin-2-one); OTf (triflate); PE (petroleum ether); Ph (phenyl); pEC₅₀ (−log₁₀(EC₅₀), where EC₅₀ is given in molar (M) units); pIC₅₀ (−log₁₀(IC₅₀), where IC₅₀ is given in molar (M) units); PMB (p-methoxylbenzyl); Pr (propyl); c-Pr (cyclopropyl), i-Pr (isopropyl); PTFE (polytetrafluoroethylene); PyBOP ((benzotriazol-1-yloxy)tripyrrolidinophosphonium hexafluorophosphate); PyBroP® (bromotripyrrolidinophosphonium hexafluorophosphate); PCy₃ (tricyclohexylphosphine); RT (room temperature, approximately 20 °C to 25 °C); SFC (supercritical fluid chromatography); T3P (2,4,6-tripropyl-1,3,5,2,4,6-trioxatriphosphinane 2,4,6-trioxide); TCEP (tris(2-carboxyethyl)phosphine); TFA (trifluoroacetic acid); TFAA (2,2,2-trifluoroacetic anhydride); THE (tetrahydrofuran); TMS (trimethylsilyl); Tris buffer (2-amino-2-hydroxymethyl-propane-1,3-diol buffer); XPhos (2-dicyclohexylphosphino-2′,4′,6′-triisopropylbiphenyl); and XPhos-Pd-G2 (chloro(2-dicyclohexylphosphino-2′,4′,6′-triisopropyl-1,1′-biphenyl)[2-(2′-amino-1,1′-biphenyl)]palladium(II)).

As described below, this disclosure concerns compounds of Formula (I), or pharmaceutically acceptable salts thereof, or solvates thereof, or hydrates thereof (collectively, sometimes to be referred to as compound (I) in the present specification). This disclosure also concerns materials and methods for preparing compounds of Formula (I), or pharmaceutically acceptable salts thereof, or solvates thereof, or hydrates thereof, medicaments which contain them, and the use of compounds of Formula (I), or pharmaceutically acceptable salts thereof, or solvates thereof, or hydrates thereof, (optionally in combination with other pharmacologically active agent(s)) for treating neurodegenerative diseases such as Parkinson's disease, Alzheimer's disease, Huntington's disease, amyotrophic lateral sclerosis, prion disease and other diseases, disorders and/or conditions associated with NLRP3.

The definition of each variable in Formula (I) is explained in detail in the following.

X is S or NR⁷ wherein R⁷ is as defined below.

In one embodiment, X is S.

In one embodiment, X is NR⁷ wherein R⁷ is as defined below.

R⁷ is a hydrogen atom, or an optionally substituted C₁₋₆ alkyl group.

R⁷ is preferably an optionally substituted C₁₋₆ alkyl group.

R⁷ is more preferably a C₁₋₆ alkyl group (e.g., methyl).

X is preferably S or NR⁷ wherein R⁷ is a C₁₋₆ alkyl group (e.g., methyl).

X is particularly preferably S.

Y is CR⁸ wherein R⁸ is as defined below, or N.

In one embodiment, Y is CR⁸ wherein R⁸ is as defined below.

In one embodiment, Y is N.

R⁸ is a hydrogen atom, a halogen atom, an optionally substituted C₁₋₆ alkyl group, an optionally substituted C₃₋₈ cycloalkyl, or an optionally substituted C₁₋₆ alkoxy group.

R⁸ is preferably a halogen atom, an optionally substituted C₁₋₆ alkyl group, an optionally substituted C₃₋₈ cycloalkyl, or an optionally substituted C₁₋₆ alkoxy group.

R⁸ is more preferably a halogen atom, an optionally substituted C₁₋₆ alkyl group, or an optionally substituted C₃₋₈ cycloalkyl.

R⁸ is further more preferably an optionally substituted C₁₋₆ alkyl group, or an optionally substituted C₃₋₈ cycloalkyl.

R⁸ is still more preferably an optionally substituted C₁₋₆ alkyl group.

R⁸ is even more preferably an optionally halogenated C₁₋₆ alkyl group.

R⁸ is particularly preferably a C₁₋₆ alkyl group.

As another embodiment, R⁸ is preferably
(1) a hydrogen atom,
(2) a halogen atom (e.g., a chlorine atom),
(3) a C₁₋₆ alkyl group (e.g., methyl, ethyl, isopropyl) optionally substituted by 1 to 3 halogen atoms (e.g., a fluorine atom),
(4) a C₃₋₈ cycloalkyl group (e.g., cyclopropyl) optionally substituted by 1 to 3 halogen atoms (e.g., a fluorine atom), or
(5) a C₁₋₆ alkoxy group (e.g., methoxy) optionally substituted by 1 to 3 halogen atoms (e.g., a fluorine atom).

R⁸ is more preferably
(1) a halogen atom (e.g., a chlorine atom),
(2) a C₁₋₆ alkyl group (e.g., methyl, ethyl, isopropyl) optionally substituted by 1 to 3 halogen atoms (e.g., a fluorine atom),
(3) a C₃₋₈ cycloalkyl group (e.g., cyclopropyl) optionally substituted by 1 to 3 halogen atoms (e.g., a fluorine atom), or
(4) a C₁₋₆ alkoxy group (e.g., methoxy) optionally substituted by 1 to 3 halogen atoms (e.g., a fluorine atom).

R⁸ is further more preferably
(1) a halogen atom (e.g., a chlorine atom),
(2) a C₁₋₆ alkyl group (e.g., methyl, ethyl, isopropyl) optionally substituted by 1 to 3 halogen atoms (e.g., a fluorine atom), or
(3) a C₃₋₈ cycloalkyl group (e.g., cyclopropyl) optionally substituted by 1 to 3 halogen atoms (e.g., a fluorine atom).

R⁸ is still more preferably
(1) a C₁₋₆ alkyl group (e.g., methyl, ethyl, isopropyl) optionally substituted by 1 to 3 halogen atoms (e.g., a fluorine atom), or
(2) a C₃₋₈ cycloalkyl group (e.g., cyclopropyl) optionally substituted by 1 to 3 halogen atoms (e.g., a fluorine atom).

R⁸ is even more preferably a C₁₋₆ alkyl group (e.g., methyl, ethyl, isopropyl) optionally substituted by 1 to 3 halogen atoms (e.g., a fluorine atom).

R⁸ is particularly preferably a C₁₋₆ alkyl group (e.g., methyl, ethyl).

Y is preferably CR⁸ wherein R⁸ is
(1) a hydrogen atom,
(2) a halogen atom (e.g., a chlorine atom),
(3) a C₁₋₆ alkyl group (e.g., methyl, ethyl, isopropyl) optionally substituted by 1 to 3 substituents selected from (i) halogen atoms (e.g., a fluorine atom) and (ii) a C₁₋₆ alkoxy group (e.g., methoxy) optionally substituted by 1 to 3 halogen atoms (e.g., fluorine atom),
(4) a C₃₋₈ cycloalkyl group (e.g., cyclopropyl) optionally substituted by 1 to 3 halogen atoms (e.g., a fluorine atom), or
(5) a C₁₋₆ alkoxy group (e.g., methoxy) optionally substituted by 1 to 3 halogen atoms (e.g., a fluorine atom), or N.

Y is more preferably CR⁸ wherein R⁸ is
(1) a hydrogen atom,
(2) a halogen atom (e.g., a chlorine atom),
(3) a C₁₋₆ alkyl group (e.g., methyl, ethyl, isopropyl) optionally substituted by 1 to 3 substituents selected from (i) halogen atoms (e.g., a fluorine atom) and (ii) a C₁₋₆ alkoxy group (e.g., methoxy) optionally substituted by 1 to 3 halogen atoms (e.g., fluorine atom),
(4) a C₃₋₈ cycloalkyl group (e.g., cyclopropyl) optionally substituted by 1 to 3 halogen atoms (e.g., a fluorine atom), or
(5) a C₁₋₆ alkoxy group (e.g., methoxy) optionally substituted by 1 to 3 halogen atoms (e.g., a fluorine atom).

Y is further more preferably CR⁸ wherein R⁸ is
(1) a halogen atom (e.g., a chlorine atom),
(2) a C₁₋₆ alkyl group (e.g., methyl, ethyl, isopropyl) optionally substituted by 1 to 3 substituents selected from (i) halogen atoms (e.g., a fluorine atom) and (ii) a C₁₋₆ alkoxy group (e.g., methoxy) optionally substituted by 1 to 3 halogen atoms (e.g., fluorine atom),
(3) a C₃₋₈ cycloalkyl group (e.g., cyclopropyl) optionally substituted by 1 to 3 halogen atoms (e.g., a fluorine atom), or
(4) a C₁₋₆ alkoxy group (e.g., methoxy) optionally substituted by 1 to 3 halogen atoms (e.g., a fluorine atom).

Y is even more preferably CR⁸ wherein R⁸ is a C₁₋₆ alkyl group (e.g., methyl, ethyl, isopropyl) optionally substituted by 1 to 3 substituents selected from (i) halogen atoms (e.g., a fluorine atom) and (ii) a C₁₋₆ alkoxy group (e.g., methoxy) optionally substituted by 1 to 3 halogen atoms (e.g., fluorine atom).

Y is particularly preferably CR⁸ wherein R⁸ is a C₁₋₆ alkyl group (e.g., methyl, ethyl).

R¹ is an optionally substituted 4-membered heterocyclic group or 5- or 6-membered heterocyclic group, an optionally substituted C₁₋₆ alkyl group, or an optionally substituted C₃₋₁₀ cycloalkyl group.

R¹ is preferably
(1) an optionally substituted 4-membered heterocyclic group or 5- or 6-membered heterocyclic group,
(2) an C₁₋₆ alkyl group substituted by hetero-containing substituent(s), or
(3) an C₃₋₁₀ cycloalkyl group substituted by hetero-containing substituent(s).

R¹ is more preferably an optionally substituted 4-membered heterocyclic group or 5- or 6-membered heterocyclic group.

R¹ is further more preferably an optionally substituted 4-membered heterocyclic group or 5- or 6-membered non-aromatic heterocyclic group.

R¹ is still more preferably an optionally substituted 4-membered heterocyclic group or 5- or 6-membered nitrogen-containing non-aromatic heterocyclic group.

R¹ is even more preferably a group of Formula (A′):

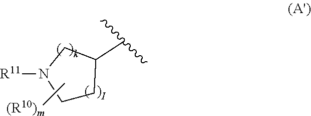

wherein
R¹¹ is a hydrogen atom, or a substituent;
R¹⁰ are each independently a substituent, or two R¹⁰ are taken together to form an oxo group;
m is 0, 1, 2, 3 or 4;
k is 0, 1 or 2;
l is 0, 1 or 2; and
k+l is 1 or 2.

R¹ is still even more preferably a group of Formula (A):

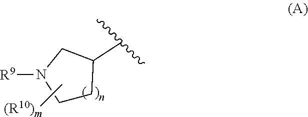

wherein
R⁹ is a hydrogen atom, or a substituent;
R¹⁰ are each independently a substituent, or two R¹⁰ are taken together to form an oxo group;
m is 0, 1, 2, 3 or 4; and
n is 1 or 2.

R⁹ is preferably
(1) a hydrogen atom,
(2) an optionally substituted C₁₋₆ alkyl group,
(3) an optionally substituted C₃₋₈ cycloalkyl group,
(4) an optionally substituted C₁₋₆ alkyl-carbonyl group,
(5) an optionally substituted C₃₋₈ cycloalkyl-carbonyl group,
(6) an optionally substituted C₁₋₆ alkoxy-carbonyl group,
(7) an optionally substituted C₇₋₁₆ aralkyloxy-carbonyl group,
(8) an optionally substituted mono- or di-C₁₋₆ alkyl-carbamoyl group,
(9) an optionally substituted C₁₋₆ alkylsulfonyl group,
(10) an optionally substituted C₃₋₈ cycloalkylsulfonyl group, or
(11) an optionally substituted 3- to 8-membered non-aromatic heterocyclic group.

R¹⁰ is preferably
(1) a halogen atom,
(2) an optionally substituted C₁₋₆ alkyl group, or
(3) an optionally substituted C₁₋₆ alkoxy group, or
two R¹⁰ are taken together to form an oxo group.

m is preferably 0, 1 or 2.

As another embodiment, R¹ is preferably
(1) a 4-membered heterocyclic group or 5- or 6-membered heterocyclic group (preferably a 4-membered heterocyclic group or 5- or 6-membered non-aromatic heterocyclic group (e.g., oxetanyl, pyrrolidinyl, piperidyl, tetrahydrofuranyl, tetrahydropyranyl), wherein the heterocyclic groups are optionally substituted by 1 to 3 substituents selected from
   (a) a halogen atom (e.g., a fluorine atom),
   (b) an oxo group,
   (c) a C₁₋₆ alkyl group (e.g., methyl, ethyl) optionally substituted by 1 to 3 substituents selected from
      (i) a halogen atom (e.g., a fluorine atom), and
      (ii) a C₁₋₆ alkoxy group (e.g., methoxy),
   (d) a C₃₋₈ cycloalkyl group (e.g., cyclopropyl),
   (e) a hydroxy group,
   (f) a C₁₋₆ alkoxy group (e.g., methoxy),
   (g) a C₁₋₆ alkyl-carbonyl group (e.g., acetyl, propanoyl, 2-methylpropanoyl) optionally substituted by 1 to 3 substituents selected from
      (i) a halogen atom (e.g., a fluorine atom),
      (ii) a hydroxy group,
      (iii) a C₁₋₆ alkoxy group (e.g., methoxy), and
      (iv) a C₃₋₈ cycloalkyl group (e.g., cyclopropyl),
   (h) a C₃₋₈ cycloalkyl-carbonyl group (e.g., cyclopropylcarbonyl) optionally substituted by 1 to 3 substituents selected from
      (i) a halogen atom (e.g., a fluorine atom),
      (ii) a cyano group, and
      (iii) a hydroxy group,
   (i) a C₁₋₆ alkoxy-carbonyl group (e.g., methoxycarbonyl, ethoxycarbonyl) optionally substituted by 1 to 3 halogen atoms (e.g., a chlorine atom),
   (j) a C₃₋₈ cycloalkoxy-carbonyl group (e.g. cyclopropoxycarbonyl),
   (k) a C₇₋₁₆ aralkyloxy-carbonyl group (e.g., benzyloxycarbonyl),
   (l) a mono- or di-C₁₋₆ alkyl-carbamoyl group (e.g., methylcarbamoyl),
   (m) a C₁₋₆ alkylsulfonyl group (e.g., methylsulfonyl),
   (n) a C₃₋₈ cycloalkylsulfonyl group (e.g., cyclopropylsulfonyl), and
   (o) a 3- to 8-membered non-aromatic heterocyclic group (e.g., tetrahydrofuranyl),
(2) a C₁₋₆ alkyl group (e.g., methyl, ethyl, propyl, isopropyl, butyl, isobutyl, sec-butyl, isopentyl) optionally substituted by 1 to 3 substituents selected from
   (a) a hydroxy group,
   (b) a C₁₋₆ alkoxy group (e.g., methoxy) optionally substituted by halogen atoms (e.g., fluorine atom),
   (c) a C₃₋₈ cycloalkyl group (e.g., cyclopropyl, cyclobutyl) optionally substituted by 1 to 3 halogen atoms (e.g., a fluorine atom),
   (d) a mono- or di-C₁₋₆ alkylamino group (e.g., dimethylamino),
   (e) a C₁₋₆ alkyl-carbonylamino group (e.g., acetylamino),
   (f) a 3- to 8-membered non-aromatic heterocyclic group (e.g., oxetanyl, morpholinyl, tetrahydrofuranyl) optionally substituted by a hydroxy group, and
   (g) a halogen atom (e.g., fluorine atom), and
   (h) a 5- to 14-membered aromatic heterocyclyl group (e.g., pyridyl), or
(3) a C₃₋₁₀ cycloalkyl group (the C₃₋₁₀ cycloalkyl group may be a spiro ring group or a bridged spiro ring group, e.g., cyclopropyl, cyclobutyl, cyclopentyl, cyclohexyl, spiro[3.3]heptanyl, bicyclo[1.1.1]pentyl, bicyclo[2.2.1]heptanyl, adamantyl) optionally substituted by 1 to 3 substituents selected from
   (a) a hydroxy group,
   (b) a C₁₋₆ alkyl group (e.g., methyl) optionally substituted by 1 to 3 halogen atoms (e.g., fluorine atom), and
   (c) a C₁₋₆ alkoxy group (e.g., methoxy) optionally substituted by 1 to 3 halogen atoms (e.g. fluorine atom).

R¹ is more preferably
(1) a 4-membered heterocyclic group or 5- or 6-membered heterocyclic group (preferably a 4- to 6-membered non-aromatic heterocyclic group (e.g., oxetanyl, pyrrolidinyl, piperidyl, tetrahydrofuranyl, tetrahydropyranyl)), wherein the heterocyclic groups are optionally substituted by 1 to 3 substituents selected from
   (a) a halogen atom (e.g., a fluorine atom),
   (b) an oxo group,
   (c) a C₁₋₆ alkyl group (e.g., methyl, ethyl) optionally substituted by 1 to 3 substituents selected from
      (i) a halogen atom (e.g., a fluorine atom), and
      (ii) a C₁₋₆ alkoxy group (e.g., methoxy),
   (d) a C₃₋₈ cycloalkyl group (e.g., cyclopropyl),
   (e) a hydroxy group,
   (f) a C₁₋₆ alkoxy group (e.g., methoxy),
   (g) a C₁₋₆ alkyl-carbonyl group (e.g., acetyl, propanoyl, 2-methylpropanoyl) optionally substituted by 1 to 3 substituents selected from
      (i) a halogen atom (e.g., a fluorine atom),
      (ii) a hydroxy group,
      (iii) a C₁₋₆ alkoxy group (e.g., methoxy), and
      (iv) a C₃₋₈ cycloalkyl group (e.g., cyclopropyl),
   (h) a C₃₋₈ cycloalkyl-carbonyl group (e.g., cyclopropylcarbonyl) optionally substituted by 1 to 3 substituents selected from
      (i) a halogen atom (e.g., a fluorine atom),
      (ii) a cyano group, and
      (iii) a hydroxy group,
   (i) a C₁₋₆ alkoxy-carbonyl group (e.g., methoxycarbonyl, ethoxycarbonyl) optionally substituted by 1 to 3 halogen atoms (e.g., a chlorine atom),
   (j) a C₃₋₈ cycloalkoxy-carbonyl group (e.g. cyclopropoxycarbonyl)
   (k) a C₇₋₁₆ aralkyloxy-carbonyl group (e.g., benzyloxycarbonyl),
   (l) a mono- or di-C₁₋₆ alkyl-carbamoyl group (e.g., methylcarbamoyl),
   (m) a C₁₋₆ alkylsulfonyl group (e.g., methylsulfonyl),
   (n) a C₃₋₈ cycloalkylsulfonyl group (e.g., cyclopropylsulfonyl), and
   (o) a 3- to 8-membered non-aromatic heterocyclic group (e.g., tetrahydrofuranyl),
(2) a C₁₋₆ alkyl group (e.g., methyl, ethyl, propyl, isopropyl, butyl, isobutyl, sec-butyl, isopentyl) substituted by 1 to 3 substituents selected from
   (a) a hydroxy group,
   (b) a C₁₋₆ alkoxy group (e.g., methoxy) optionally substituted by 1 to 3 halogen atoms (e.g., fluorine atom),
   (c) a C₃₋₈ cycloalkyl group (e.g., cyclopropyl, cyclobutyl) substituted by 1 to 3 halogen atoms (e.g., a fluorine atom),
   (d) a mono- or di-C₁₋₆ alkylamino group (e.g., dimethylamino),
   (e) a C₁₋₆ alkyl-carbonylamino group (e.g., acetylamino),
   (f) a 3- to 8-membered non-aromatic heterocyclic group (e.g., oxetanyl, morpholinyl, tetrahydrofuranyl) optionally substituted by a hydroxy group,
   (g) a halogen atom (e.g., fluorine atom), and
   (h) a 5- to 14-membered aromatic heterocyclyl group (e.g., pyridyl), or
(3) a C₃₋₁₀ cycloalkyl group (the C₃₋₁₀ cycloalkyl group may be a spiro ring group or a bridged spiro ring group, e.g., cyclobutyl, cyclopentyl, cyclohexyl, spiro[3.3]heptanyl, bicyclo[1.1.1]pentyl, bicyclo[2.2.1]heptanyl, adamantyl) substituted by one hydroxy group, and optionally substituted by 1 or 2 C₁₋₆ alkyl group (e.g., methyl).

R¹ is further more preferably a 4-membered heterocyclic group or 5- or 6-membered heterocyclic group (preferably a 4-membered heterocyclic group or 5- or 6-membered non-aromatic heterocyclic group (e.g., oxetanyl, pyrrolidinyl, piperidyl, tetrahydrofuranyl, tetrahydropyranyl)), wherein the heterocyclic groups are optionally substituted by 1 to 3 substituents selected from
(a) a halogen atom (e.g., a fluorine atom),
(b) an oxo group,
(c) a C₁₋₆ alkyl group (e.g., methyl, ethyl) optionally substituted by 1 to 3 substituents selected from
   (i) a halogen atom (e.g., a fluorine atom), and
   (ii) a C₁₋₆ alkoxy group (e.g., methoxy),
(d) a C₃₋₈ cycloalkyl group (e.g., cyclopropyl),
(e) a hydroxy group,
(f) a C₁₋₆ alkoxy group (e.g., methoxy),
(g) a C₁₋₆ alkyl-carbonyl group (e.g., acetyl, propanoyl, 2-methylpropanoyl) optionally substituted by 1 to 3 substituents selected from
   (i) a halogen atom (e.g., a fluorine atom),
   (ii) a hydroxy group,
   (iii) a C₁₋₆ alkoxy group (e.g., methoxy), and
   (iv) a C₃₋₈ cycloalkyl group (e.g., cyclopropyl),
(h) a C₃₋₈ cycloalkyl-carbonyl group (e.g., cyclopropylcarbonyl) optionally substituted by 1 to 3 substituents selected from
   (i) a halogen atom (e.g., a fluorine atom),
   (ii) a cyano group, and
   (iii) a hydroxy group,
(i) a C₁₋₆ alkoxy-carbonyl group (e.g., methoxycarbonyl, ethoxycarbonyl) optionally substituted by 1 to 3 halogen atoms (e.g., a chlorine atom),
(j) a C₃₋₈ cycloalkoxy-carbonyl group (e.g., cyclopropoxycarbonyl),
(k) a C₇₋₁₆ aralkyloxy-carbonyl group (e.g., benzyloxycarbonyl),
(l) a mono- or di-C₁₋₆ alkyl-carbamoyl group (e.g., methylcarbamoyl),
(m) a C₁₋₆ alkylsulfonyl group (e.g., methylsulfonyl),
(n) a C₃₋₈ cycloalkylsulfonyl group (e.g., cyclopropylsulfonyl), and
(o) a 3- to 8-membered non-aromatic heterocyclic group (e.g., tetrahydrofuranyl).

R¹ is still more preferably a group of Formula (A):

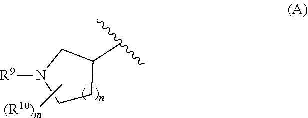

wherein
R⁹ is
(1) a hydrogen atom,
(2) a C₁₋₆ alkyl group (e.g., methyl, ethyl) optionally substituted by 1 to 3 substituents selected from
   (i) a halogen atom (e.g., a fluorine atom), and
   (ii) a C₁₋₆ alkoxy group (e.g., methoxy),
(3) a C₃₋₈ cycloalkyl group (e.g., cyclopropyl),
(4) a C₁₋₆ alkyl-carbonyl group (e.g., acetyl, propanoyl, 2-methylpropanoyl) optionally substituted by 1 to 3 substituents selected from
   (i) a halogen atom (e.g., a fluorine atom),
   (ii) a hydroxy group,
   (iii) a C₁₋₆ alkoxy group (e.g., methoxy), and
   (iv) a C₃₋₈ cycloalkyl group (e.g., cyclopropyl),
(5) a C₃₋₈ cycloalkyl-carbonyl group (e.g., cyclopropylcarbonyl) optionally substituted by 1 to 3 substituents selected from
   (i) a halogen atom (e.g., a fluorine atom),
   (ii) a cyano group, and
   (iii) a hydroxy group,
(6) a C₁₋₆ alkoxy-carbonyl group (e.g., methoxycarbonyl, ethoxycarbonyl) optionally substituted by 1 to 3 halogen atoms (e.g., a chlorine atom),
(7) a C₇₋₁₆ aralkyloxy-carbonyl group (e.g., benzyloxycarbonyl),
(8) a mono- or di-C₁₋₆ alkyl-carbamoyl group (e.g., methylcarbamoyl),
(9) a C₁₋₆ alkylsulfonyl group (e.g., methylsulfonyl),
(10) a C₃₋₈ cycloalkylsulfonyl group (e.g., cyclopropylsulfonyl), or
(11) a 3- to 8-membered non-aromatic heterocyclic group (e.g., tetrahydrofuranyl);
R¹⁰ is
(1) a halogen atom (e.g., a fluorine atom, a chlorine atom, a bromine atom),
(2) a C₁₋₆ alkyl group (e.g., methyl) optionally substituted by 1 to 3 halogen atoms (e.g., a fluorine atom), or
(3) a C₁₋₆ alkoxy group (e.g., methoxy), or
two R¹⁰ are taken together to form an oxo group;
m is 0, 1 or 2; and
n is 1 or 2.

R¹ is even more preferably a 6-membered non-aromatic heterocyclic group (preferably a 6-membered nitrogen-containing non-aromatic heterocyclic group (e.g., piperidyl)) substituted by 1 or 2 substituents selected from
(a) a halogen atom (e.g., a fluorine atom), and
(b) a C₁₋₆ alkyl group (e.g., methyl).

R¹ is particularly preferably a piperidyl group substituted by 1 or 2 substituents selected from
(a) a halogen atom (e.g., fluorine atom), and
(b) a C₁₋₂ alkyl group (e.g., methyl).

R² is a hydrogen atom, or an optionally substituted C₁₋₃ alkyl group.

In one embodiment, R² is a hydrogen atom.

In one embodiment, R² is an optionally substituted C₁₋₃ alkyl group.

As another embodiment, R² is preferably
(1) a hydrogen atom, or
(2) a C₁₋₃ alkyl group (e.g., methyl, ethyl) optionally substituted by 1 to 3 C₁₋₆ alkoxy groups (e.g., methoxy).

R² is particularly preferably a hydrogen atom.

R³, R⁴ and R⁵ are each independently a hydrogen atom, a halogen atom, an optionally substituted C₁₋₆ alkyl group, an optionally substituted C₃₋₈ cycloalkyl group, or an optionally substituted C₁₋₆ alkoxy group.

R³ is preferably a hydrogen atom, a halogen atom, an optionally substituted C₁₋₆ alkyl group, or an optionally substituted C₃₋₈ cycloalkyl group.

R³ is more preferably a hydrogen atom, a halogen atom, or an optionally substituted C₁₋₆ alkyl group.

R³ is further more preferably a hydrogen atom, a halogen atom, or a C₁₋₆ alkyl group.

R³ is still more preferably a hydrogen atom, or a C₁₋₆ alkyl group.

R³ is particularly preferably a hydrogen atom.

R⁴ is preferably a halogen atom, an optionally substituted C₁₋₆ alkyl group, or an optionally substituted C₁₋₆ alkoxy group.

R⁴ is more preferably a halogen atom, or an optionally substituted C₁₋₆ alkyl group.

R⁴ is further more preferably a halogen atom, or a C₁₋₆ alkyl group.

R⁴ is particularly preferably a C₁₋₆ alkyl group.

As another embodiment, R⁴ is preferably
(1) a hydrogen atom,
(2) a halogen atom (e.g., a fluorine atom, a chlorine atom, a bromine atom),
(3) a C₁₋₆ alkyl group (e.g., methyl, ethyl) optionally substituted by 1 to 3 halogen atoms (e.g., a fluorine atom), or
(4) a C₁₋₆ alkoxy group (e.g., methoxy) optionally substituted by 1 to 3 halogen atoms (e.g., a fluorine atom).

R⁴ is more preferably
(1) a halogen atom (e.g., a fluorine atom, a chlorine atom, a bromine atom),
(2) a C₁₋₆ alkyl group (e.g., methyl, ethyl) optionally substituted by 1 to 3 halogen atoms (e.g., a fluorine atom), or
(3) a C₁₋₆ alkoxy group (e.g., methoxy) optionally substituted by 1 to 3 halogen atoms (e.g., a fluorine atom).

R⁴ is further more preferably
(1) a halogen atom (e.g., a fluorine atom, a chlorine atom, a bromine atom), or
(2) a C₁₋₆ alkyl group (e.g., methyl, ethyl) optionally substituted by 1 to 3 halogen atoms (e.g., a fluorine atom).

R⁴ is still more preferably
(1) a halogen atom (e.g., a fluorine atom, a chlorine atom, a bromine atom), or
(2) a C₁₋₆ alkyl group (e.g., methyl, ethyl).

R⁴ is particularly preferably a C₁₋₆ alkyl group (e.g., methyl).

R⁵ is preferably a hydrogen atom, a halogen atom, an optionally substituted C₁₋₆ alkyl group, or an optionally substituted C₃₋₈ cycloalkyl group.

R⁵ is more preferably a hydrogen atom, a halogen atom, or an optionally substituted C₁₋₆ alkyl group.

R⁵ is further more preferably a hydrogen atom, a halogen atom, or a C₁₋₆ alkyl group.

R⁵ is still more preferably a hydrogen atom, or a C₁₋₆ alkyl group.

R⁵ is particularly preferably a hydrogen atom.

R⁶ is a hydrogen atom, or an optionally substituted C₁₋₆ alkyl group.

R⁶ is preferably a hydrogen atom, or a C₁₋₆ alkyl group.

R⁶ is particularly preferably a hydrogen atom.

As another embodiment, R⁶ is preferably a hydrogen atom or a C₁₋₆ alkyl group (e.g., methyl).

R⁶ is particularly preferably a hydrogen atom.

Preferable embodiment of a compound of Formula (I) includes the following compounds.

### [Compound A]

A compound of Formula (I) wherein
X is S or NR⁷;
Y is CR⁸ or N;
R¹ is
(1) a 4-membered heterocyclic group or 5- or 6-membered heterocyclic group (preferably a 4-membered heterocyclic group or 5- or 6-membered non-aromatic heterocyclic group (e.g., oxetanyl, pyrrolidinyl, piperidyl, tetrahydrofuranyl, tetrahydropyranyl)), wherein the heterocyclic groups are optionally substituted by 1 to 3 substituents selected from
   (a) a halogen atom (e.g., a fluorine atom),
   (b) an oxo group,
   (c) a C₁₋₆ alkyl group (e.g., methyl, ethyl) optionally substituted by 1 to 3 substituents selected from
      (i) a halogen atom (e.g., a fluorine atom), and
      (ii) a C₁₋₆ alkoxy group (e.g., methoxy),
   (d) a C₃₋₈ cycloalkyl group (e.g., cyclopropyl),
   (e) a hydroxy group,
   (f) a C₁₋₆ alkoxy group (e.g., methoxy),
   (g) a C₁₋₆ alkyl-carbonyl group (e.g., acetyl, propanoyl, 2-methylpropanoyl) optionally substituted by 1 to 3 substituents selected from
      (i) a halogen atom (e.g., a fluorine atom),
      (ii) a hydroxy group,
      (iii) a C₁₋₆ alkoxy group (e.g., methoxy), and
      (iv) a C₃₋₈ cycloalkyl group (e.g., cyclopropyl),
   (h) a C₃₋₈ cycloalkyl-carbonyl group (e.g., cyclopropylcarbonyl) optionally substituted by 1 to 3 substituents selected from
      (i) a halogen atom (e.g., a fluorine atom),
      (ii) a cyano group, and
      (iii) a hydroxy group,
   (i) a C₁₋₆ alkoxy-carbonyl group (e.g., methoxycarbonyl, ethoxycarbonyl) optionally substituted by 1 to 3 halogen atoms (e.g., a chlorine atom),
   (j) a C₃₋₈ cycloalkoxy-carbonyl group (e.g., cyclopropoxycarbonyl),
   (k) a C₇₋₁₆ aralkyloxy-carbonyl group (e.g., benzyloxycarbonyl),
   (l) a mono- or di-C₁₋₆ alkyl-carbamoyl group (e.g., methylcarbamoyl),
   (m) a C₁₋₆ alkylsulfonyl group (e.g., methylsulfonyl),
   (n) a C₃₋₈ cycloalkylsulfonyl group (e.g., cyclopropylsulfonyl), and
   (o) a 3- to 8-membered non-aromatic heterocyclic group (e.g., tetrahydrofuranyl),
(2) a C₁₋₆ alkyl group (e.g., methyl, ethyl, propyl, isopropyl, butyl, isobutyl, sec-butyl, isopentyl) optionally substituted by 1 to 3 substituents selected from
   (a) a hydroxy group,
   (b) a C₁₋₆ alkoxy group (e.g., methoxy) optionally substituted by 1 to 3 halogen atoms (e.g., fluorine atom),
   (c) a C₃₋₈ cycloalkyl group (e.g., cyclopropyl, cyclobutyl) optionally substituted by 1 to 3 halogen atoms (e.g., a fluorine atom),
   (d) a mono- or di-C₁₋₆ alkylamino group (e.g., dimethylamino),
   (e) a C₁₋₆ alkyl-carbonylamino group (e.g., acetylamino),
   (f) a 3- to 8-membered non-aromatic heterocyclic group (e.g., oxetanyl, morpholinyl, tetrahydrofuranyl) optionally substituted by a hydroxy group,
   (g) a halogen atom (e.g., fluorine atom), and
   (h) a 5- to 14-membered aromatic heterocyclyl group (e.g., pyridyl), or
(3) a C₃₋₁₀ cycloalkyl group (the C₃₋₁₀ cycloalkyl group may be a spiro ring group or a bridged spiro ring group, e.g., cyclopropyl, cyclobutyl, cyclopentyl, cyclohexyl, spiro[3.3]heptanyl, bicyclo[1.1.1]pentyl, bicyclo[2.2.1]heptanyl, adamantyl) optionally substituted by 1 to 3 substituents selected from
   (a) a hydroxy group,
   (b) a C₁₋₆ alkyl group (e.g., methyl) optionally substituted by 1 to 3 halogen atoms (e.g., fluorine atom), and
   (c) a C₁₋₆ alkoxy group (e.g., methoxy) optionally substituted by 1 to 3 halogen atoms (e.g., fluorine atom);
R² is
(1) a hydrogen atom, or
(2) a C₁₋₃ alkyl group (e.g., methyl, ethyl) optionally substituted by 1 to 3 C₁₋₆ alkoxy groups (e.g., methoxy);
R³ is a hydrogen atom;
R⁴ is
(1) a hydrogen atom,
(2) a halogen atom (e.g., a fluorine atom, a chlorine atom, a bromine atom),
(3) a C₁₋₆ alkyl group (e.g., methyl, ethyl) optionally substituted by 1 to 3 halogen atoms (e.g., a fluorine atom), or
(4) a C₁₋₆ alkoxy group (e.g., methoxy) optionally substituted by 1 to 3 halogen atoms (e.g., a fluorine atom);
R⁵ is a hydrogen atom;
R⁶ is a hydrogen atom or a C₁₋₆ alkyl group (e.g., methyl);
R⁷ is a C₁₋₆ alkyl group (e.g., methyl); and
R⁸ is
(1) a hydrogen atom,
(2) a halogen atom (e.g., a chlorine atom),
(3) a C₁₋₆ alkyl group (e.g., methyl, ethyl, isopropyl) optionally substituted by 1 to 3 substituents selected from (i) halogen atoms (e.g., a fluorine atom) and (ii) a C₁₋₆ alkoxy group (e.g., methoxy) optionally substituted by 1 to 3 halogen atoms (e.g., a fluorine atom),
(4) a C₃₋₈ cycloalkyl group (e.g., cyclopropyl) optionally substituted by 1 to 3 halogen atoms (e.g., a fluorine atom), or
(5) a C₁₋₆ alkoxy group (e.g., methoxy) optionally substituted by 1 to 3 halogen atoms (e.g., a fluorine atom).

### [Compound B-1]

A compound of Formula (I) wherein
R¹ is
(1) a 4-membered heterocyclic group or 5- or 6-membered heterocyclic group (preferably a 4-membered heterocyclic group or 5- or 6-membered non-aromatic heterocyclic group (e.g., oxetanyl, pyrrolidinyl, piperidyl, tetrahydrofuranyl, tetrahydropyranyl)), wherein the heterocyclic groups are optionally substituted by 1 to 3 substituents selected from
   (a) a halogen atom (e.g., a fluorine atom),
   (b) an oxo group,
   (c) a C₁₋₆ alkyl group (e.g., methyl, ethyl) optionally substituted by 1 to 3 substituents selected from
      (i) a halogen atom (e.g., a fluorine atom), and
      (ii) a C₁₋₆ alkoxy group (e.g., methoxy),
   (d) a C₃₋₈ cycloalkyl group (e.g., cyclopropyl),
   (e) a hydroxy group,
   (f) a C₁₋₆ alkoxy group (e.g., methoxy),
   (g) a C₁₋₆ alkyl-carbonyl group (e.g., acetyl, propanoyl, 2-methylpropanoyl) optionally substituted by 1 to 3 substituents selected from
      (i) a halogen atom (e.g., a fluorine atom),
      (ii) a hydroxy group,
      (iii) a C₁₋₆ alkoxy group (e.g., methoxy), and
      (iv) a C₃₋₈ cycloalkyl group (e.g., cyclopropyl),
   (h) a C₃₋₈ cycloalkyl-carbonyl group (e.g., cyclopropylcarbonyl) optionally substituted by 1 to 3 substituents selected from
      (i) a halogen atom (e.g., a fluorine atom),
      (ii) a cyano group, and
      (iii) a hydroxy group,
   (i) a C₁₋₆ alkoxy-carbonyl group (e.g., methoxycarbonyl, ethoxycarbonyl) optionally substituted by 1 to 3 halogen atoms (e.g., a chlorine atom),
   (j) a C₃₋₈ cycloalkoxy-carbonyl group (e.g., cyclopropoxycarbonyl),
   (k) a C₇₋₁₆ aralkyloxy-carbonyl group (e.g., benzyloxycarbonyl),
   (l) a mono- or di-C₁₋₆ alkyl-carbamoyl group (e.g., methylcarbamoyl),
   (m) a C₁₋₆ alkylsulfonyl group (e.g., methylsulfonyl),
   (n) a C₃₋₈ cycloalkylsulfonyl group (e.g., cyclopropylsulfonyl), and
   (o) a 3- to 8-membered non-aromatic heterocyclic group (e.g., tetrahydrofuranyl),
(2) a C₁₋₆ alkyl group (e.g., methyl, ethyl, propyl, butyl, isobutyl, sec-butyl, isopentyl) substituted by 1 to 3 substituents selected from
   (a) a hydroxy group,
   (b) a C₁₋₆ alkoxy group (e.g., methoxy) optionally substituted by 1 to 3 halogen atoms (e.g., a fluorine atom),
   (c) a C₃₋₈ cycloalkyl group (e.g., cyclopropyl, cyclobutyl) substituted by 1 to 3 halogen atoms (e.g., a fluorine atom),
   (d) a mono- or di-C₁₋₆ alkylamino group (e.g., dimethylamino),
   (e) a C₁₋₆ alkyl-carbonylamino group (e.g., acetylamino),
   (f) a 3- to 8-membered non-aromatic heterocyclic group (e.g., oxetanyl, morpholinyl, tetrahydrofuranyl) optionally substituted by a hydroxy group, and
   (g) a halogen atom (e.g., fluorine atom), and
   (h) a 5- to 14-membered aromatic heterocyclyl group (e.g., pyridyl), or
(3) a C₃₋₁₀ cycloalkyl group (the C₃₋₁₀ cycloalkyl group may be a spiro ring group or a bridged spiro ring group, e.g., cyclopropyl, cyclobutyl, cyclopentyl, cyclohexyl, spiro[3.3]heptanyl, bicyclo[1.1.1]pentyl, bicyclo[2.2.1]heptanyl, adamantyl) optionally substituted by 1 to 3 substituents selected by
   (a) a hydroxy group,
   (b) a C₁₋₆ alkyl group (e.g., methyl) optionally substituted by 1 to 3 halogen atoms (e.g., a fluorine atom), and
   (c) a C₁₋₆ alkoxy group (e.g., methoxy) optionally substituted by 1 to 3 halogen atoms (e.g., a fluorine atom); and
the other variables are as defined in [Compound A].

### [Compound B-2]

A compound of Formula (I) wherein
R¹ is a 4-membered heterocyclic group or 5- or 6-membered heterocyclic group (preferably a 4-membered heterocyclic group or 5- or 6-membered non-aromatic heterocyclic group (e.g., oxetanyl, pyrrolidinyl, piperidyl, tetrahydrofuranyl, tetrahydropyranyl)), wherein the heterocyclic groups are optionally substituted by 1 to 3 substituents selected from
   (a) a halogen atom (e.g., a fluorine atom),
   (b) an oxo group,
   (c) a C₁₋₆ alkyl group (e.g., methyl, ethyl) optionally substituted by 1 to 3 substituents selected from
      (i) a halogen atom (e.g., a fluorine atom), and
      (ii) a C₁₋₆ alkoxy group (e.g., methoxy),
   (d) a C₃₋₈ cycloalkyl group (e.g., cyclopropyl),
   (e) a hydroxy group,
   (f) a C₁₋₆ alkoxy group (e.g., methoxy),
   (g) a C₁₋₆ alkyl-carbonyl group (e.g., acetyl, propanoyl, 2-methylpropanoyl) optionally substituted by 1 to 3 substituents selected from
      (i) a halogen atom (e.g., a fluorine atom),
      (ii) a hydroxy group,
      (iii) a C₁₋₆ alkoxy group (e.g., methoxy), and
      (iv) a C₃₋₈ cycloalkyl group (e.g., cyclopropyl),
   (h) a C₃₋₈ cycloalkyl-carbonyl group (e.g., cyclopropylcarbonyl) optionally substituted by 1 to 3 substituents selected from
      (i) a halogen atom (e.g., a fluorine atom),
      (ii) a cyano group, and
      (iii) a hydroxy group,
   (i) a C₁₋₆ alkoxy-carbonyl group (e.g., methoxycarbonyl, ethoxycarbonyl) optionally substituted by 1 to 3 halogen atoms (e.g., a chlorine atom),
   (j) a C₃₋₈ cycloalkoxy-carbonyl group (e.g., cyclopropoxycarbonyl)
   (k) a C₇₋₁₆ aralkyloxy-carbonyl group (e.g., benzyloxycarbonyl),
   (l) a mono- or di-C₁₋₆ alkyl-carbamoyl group (e.g., methylcarbamoyl),
   (m) a C₁₋₆ alkylsulfonyl group (e.g., methylsulfonyl),
   (n) a C₃₋₈ cycloalkylsulfonyl group (e.g., cyclopropylsulfonyl), and
   (o) a 3- to 8-membered non-aromatic heterocyclic group (e.g., tetrahydrofuranyl); and
the other variables are as defined in [Compound A].

### [Compound B-3]

A compound of Formula (I) wherein
R¹ is a group of Formula (A):

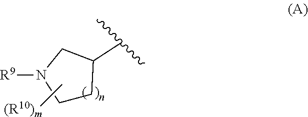

wherein
R⁹ is
(1) a hydrogen atom,
(2) a C₁₋₆ alkyl group (e.g., methyl, ethyl) optionally substituted by 1 to 3 substituents selected from
   (i) a halogen atom (e.g., a fluorine atom), and
   (ii) a C₁₋₆ alkoxy group (e.g., methoxy),
(3) a C₃₋₈ cycloalkyl group (e.g., cyclopropyl),
(4) a C₁₋₆ alkyl-carbonyl group (e.g., acetyl, propanoyl, 2-methylpropanoyl) optionally substituted by 1 to 3 substituents selected from
   (i) a halogen atom (e.g., a fluorine atom),
   (ii) a hydroxy group,
   (iii) a C₁₋₆ alkoxy group (e.g., methoxy), and
   (iv) a C₃₋₈ cycloalkyl group (e.g., cyclopropyl),
(5) a C₃₋₈ cycloalkyl-carbonyl group (e.g., cyclopropylcarbonyl) optionally substituted by 1 to 3 substituents selected from
   (i) a halogen atom (e.g., a fluorine atom),
   (ii) a cyano group, and
   (iii) a hydroxy group,
(6) a C₁₋₆ alkoxy-carbonyl group (e.g., methoxycarbonyl, ethoxycarbonyl) optionally substituted by 1 to 3 halogen atoms (e.g., a chlorine atom),
(7) a C₇₋₁₆ aralkyloxy-carbonyl group (e.g., benzyloxycarbonyl),
(8) a mono- or di-C₁₋₆ alkyl-carbamoyl group (e.g., methylcarbamoyl),
(9) a C₁₋₆ alkylsulfonyl group (e.g., methylsulfonyl),
(10) a C₃₋₈ cycloalkylsulfonyl group (e.g., cyclopropylsulfonyl), or
(11) a 3- to 8-membered non-aromatic heterocyclic group (e.g., tetrahydrofuranyl);
R¹⁰ is
(1) a halogen atom (e.g., a fluorine atom, a chlorine atom, a bromine atom),
(2) a C₁₋₆ alkyl group (e.g., methyl) optionally substituted by 1 to 3 halogen atoms (e.g., a fluorine atom), or
(3) a C₁₋₆ alkoxy group (e.g., methoxy), or
two R¹⁰ are taken together to form an oxo group;
m is 0, 1 or 2; and
n is 1 or 2; and
the other variables are as defined in [Compound A].

### [Compound C-1]

A compound of Formula (I) wherein
X is S; and
the other variables are as defined in [Compound A], [Compound B-1], [Compound B-2] or [Compound B-3].

### [Compound C-2]

A compound of Formula (I) wherein
X is NR⁷ wherein R⁷ is a C₁₋₆ alkyl group (e.g., methyl); and
   the other variables are as defined in [Compound A], [Compound B-1], [Compound B-2] or [Compound B-3]. [Compound D-1]

A compound of Formula (I) wherein
R² is a hydrogen atom; and
the other variables are as defined in [Compound A], [Compound B-1], [Compound B-2], [Compound B-3], [Compound C-1] or [Compound C-2].

### [Compound D-2]

A compound of Formula (I) wherein
R² is a C₁₋₃ alkyl group (e.g., methyl, ethyl) optionally substituted by 1 to 3 C₁₋₆ alkoxy groups (e.g., methoxy); and
the other variables are as defined in [Compound A], [Compound B-1], [Compound B-2], [Compound B-3], [Compound C-1] or [Compound C-2].

### [Compound E-1]

A compound of Formula (I) wherein
Y is CR⁸ wherein R⁸ is
(1) a hydrogen atom,
(2) a halogen atom (e.g., a chlorine atom),
(3) a C₁₋₆ alkyl group (e.g., methyl, ethyl, isopropyl) optionally substituted by 1 to 3 substituents selected from (i) halogen atoms (e.g., a fluorine atom) and (ii) a C₁₋₆ alkoxy group (e.g., methoxy),
(4) a C₃₋₈ cycloalkyl group (e.g., cyclopropyl) optionally substituted by 1 to 3 halogen atoms (e.g., a fluorine atom), or
(5) a C₁₋₆ alkoxy group (e.g., methoxy) optionally substituted by 1 to 3 halogen atoms (e.g., a fluorine atom); and
the other variables are as defined in [Compound A], [Compound B-1], [Compound B-2], [Compound B-3], [Compound C-1], [Compound C-2], [Compound D-1] or [Compound D-2].

### [Compound E-2]

A compound of Formula (I) wherein
Y is CR⁸ wherein R⁸ is
(1) a halogen atom (e.g., a chlorine atom),
(2) a C₁₋₆ alkyl group (e.g., methyl, ethyl, isopropyl) optionally substituted by 1 to 3 substituents selected from (i) halogen atoms (e.g., a fluorine atom) and (ii) a C₁₋₆ alkoxy group (e.g., methoxy),
(3) a C₃₋₈ cycloalkyl group (e.g., cyclopropyl) optionally substituted by 1 to 3 halogen atoms (e.g., a fluorine atom), or
(4) a C₁₋₆ alkoxy group (e.g., methoxy) optionally substituted by 1 to 3 halogen atoms (e.g., a fluorine atom); and
the other variables are as defined in [Compound A], [Compound B-1], [Compound B-2], [Compound B-3], [Compound C-1], [Compound C-2], [Compound D-1] or [Compound D-2].

### [Compound E-3]

A compound of Formula (I) wherein
Y is CR⁸ wherein R⁸ is a C₁₋₆ alkyl group (e.g., methyl, ethyl, isopropyl) optionally substituted by 1 to 3 substituents selected from (i) halogen atoms (e.g., a fluorine atom) and (ii) a C₁₋₆ alkoxy group (e.g., methoxy); and
the other variables are as defined in [Compound A], [Compound B-1], [Compound B-2], [Compound B-3], [Compound C-1], [Compound C-2], [Compound D-1] or [Compound D-2].

### [Compound F]

A compound of Formula (I) wherein
R⁴ is
(1) a halogen atom (e.g., a fluorine atom, a chlorine atom, a bromine atom),
(2) a C₁₋₆ alkyl group (e.g., methyl, ethyl) optionally substituted by 1 to 3 halogen atoms (e.g., a fluorine atom), or
(3) a C₁₋₆ alkoxy group (e.g., methoxy) optionally substituted by 1 to 3 halogen atoms (e.g., a fluorine atom); and
the other variables are as defined in [Compound A], [Compound B-1], [Compound B-2], [Compound B-3], [Compound C-1], [Compound C-2], [Compound D-1], [Compound D-2], [Compound E-1], [Compound E-2] or [Compound E-3].

### [Compound G-1]

A compound of Formula (I) wherein
X is S;
Y is CR⁸ wherein R⁸ is a C₁₋₆ alkyl group (e.g., methyl, ethyl);
R¹ is a 6-membered non-aromatic heterocyclic group (e.g., piperidyl) substituted by 1 or 2 substituents selected from
   (a) a halogen atom (e.g., a fluorine atom, a chlorine atom, a bromine atom), and
   (b) a C₁₋₆ alkyl group (e.g., methyl);
R² is a hydrogen atom;
R³ is a hydrogen atom;
R⁴ is a C₁₋₆ alkyl group (e.g., methyl);
R⁵ is a hydrogen atom; and
R⁶ is a hydrogen atom.

### [Compound G-2]

A compound of Formula (I) wherein
X is S;
Y is CR⁸ wherein R⁸ is a C₁₋₆ alkyl group (e.g., methyl, ethyl);
R¹ is a 6-membered nitrogen-containing non-aromatic heterocyclic group (e.g., piperidyl) substituted by 1 or 2 substituents selected from
   (a) a halogen atom (e.g., a fluorine atom, a chlorine atom, a bromine atom), and
   (b) a C₁₋₆ alkyl group (e.g., methyl);
R² is a hydrogen atom;
R³ is a hydrogen atom;
R⁴ is a C₁₋₆ alkyl group (e.g., methyl);
R⁵ is a hydrogen atom; and
R⁶ is a hydrogen atom.

### [Compound G-3]

A compound of Formula (I) wherein
X is S;
Y is CR⁸ wherein R⁸ is a C₁₋₆ alkyl group (e.g., methyl, ethyl);
R¹ is a piperidyl group substituted by 1 or 2 substituents selected from
   (a) a halogen atom (e.g., a fluorine atom, a chlorine atom, a bromine atom), and
   (b) a C₁₋₆ alkyl group (e.g., methyl);
R² is a hydrogen atom;
R³ is a hydrogen atom;
R⁴ is a C₁₋₆ alkyl group (e.g., methyl);
R⁵ is a hydrogen atom; and
R⁶ is a hydrogen atom.

### [Compound G-4]

A compound of Formula (I) wherein
X is S;
Y is CR⁸ wherein R⁸ is a C₁₋₆ alkyl group (e.g., methyl, ethyl);
R¹ is a piperidyl group substituted by 1 or 2 substituents selected from
   (a) a halogen atom, and
   (b) a C₁₋₂ alkyl group (e.g., methyl);
R² is a hydrogen atom;
R³ is a hydrogen atom;
R⁴ is a C₁₋₂ alkyl group (e.g., methyl);
R⁵ is a hydrogen atom; and
R⁶ is a hydrogen atom.

### [Compound H-1]

A compound of Formula (I) wherein
R¹ is as defined in [Compound B-1];
Y is as defined in [Compound E-1];
R⁴ is as defined in [Compound F]; and
the other variables are as defined in [Compound A].

### [Compound H-2]

A compound of Formula (I) wherein
R¹ is as defined in [Compound B-2];
Y is as defined in [Compound E-1];
R⁴ is as defined in [Compound F]; and
the other variables are as defined in [Compound A].

### [Compound H-3]

A compound of Formula (I) wherein
R¹ is as defined in [Compound B-3];
Y is as defined in [Compound E-1];
R⁴ is as defined in [Compound F]; and
the other variables are as defined in [Compound A].

### [Compound H-4]

A compound of Formula (I) wherein
R¹ is as defined in [Compound B-1];
Y is as defined in [Compound E-2];
R⁴ is as defined in [Compound F]; and
the other variables are as defined in [Compound A].

### [Compound H-5]

A compound of Formula (I) wherein
R¹ is as defined in [Compound B-2];
Y is as defined in [Compound E-2];
R⁴ is as defined in [Compound F]; and
the other variables are as defined in [Compound A].

### [Compound H-6]

A compound of Formula (I) wherein
R¹ is as defined in [Compound B-3];
Y is as defined in [Compound E-2];
R⁴ is as defined in [Compound F]; and
the other variables are as defined in [Compound A].

### [Compound H-7]

A compound of Formula (I) wherein
R¹ is as defined in [Compound B-1];
Y is as defined in [Compound E-3];
R⁴ is as defined in [Compound F]; and
the other variables are as defined in [Compound A].

### [Compound H-8]

A compound of Formula (I) wherein
R¹ is as defined in [Compound B-2];
Y is as defined in [Compound E-3];
R⁴ is as defined in [Compound F]; and
the other variables are as defined in [Compound A].

### [Compound H-9]

A compound of Formula (I) wherein
R¹ is as defined in [Compound B-3];
Y is as defined in [Compound E-3];
R⁴ is as defined in [Compound F]; and
the other variables are as defined in [Compound A].

### [Compound I-1]

A compound of Formula (I) wherein
X is S;
R¹ is as defined in [Compound B-1];
Y is as defined in [Compound E-1];
R⁴ is as defined in [Compound F]; and
the other variables are as defined in [Compound A].

### [Compound I-2]

A compound of Formula (I) wherein
X is S;
R¹ is as defined in [Compound B-2];
Y is as defined in [Compound E-1];
R⁴ is as defined in [Compound F]; and
the other variables are as defined in [Compound A].

### [Compound I-3]

A compound of Formula (I) wherein
X is S;
R¹ is as defined in [Compound B-3];
Y is as defined in [Compound E-1];
R⁴ is as defined in [Compound F]; and
the other variables are as defined in [Compound A].

### [Compound I-4]

A compound of Formula (I) wherein
X is S;
R¹ is as defined in [Compound B-1];
Y is as defined in [Compound E-2];
R⁴ is as defined in [Compound F]; and
the other variables are as defined in [Compound A].

### [Compound I-5]

A compound of Formula (I) wherein
X is S;
R¹ is as defined in [Compound B-2];
Y is as defined in [Compound E-2];
R⁴ is as defined in [Compound F]; and
the other variables are as defined in [Compound A].

### [Compound I-6]

A compound of Formula (I) wherein
X is S;
R¹ is as defined in [Compound B-3];
Y is as defined in [Compound E-2];
R⁴ is as defined in [Compound F]; and
the other variables are as defined in [Compound A].

### [Compound I-7]

A compound of Formula (I) wherein
X is S;
R¹ is as defined in [Compound B-1];
Y is as defined in [Compound E-3];
R⁴ is as defined in [Compound F]; and
the other variables are as defined in [Compound A].

### [Compound I-8]

A compound of Formula (I) wherein
X is S;
R¹ is as defined in [Compound B-2];
Y is as defined in [Compound E-3];
R⁴ is as defined in [Compound F]; and
the other variables are as defined in [Compound A].

### [Compound I-9]

A compound of Formula (I) wherein
X is S;
R¹ is as defined in [Compound B-3];
Y is as defined in [Compound E-3];
R⁴ is as defined in [Compound F]; and
the other variables are as defined in [Compound A].

### [Compound J-1]

A compound of Formula (I) wherein
R³ is a hydrogen atom;
R⁵ is a hydrogen atom;
R⁶ is a hydrogen atom or a C₁₋₆ alkyl group (e.g., methyl); and
the other variables are as defined in [Compound A], [Compound B-1], [Compound B-2], [Compound B-3], [Compound C-1], [Compound C-2], [Compound D-1] or [Compound D-2].

### [Compound K]

A compound of Formula (I) which is selected from 2-(5-{[(3R,5R)-5-fluoro-1-methylpiperidin-3-yl]amino}-2H-pyrazolo[3,4-d][1,3]thiazol-2-yl)-3,5-dimethylphenol, or a solvate thereof, or a hydrate thereof, 3,5-dimethyl-2-(5-{[(3R)-1-methylpiperidin-3-yl]amino}-2H-pyrazolo[3,4-d][1,3]thiazol-2-yl)phenol, or a pharmaceutically acceptable salt thereof, or a solvate thereof, or a hydrate thereof, and 3-ethyl-2-(5-{[(3R,5R)-5-fluoro-1-methylpiperidin-3-yl]amino}-2H-pyrazolo[3,4-d][1,3]thiazol-2-yl)-5-methylphenol, or a pharmaceutically acceptable salt thereof, or a solvate thereof, or a hydrate thereof.

### [Compound L-1]

2-(5-{[(3R,5R)-5-fluoro-1-methylpiperidin-3-yl]amino}-2H-pyrazolo[3,4-d][1,3]thiazol-2-yl)-3,5-dimethylphenol, or a pharmaceutically acceptable salt thereof, or a solvate thereof, or a hydrate thereof (Example 3)

### [Compound L-2]

3,5-dimethyl-2-(5-{[(3R)-1-methylpiperidin-3-yl]amino}-2H-pyrazolo[3,4-d][1,3]thiazol-2-yl)phenol, or a pharmaceutically acceptable salt thereof, or a solvate thereof, or a hydrate thereof (Example 4)

### [Compound L-3]

3-ethyl-2-(5-{[(3R,5R)-5-fluoro-1-methylpiperidin-3-yl]amino}-2H-pyrazolo[3,4-d][1,3]thiazol-2-yl)-5-methylphenol, or a pharmaceutically acceptable salt thereof, or a solvate thereof, or a hydrate thereof (Example 20)

Specific examples of a compound of Formula (I) include the compounds of Examples 1 to 181.

Compounds of Formula (I) include embodiments described in the preceding paragraphs and compounds specifically named in the examples, may exist as salts, complexes, solvates, hydrates, and liquid crystals. Likewise, compounds of Formula (I) that are salts may exist as complexes, solvates, hydrates, and liquid crystals.

Compounds of Formula (I) may form pharmaceutically acceptable complexes, salts, solvates and hydrates. These salts include acid addition salts (including di-acids) and base salts. Pharmaceutically acceptable acid addition salts include salts derived from inorganic acids such as hydrochloric acid, nitric acid, phosphoric acid, sulfuric acid, hydrobromic acid, hydroiodic acid, hydrofluoric acid, and phosphorous acids, as well nontoxic salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts include acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate, hydrogen phosphate, dihydrogen phosphate, pyroglutamate, saccharate, stearate, succinate, tannate, tartrate, tosylate, trifluoroacetate and xinofoate salts.

Pharmaceutically acceptable base salts include salts derived from bases, including metal cations, such as an alkali or alkaline earth metal cation, as well as amines. Examples of suitable metal cations include sodium, potassium, magnesium, calcium, zinc, and aluminum. Examples of suitable amines include arginine, N,N′-dibenzylethylenediamine, chloroprocaine, choline, diethylamine, diethanolamine, dicyclohexylamine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., J. Pharm. Sci. (1977) 66:1-19; see also Stahl and Wermuth, Handbook of Pharmaceutical Salts: Properties, Selection, and Use (2002).

Pharmaceutically acceptable salts may be prepared using various methods. For example, a compound of Formula (I) may be reacted with an appropriate acid or base to give the desired salt. Alternatively, a precursor of the compound of Formula (I) may be reacted with an acid or base to remove an acid- or base-labile protecting group or to open a lactone or lactam group of the precursor. Additionally, a salt of the compound of Formula (I) may be converted to another salt (or free form) through treatment with an appropriate acid or base or through contact with an ion exchange resin. Following reaction, the salt may be isolated by filtration if it precipitates from solution, or by evaporation to recover the salt. The degree of ionization of the salt may vary from completely ionized to almost non-ionized.

Compounds of Formula (I) may exist in a continuum of solid states ranging from fully amorphous to fully crystalline. The term “amorphous” refers to a state in which the material lacks long range order at the molecular level and, depending upon temperature, may exhibit the physical properties of a solid or a liquid. Typically, such materials do not give distinctive X-ray diffraction patterns and, while exhibiting the properties of a solid, are more formally described as a liquid. Upon heating, a change from solid to liquid properties occurs which is characterized by a change of state, typically second order (“glass transition”). The term “crystalline” refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (“melting point”).

Compounds of Formula (I) may also exist in unsolvated and solvated forms. The term “solvate” describes a molecular complex comprising the compound and one or more pharmaceutically acceptable solvent molecules (e.g., ethanol) which is other than water. The term “hydrate” means a solvate in which the solvent is water. Pharmaceutically acceptable solvates include those in which the solvent may be isotopically substituted (e.g., D₂O, acetone-d₆, DMSO-d₆).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will typically be observed.

Compounds of Formula (I) may also exist as multi-component complexes (other than salts and solvates) in which the compound (drug) and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun. (2004) 17:1889-1896. For a general review of multi-component complexes, see J. K. Haleblian, J. Pharm. Sci. (1975) 64(8):1269-88.

When subjected to suitable conditions, compounds of Formula (I) may exist in a mesomorphic state (mesophase or liquid crystal). The mesomorphic state lies between the true crystalline state and the true liquid state (either melt or solution). Mesomorphism arising as the result of a change in temperature is described as “thermotropic” and mesomorphism resulting from the addition of a second component, such as water or another solvent, is described as “lyotropic.” Compounds that have the potential to form lyotropic mesophases are described as “amphiphilic” and include molecules which possess a polar ionic moiety (e.g., —COO⁻Na⁺, —COO⁻K⁺, —SO₃⁻Na⁺) or polar non-ionic moiety (such as —N⁻N⁺(CH₃)₃). See, e.g., N. H. Hartshorne and A. Stuart, Crystals and the Polarizing Microscope (4th ed, 1970).

Each compound of Formula (I) may exist as polymorphs, stereoisomers, tautomers, or some combination thereof, may be isotopically-labeled, may result from the administration of a prodrug, or form a metabolite following administration.

“Prodrugs” refer to compounds having little or no pharmacological activity that can, when metabolized in vivo, undergo conversion to compounds having desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of compounds of Formula (I) having carboxylic acid, hydroxy, or amino functional groups, respectively. For further discussions of prodrugs, see e.g., T. Higuchi and V. Stella “Pro-drugs as Novel Delivery Systems,” ACS Symposium Series 14 (1975) and E. B. Roche ed., Bioreversible Carriers in Drug Design (1987).

“Metabolites” refer to compounds formed in vivo upon administration of pharmacologically active compounds. Examples include hydroxymethyl, hydroxy, secondary amino, primary amino, phenol, and carboxylic acid derivatives of compounds of Formula (I) having methyl, alkoxy, tertiary amino, secondary amino, phenyl, and amide groups, respectively.

Compounds of Formula (I) may exist as stereoisomers that result from the presence of one or more stereogenic centers, one or more double bonds, or both. The stereoisomers may be pure, substantially pure, or mixtures. Such stereoisomers may also result from acid addition or base salts in which the counter-ion is optically active, for example, when the counter-ion is D-lactate or L-lysine.

Compounds of Formula (I) may exist as tautomers, which are isomers resulting from tautomerization. Tautomeric isomerism includes, for example, imine-enamine, keto-enol, oxime-nitroso, and amide-imidic acid tautomerism.

Compounds of Formula (I) may exhibit more than one type of isomerism.

Geometrical (cis/trans) isomers may be separated by conventional techniques such as chromatography and fractional crystallization.

Conventional techniques for preparing or isolating a compound having a specific stereochemical configuration include chiral synthesis from a suitable optically pure precursor or resolution of the racemate (or the racemate of a salt or derivative) using, for example, chiral high-pressure liquid chromatography (HPLC). Alternatively, the racemate (or a racemic precursor) may be reacted with a suitable optically active compound, for example, an alcohol, or, in the case where the compound of Formula (I) contains an acidic or basic moiety, an acid or base such as tartaric acid or 1-phenylethylamine. The resulting diastereomeric mixture may be separated by chromatography, fractional crystallization, etc., and the appropriate diastereoisomer converted to the compound having the requisite stereochemical configuration. For a further discussion of techniques for separating stereoisomers, see E. L. Eliel and S. H. Wilen, Stereochemistry of Organic Compounds (1994).

Compounds of Formula (I) may possess isotopic variations, in which at least one atom is replaced by an atom having the same atomic number, but an atomic mass different from the atomic mass usually found in nature. Isotopes suitable for inclusion in compounds of Formula (I) include, for example, isotopes of hydrogen, such as ²H and ³H; isotopes of carbon, such as ¹¹C, ¹³C and ¹⁴C; isotopes of nitrogen, such as ¹³N and ¹⁵N; isotopes of oxygen, such as ¹⁵O, ¹⁷O and ¹⁸O; isotopes of sulfur, such as ³⁵S; isotopes of fluorine, such as ¹⁸F; isotopes of chlorine, such as ³⁶Cl, and isotopes of iodine, such as ¹²³I and ¹²⁵I. Use of isotopic variations (e.g., deuterium, ²H) may afford certain therapeutic advantages resulting from greater metabolic stability, for example, increased in vivo half-life or reduced dosage requirements. Additionally, certain isotopic variations of the disclosed compounds may incorporate a radioactive isotope (e.g., tritium, ³H, or ¹⁴C), which may be useful in drug and/or substrate tissue distribution studies. Substitution with positron emitting isotopes, such as ¹¹C, ¹⁸F, ¹⁵O and ¹³N, may be useful in Positron Emission Topography (PET) studies for examining substrate receptor occupancy. Isotopically-labeled compounds may be prepared by processes analogous to those described elsewhere in the disclosure using an appropriate isotopically-labeled reagent in place of a non-labeled reagent.

Compound (I) may be prepared using the techniques described below. Some of the methods and examples may omit details of common reactions, including oxidations, reductions, and so on, separation techniques (extraction, evaporation, precipitation, chromatography, filtration, trituration, crystallization, and the like), and analytical procedures, which are known to persons of ordinary skill in the art of organic chemistry. The details of such reactions and techniques can be found in several treatises, including Richard Larock, Comprehensive Organic Transformations (1999), and the multi-volume series edited by Michael B. Smith and others, Compendium of Organic Synthetic Methods (1974 et seq.). Starting materials and reagents may be obtained from commercial sources or may be prepared using literature methods. Some of the reaction schemes may omit minor products resulting from chemical transformations (e.g., an alcohol from the hydrolysis of an ester, CO₂ from the decarboxylation of a di-acid, etc.). In addition, in some instances, reaction intermediates may be used in subsequent steps without isolation or purification (i.e., in situ).

In the methods and examples below, certain compounds may be prepared using protecting groups, which prevent undesirable chemical reaction at otherwise reactive sites. Protecting groups may also be used to enhance solubility or otherwise modify physical properties of a compound. For a discussion of protecting group strategies, a description of materials and methods for installing and removing protecting groups, and a compilation of useful protecting groups for common functional groups, including amines, carboxylic acids, alcohols, ketones, aldehydes, and so on, see T. W. Greene and P. G. Wuts, Protecting Groups in Organic Chemistry (1999) and P. Kocienski, Protective Groups (2000).

Generally, the chemical transformations described throughout the specification may be carried out using substantially stoichiometric amounts of reactants, though certain reactions may benefit from using an excess of one or more of the reactants. Additionally, many of the reactions disclosed throughout the specification may be carried out at about room temperature (RT) and ambient pressure, but depending on reaction kinetics, yields, and so on, some reactions may be run at elevated pressures or employ higher temperatures (e.g., reflux conditions) or lower temperatures (e.g., −78 °C to 0 °C). Any reference in the disclosure and claims to a stoichiometric range, a temperature range, a pH range, etc., whether expressly using the word “range,” also includes the indicated endpoints.

Many of the chemical transformations may also employ one or more compatible solvents, which may influence the reaction rate and yield. Depending on the nature of the reactants, the one or more solvents may be polar protic solvents (including water), polar aprotic solvents, non-polar solvents, or some combination. Representative solvents include saturated aliphatic hydrocarbons (e.g., n-pentane, n-hexane, n-heptane, n-octane, cyclohexane, methylcyclohexane); aromatic hydrocarbons (e.g., benzene, toluene, xylenes); halogenated hydrocarbons (e.g., methylene chloride, chloroform, carbon tetrachloride); aliphatic alcohols (e.g., methanol, ethanol, propan-1-ol, propan-2-ol, butan-1-ol, 2-methyl-propan-1-ol, butan-2-ol, 2-methyl-propan-2-ol, pentan-1-ol, 3-methyl-butan-1-ol, hexan-1-ol, 2-methoxy-ethanol, 2-ethoxy-ethanol, 2-butoxy-ethanol, 2-(2-methoxy-ethoxy)-ethanol, 2-(2-ethoxy-ethoxy)-ethanol, 2-(2-butoxy-ethoxy)-ethanol); ethers (e.g., diethyl ether, di-isopropyl ether, dibutyl ether, 1,2-dimethoxy-ethane, 1,2-diethoxy-ethane, 1-methoxy-2-(2-methoxy-ethoxy)-ethane, 1-ethoxy-2-(2-ethoxy-ethoxy)-ethane, tetrahydrofuran, 1,4-dioxane); ketones (e.g., acetone, methyl ethyl ketone); esters (methyl acetate, ethyl acetate); nitrogen-containing solvents (e.g., formamide, N,N-dimethylformamide, acetonitrile, N-methyl-pyrrolidone, pyridine, quinoline, nitrobenzene); sulfur-containing solvents (e.g., carbon disulfide, dimethyl sulfoxide, tetrahydro-thiophene-1,1,-dioxide); and phosphorus-containing solvents (e.g., hexamethylphosphoric triamide).

In the schemes, below, substituent identifiers (e.g., X, Y, R¹, R², R³, R⁴, R⁵, R⁶, R⁷, and R⁸) are as defined above for Formula (I). As mentioned earlier, some of the starting materials and intermediates may include protecting groups, which are removed prior to the final product. In such cases, the substituent identifier refers to moieties defined in Formula (I) and to those moieties with appropriate protecting groups. For example, a starting material or intermediate in the synthetic methods may include a potentially reactive (secondary) amine. In such cases, the amine would include the moiety with or without, say, a Boc or Cbz group attached to the amine.

The production method of the compound of the present invention is explained below.

The raw material compound and reagent used and the compound obtained in each step in the following production method may be each in a form of a salt, and examples of such salt include those similar to the salts of the compound of the present invention and the like. When the compound obtained in each step is a free form, it can be converted to the objective salt according to a method known per se. When the compound obtained in each step is a salt, it can be converted to the objective free form or the other salt according to a method known per se.

The compound obtained in each step can be used directly as the reaction mixture or as a crude product for the next reaction. Alternatively, the compound obtained in each step can be isolated and purified from a reaction mixture according to a method known per se, for example, a separation means such as concentration, crystallization, recrystallization, distillation, solvent extraction, fractional distillation, column chromatography and the like.

When the raw material compound and reagent used in each step are commercially available, the commercially available product can also be used directly.

In the reaction in each step, while the reaction time varies depending on the kind of the reagent and solvent to be used, it is generally 1 min to 48 hr, preferably 10 min to 8 hr, unless otherwise specified.

In the reaction in each step, while the reaction temperature varies depending on the kind of the reagent and solvent to be used, it is generally −78 °C to 300 °C, preferably −78 °C to 150 °C, unless otherwise specified.

In the reaction in each step, while the pressure varies depending on the kind of the reagent and solvent to be used, it is generally 1 atm to 20 atm, preferably 1 atm to 3 atm, unless otherwise specified.

Microwave synthesizer such as Initiator manufactured by Biotage and the like may be used for the reaction in each step. While the reaction temperature varies depending on the kind of the reagent and solvent to be used, it is generally room temperature to 300 °C, preferably 50 °C to 250 °C, unless otherwise specified. While the reaction time varies depending on the kind of the reagent and solvent to be used, it is generally 1 min to 48 hr, preferably 1 min to 8 hr, unless otherwise specified.

In the reaction in each step, the reagent is used in an amount of 0.5 equivalents to 20 equivalents, preferably 0.8 equivalents to 5 equivalents, relative to the substrate, unless otherwise specified. When the reagent is used as a catalyst, the reagent is used in an amount of 0.001 equivalent to 1 equivalent, preferably 0.01 equivalent to 0.2 equivalent, relative to the substrate. When the reagent is used as a reaction solvent, the reagent is used in a solvent amount.

Unless otherwise specified, the reaction in each step is carried out without solvent, or by dissolving or suspending the raw material compound in a suitable solvent. Examples of the solvent include those described in Examples and the following solvents.
alcohols: methanol, ethanol, tert-butyl alcohol, 2-methoxyethanol and the like;
ethers: diethyl ether, diphenyl ether, tetrahydrofuran, 1,2-dimethoxyethane and the like;
aromatic hydrocarbons: chlorobenzene, toluene, xylene and the like;
saturated hydrocarbons: cyclohexane, hexane and the like;
amides: N,N-dimethylformamide, N-methylpyrrolidone and the like;
halogenated hydrocarbons: dichloromethane, carbon tetrachloride and the like;
nitriles: acetonitrile and the like;
sulfoxides: dimethyl sulfoxide and the like;
aromatic organic bases: pyridine and the like;
anhydrides: acetic anhydride and the like;
organic acids: formic acid, acetic acid, trifluoroacetic acid and the like;
inorganic acids: hydrochloric acid, sulfuric acid and the like;
esters: ethyl acetate and the like;
ketones: acetone, methyl ethyl ketone and the like;
water.
The above-mentioned solvent can be used in a mixture of two or more kinds thereof in an appropriate ratio.

When a base is used for the reaction in each step, examples thereof include those described in Examples and the following bases.
inorganic bases: sodium hydroxide, magnesium hydroxide, sodium carbonate, calcium carbonate, sodium hydrogen carbonate and the like;
organic bases: triethylamine, diethylamine, pyridine, 4-dimethylaminopyridine, N,N-dimethylaniline, 1,4-diazabicyclo[2.2.2]octane, 1,8-diazabicyclo[5.4.0]-7-undecene, imidazole, piperidine and the like;
metal alkoxides: sodium ethoxide, potassium tert-butoxide and the like;
alkali metal hydrides: sodium hydride and the like;
metal amides: sodium amide, lithium diisopropylamide, lithium hexamethyldisilazide and the like;
organic lithiums: n-butyllithium and the like.

When an acid or an acid catalyst is used for the reaction in each step, examples thereof include those described in Examples and the following acids and acid catalysts. inorganic acids: hydrochloric acid, sulfuric acid, nitric acid, hydrobromic acid, phosphoric acid and the like;
organic acids: acetic acid, trifluoroacetic acid, citric acid, p-toluenesulfonic acid, 10-camphorsulfonic acid and the like;
Lewis acid: boron trifluoride diethyl ether complex, zinc iodide, anhydrous aluminum chloride, anhydrous zinc chloride, anhydrous iron chloride and the like.

Unless otherwise specified, the reaction in each step is carried out according to a method known per se, for example, the method described in Jikken Kagaku Kouza, 5th Edition, vol. 13-19 (the Chemical Society of Japan ed.); Shin Jikken Kagaku Kouza, vol. 14-15 (the Chemical Society of Japan ed.); Fine Organic Chemistry, Revised 2nd Edition (L. F. Tietze, Th. Eicher, Nankodo); Organic Name Reactions, the Reaction Mechanism and Essence, Revised Edition (Hideo Togo, Kodansha); ORGANIC SYNTHESES Collective Volume I-VII (John Wiley & Sons Inc.); Modern Organic Synthesis in the Laboratory A Collection of Standard Experimental Procedures (Jie Jack Li, OXFORD UNIVERSITY); Comprehensive Heterocyclic Chemistry III, Vol. 1-Vol. 14 (Elsevier Japan); Strategic Applications of Named Reactions in Organic Synthesis (translated by Kiyoshi Tomioka, Kagakudojin); Comprehensive Organic Transformations (VCH Publishers Inc.), 1989, or the like, or the method described in Examples.

The methods depicted in the schemes may be varied as desired. For example, protecting groups may be added or removed and products may be further elaborated via, for example, alkylation, acylation, hydrolysis, oxidation, reduction, amidation, sulfonation, alkynylation, and the like to give the desired final product. Furthermore, any intermediate or final product which comprises mixture of stereoisomers may be optionally purified by chiral column chromatography (e.g., supercritical fluid chromatography) or by derivatization with optically-pure reagents as described above to give a desired stereoisomer.

In each step, the protection or deprotection reaction of a functional group is carried out according to a method known per se, for example, the method described in “Protective Groups in Organic Synthesis, 4th Ed”, Wiley-Interscience, Inc., 2007 (Theodora W. Greene, Peter G. M. Wuts); “Protecting Groups 3rd Ed.” Thieme, 2004 (P. J. Kocienski), or the like, or the method described in Examples.

Examples of the protecting group for a hydroxy group of an alcohol and the like and a phenolic hydroxy group include ether-type protecting groups such as methoxymethyl ether, benzyl ether, tert-butyldimethylsilyl ether, tetrahydropyranyl ether and the like; carboxylate ester-type protecting groups such as acetate ester and the like; sulfonate ester-type protecting groups such as methanesulfonate ester and the like; carbonate ester-type protecting groups such as tert-butylcarbonate and the like, and the like.

Examples of the protecting group for a carbonyl group of an aldehyde include acetal-type protecting groups such as dimethylacetal and the like; cyclic acetal-type protecting groups such as 1,3-dioxane and the like, and the like.

Examples of the protecting group for a carbonyl group of a ketone include ketal-type protecting groups such as dimethylketal and the like; cyclic ketal-type protecting groups such as 1,3-dioxane and the like; oxime-type protecting groups such as O-methyloxime and the like; hydrazone-type protecting groups such as N,N-dimethylhydrazone and the like, and the like.

Examples of the protecting group for a carboxyl group include ester-type protecting groups such as methyl ester and the like; amide-type protecting groups such as N,N-dimethylamide and the like, and the like.

Examples of the protecting group for a thiol include ether-type protecting groups such as benzyl thioether and the like; ester-type protecting groups such as thioacetate ester, thiocarbonate, thiocarbamate and the like, and the like.

Examples of the protecting group for an amino group and an aromatic heterocycle such as imidazole, pyrrole, indole and the like include carbamate-type protecting groups such as benzyl carbamate and the like; amide-type protecting groups such as acetamide and the like; alkyl amine-type protecting groups such as N-triphenylmethylamine and the like; sulfonamide-type protecting groups such as methanesulfonamide and the like, and the like.

The protecting groups can be removed according to a method known per se, for example, by employing a method using acid, base, ultraviolet rays, hydrazine, phenylhydrazine, sodium N-methyldithiocarbamate, tetrabutylammonium fluoride, palladium acetate, trialkylsilyl halide (e.g., trimethylsilyl iodide, trimethylsilyl bromide) and the like, a reduction method, and the like.

When reduction reaction is carried out in each step, examples of the reducing agent to be used include metal hydrides such as lithium aluminum hydride, sodium triacetoxyborohydride, sodium cyanoborohydride, diisobutylaluminum hydride (DIBAL-H), sodium borohydride, tetramethylammonium triacetoxyborohydride and the like; boranes such as borane tetrahydrofuran complex and the like; Raney nickel; Raney cobalt; hydrogen; formic acid; triethylsilane; iron; zinc and the like. When carbon-carbon double bond or triple bond is reduced, a method using a catalyst such as palladium-carbon, Lindlar's catalyst and the like may be employed.

When oxidation reaction is carried out in each step, examples of the oxidizing agent to be used include peroxides such as m-chloroperbenzoic acid (mCPBA), hydrogen peroxide, tert-butylhydroperoxide and the like; perchlorates such as tetrabutylammonium perchlorate and the like; chlorates such as sodium chlorate and the like; chlorites such as sodium chlorite and the like; periodates such as sodium periodate and the like; hypervalent iodine reagents such as iodosylbenzene and the like; reagents containing manganese such as manganese dioxide, potassium permanganate and the like; leads such as lead tetraacetate and the like; reagents containing chromium such as pyridinium chlorochromate (PCC), pyridinium dichromate (PDC), Jones reagent and the like; halogen compounds such as N-bromosuccinimide (NBS) and the like; oxygen; ozone; sulfur trioxide-pyridine complex; osmium tetroxide; selenium dioxide; 2,3-dichloro-5,6-dicyano-1,4-benzoquinone (DDQ) and the like.

When radical reaction is carried out in each step, examples of the radical initiator to be used include azo compounds such as azobisisobutyronitrile (AIBN) and the like; water-soluble radical initiators such as 4,4′-azobis-4-cyanopentanoic acid (ACPA) and the like; triethylboron in the presence of air or oxygen; benzoyl peroxide and the like. Examples of the radical reagent to be used include tributylstannane, tristrimethylsilylsilane, 1,1,2,2-tetraphenyldisilane, diphenylsilane, samarium iodide and the like.

When Wittig reaction is carried out in each step, examples of the Wittig reagent to be used include alkylidene phosphoranes and the like. The alkylidene phosphoranes can be prepared according to a method known per se, for example, by reacting a phosphonium salt with a strong base.

When Horner-Emmons reaction is carried out in each step, examples of the reagent to be used include phosphonoacetates such as methyl dimethylphosphonoacetate, ethyl diethylphosphonoacetate and the like; and bases such as alkali metal hydrides, organic lithiums and the like.

When Friedel-Crafts reaction is carried out in each step, a combination of a Lewis acid and an acid chloride or a combination of a Lewis acid and an alkylating agent (e.g., an alkyl halide, an alcohol, an olefin etc.) is used as a reagent. Alternatively, an organic acid or an inorganic acid can also be used instead of a Lewis acid, and an anhydride such as acetic anhydride and the like can also be used instead of an acid chloride.

When aromatic nucleophilic substitution reaction is carried out in each step, a nucleophile (e.g., an amine, imidazole etc.) and a base (e.g., an inorganic base, an organic base etc.) are used as a reagent.

When nucleophilic addition reaction by a carbo anion, nucleophilic 1,4-addition reaction (Michael addition reaction) by a carbo anion or nucleophilic substitution reaction by a carbo anion is carried out in each step, and examples of the base to be used for generation of the carbo anion include organic lithiums, metal alkoxides, inorganic bases, organic bases and the like.

When Grignard reaction is carried out in each step, examples of the Grignard reagent to be used include arylmagnesium halides such as phenylmagnesium bromide and the like; and alkylmagnesium halides such as methylmagnesium bromide and the like. The Grignard reagent can be prepared according to a method known per se, for example, by reacting an alkyl halide or an aryl halide with a metal magnesium in an ether or tetrahydrofuran as a solvent.

When Knoevenagel condensation reaction is carried out in each step, a compound having an activated methylene group with two electron withdrawing groups (e.g., malonic acid, diethyl malonate, malononitrile etc.) and a base (e.g., an organic base, a metal alkoxide, an inorganic base) are used as a reagent.

When Vilsmeier-Haack reaction is carried out in each step, phosphoryl chloride and an amide derivative (e.g., N,N-dimethylformamide etc.) are used as a reagent.

When azidation reaction of an alcohol, an alkyl halide or a sulfonate is carried out in each step, examples of the azidating agent to be used include diphenylphosphorylazide (DPPA), trimethylsilylazide, sodium azide and the like. For example, for the azidation reaction of an alcohol, a method using diphenylphosphorylazide and 1,8-diazabicyclo[5.4.0]undec-7-ene (DBU), a method using trimethylsilylazide and a Lewis acid, and the like are employed.

When reductive amination reaction or reductive alkylation reaction is carried out in each step, examples of the reducing agent to be used include sodium triacetoxyborohydride, sodium cyanoborohydride, hydrogen, formic acid and the like. When the substrate is an amine compound, examples of the carbonyl compound to be used include paraformaldehyde, aldehydes such as acetaldehyde and the like, and ketones such as cyclohexanone and the like. When the substrate is a carbonyl compound, examples of the amine to be used include ammonia, primary amines such as methylamine and the like; secondary amines such as dimethylamine and the like, and the like.

When Mitsunobu reaction is carried out in each step, a cyanomethylenetrialkyl phosphorane (e.g., cyanomethylenetrimethylphosphorane, cyanomethylenetributylphosphorane), or a combination of an azodicarboxylate (e.g., diethyl azodicarboxylate (DEAD), diisopropyl azodicarboxylate (DIAD) etc.) and a phosphine (e.g., triphenylphosphine, tri-n-butylphosphine) is used as a reagent.

When esterification reaction, or amidation reaction or urea formation reaction is carried out in each step, examples of the reagent to be used include acyl halides such as acid chlorides, acid bromides and the like; activated carboxylic acids such as anhydrides, activated esters, sulfates and the like. Examples of the activating agent of the carboxylic acid include carbodiimide condensing agents such as 1-ethyl-3-(3-dimethylaminopropyl)carbodiimide hydrochloride (WSCD) and the like; triazine condensing agents such as 4-(4,6-dimethoxy-1,3,5-triazin-2-yl)-4-methylmorpholinium chloride n-hydrate (DMT-MM) and the like; carbonate condensing agents such as 1,1-carbonyldiimidazole (CDI) and the like; diphenylphosphoryl azide (DPPA); benzotriazol-1-yloxy-trisdimethylaminophosphonium salt (BOP reagent); 2-chloro-1-methyl-pyridinium iodide (Mukaiyama reagent); thionyl chloride; lower alkyl haloformates such as ethyl chloroformate and the like; O-(7-azabenzotriazol-1-yl)-N,N,N′,N′-tetramethyluronium hexafluorophosphorate (HATU); sulfuric acid; combinations thereof and the like. When carbodiimide condensing agent is used, an additive such as 1-hydroxybenzotriazole (HOBt), N-hydroxysuccinimide (HOSu), dimethylaminopyridine (DMAP) and the like may be added to the reaction system.

When carbamylation reaction is carried out in each step, examples of the reagent to be used include alkyl chloroformates such as methyl chloroformate and the like. Examples of the base include organic bases, sodium carbonate and the like.

When carbamoylation reaction is carried out in each step, examples of the activating agents to be used include triphosgene, 1,1-carbonyldiimidazole, 4-nitrophenyl chloroformate and the like, examples of the base include organic bases, sodium carbonate and the like, and examples of the amine include ammonia, primary amines such as methylamine and the like; secondary amines such as dimethylamine and the like, and the like.

When guanidine formation reaction is carried out in each step, examples of the activating agent include 1-(3-dimethylaminopropyl)-3-ethylcarbodiimide hydrochloride (WSCI) and the like. Examples of the base include organic bases and the like.

When coupling reaction is carried out in each step, examples of the metal catalyst to be used include palladium compounds such as palladium(II) acetate, tetrakis(triphenylphosphine)palladium(0), dichlorobis(triphenylphosphine)palladium(II), dichlorobis(triethylphosphine)palladium(II), tris(dibenzylideneacetone)dipalladium(0), 1,1′-bis(diphenylphosphino)ferrocenepalladium(II) chloride and the like; nickel compounds such as tetrakis(triphenylphosphine)nickel(0) and the like; rhodium compounds such as tris(triphenylphosphine)rhodium(III) chloride and the like; cobalt compounds; copper compounds such as copper oxide, copper(I) iodide and the like; platinum compounds and the like. In addition, a base can be added to the reaction system, and examples thereof include inorganic bases, metal alkoxides and the like.

When thiocarbonylation reaction is carried out in each step, phosphorus pentasulfide is typically used as the thiocarbonylating agent. Alternatively, a reagent having a 1,3,2,4-dithiadiphosphetane-2,4-disulfide structure (e.g., 2,4-bis(4-methoxyphenyl)-1,3,2,4-dithiadiphosphetane-2,4-disulfide (Lawesson's reagent) etc.) can also be used instead of phosphorus pentasulfide.

When Wohl-Ziegler reaction is carried out in each step, examples of the halogenating agent to be used include N-iodosuccinimide, N-bromosuccinimide (NBS), N-chlorosuccinimide (NCS), bromine, sulfuryl chloride and the like. In addition, the reaction can be accelerated by subjecting a radical initiator such as heat, light, benzoyl peroxide, azobisisobutyronitrile and the like to the reaction system.

When halogenation reaction of a hydroxy group is carried out in each step, examples of the halogenating agent to be used include hydrohalic acids and acid halides of inorganic acids, specifically, hydrochloric acid, thionyl chloride, phosphorus oxychloride and the like for chlorination, 48% hydrobromic acid and the like for bromination. In addition, a method of producing an alkyl halide by reacting an alcohol with triphenylphosphine and carbon tetrachloride or carbon tetrabromide or the like can be employed. Alternatively, a method of producing an alkyl halide via two steps comprising converting an alcohol to the corresponding sulfonate, and then reacting the sulfonate with lithium bromide, lithium chloride or sodium iodide can also be employed.

When Arbuzov reaction is carried out in each step, examples of the reagent to be used include alkyl halides such as ethyl bromoacetate and the like; and phosphites such as triethyl phosphite, tri(isopropyl) phosphite and the like.

When sulfonate esterification reaction is carried out in each step, examples of the sulfonating agent to be used include methanesulfonyl chloride, p-toluenesulfonyl chloride, methanesulfonic anhydride, p-toluenesulfonic anhydride and the like.

When hydrolysis reaction is carried out in each step, an acid or a base is used as a reagent. For acid hydrolysis reaction of tert-butyl ester, formic acid, triethylsilane and the like may be added to reductively-trap tert-butyl cation which is by-produced.

When dehydration reaction is carried out in each step, examples of the dehydrating agent to be used include sulfuric acid, diphosphorus pentaoxide, phosphorus oxychloride, N,N′-dicyclohexylcarbodiimide, alumina, polyphosphoric acid and the like.

When Chan-Lam reaction is carried out in each step, examples of the metal catalyst to be used include copper compounds such as copper(I) bromide, copper(I) iodide, copper(II) acetate and the like. In addition, a base may be added to the reaction system, and examples thereof include organic bases and the like.

When Ullmann reaction is carried out in each step, examples of the metal catalyst to be used include copper compounds such as copper(I) bromide, copper(I) iodide, copper(II) acetate and the like, and examples of the ligand include N,N,N′,N′-tetramethylethylene diamine and the like. In addition, a base may be added to the reaction system, and examples thereof include organic bases, inorganic bases and the like.

When alkylation reaction is carried out in each step, examples of the base to be used include potassium carbonate, tripotassium phosphate, triethylamine, N,N-diisopropylethylamine, pyridine, sodium ethoxide, potassium tert-butoxide, sodium hydride, lithiumhexamethyldisilazide, sodium hexamethyldisilazide, n-butyllithium and the like.

When deoxofluorination reaction is carried out in each step, examples of the fluorinating agent to be used include bis(2-methoxyethyl)aminosulfur trifluoride, diethylaminosulfur trifluoride, 4-tert-butyl-2,6-dimethylphenylsulfur trifluoride, N,N-diethyl-S,S-difluorosulfiliminium tetrafluoroborate, difluoro-4-morpholinylsulfonium tetrafluoroborate and the like.

When Hofmann rearrangement reaction is carried out in each step, examples of the reagent to be used include lead tetraacetate, iodobenzene diacetate and the like.

When thiourea formation reaction or thiocarbamation reaction is carried out in each step, examples of the reagent to be used include thiophosgene and the like, and examples of the base include organic bases, sodium hydride and the like.

When cyclopropanation reaction is carried out in each step, examples of the reagent to be used include diiodomethane, dibromomethane, dibromodifluoromethane, dibromofluoromethane, (trifluoromethyl)trimethylsilane, (bromodifluoromethyl)trimethylsilane, (dibromofluoromethyl)trimethylsilane, carbon tetrafluoride, sodium trifluoroacetate and the like.

When Compound (I) and intermediate for the production of Compound (I) have a convertible functional group (e.g., a carboxyl group, an amino group, a hydroxy group, a carbonyl group, a mercapto group, a C₁₋₆ alkoxy-carbonyl group, a C₆₋₁₄ aryloxy-carbonyl group, a C₇₋₁₆ aralkyloxy-carbonyl group, a sulfo group, a halogen atom, an optionally halogenated C₁₋₆ alkylsulfonyloxy group, a cyano group, an aminocarbonyl group, a boryl group etc.), various compounds can be produced by converting such functional group according to a method known per se or a method analogous thereto.

Carboxyl group can be converted, for example, by reactions such as esterification, reduction, amidation, conversion reaction to optionally protected amino group and the like.

Amino group can be converted, for example, by reactions such as amidation, sulfonylation, nitrosation, alkylation, arylation, imidation and the like.

Hydroxy group can be converted, for example, by reactions such as esterification, carbamoylation, sulfonylation, alkylation, fluorination, arylation, oxidation, halogenation and the like.

Carbonyl group can be converted, for example, by reactions such as reduction, oxidation, fluorination, imination (including oximation, hydrazonation), (thio)ketalization, alkylidenation, thiocarbonylation and the like.

Mercapto group can be converted, for example, by reactions such as alkylation, oxidation and the like.

C₁₋₆ alkoxy-carbonyl group, C₆₋₁₄ aryloxy-carbonyl group and C₇₋₁₆ aralkyloxy-carbonyl group can be converted, for example, by reactions such as reduction, hydrolysis and the like.

Sulfo group can be converted, for example, by reactions such as sulfonamidation, reduction and the like.

Halogen atom can be converted, for example, by various nucleophilic substitution reactions, various coupling reactions and the like.

Optionally halogenated C₁₋₆ alkylsulfonyloxy group can be converted, for example, by various nucleophilic substitution reactions, various coupling reactions and the like.

Cyano group can be converted, for example, by reactions such as reduction, hydrolysis and the like.

Aminocarbonyl group can be converted, for example, by reactions such as dehydration, reduction and the like.

Boryl group can be converted, for example, by oxidation, various coupling reactions and the like.

In each of the above-mentioned reactions, when the compound is obtained in a free form, it may be converted to a salt according to a conventional method. When it is obtained as a salt, it may be converted to a free form or other salt according to a conventional method.

The conversion of these functional group can be carried out according to a method known per se, for example, the method described in Comprehensive Organic Transformations, Second Edition, Wiley-VCH, Richard C. Larock, or the like.

Compound (I) obtained in each reaction scheme can be isolated and purified by known separation and purification means such as concentration, concentration under reduced pressure, solvent extraction, crystallization, recrystallization, phase transfer, chromatography and the like. In addition, each material compound used in each reaction scheme can be isolated and purified by those similar to the above-mentioned known separation and purification means. The material compound may be used directly in the next step as the reaction mixture without isolation.

When Compound (I) has isomers such as an optical isomer, a stereoisomer, a regioisomer and a rotamer and the like, such isomers and a mixture thereof are also encompassed in Compound (I). For example, when Compound (I) has an optical isomer, the optical isomer resolved from racemate is also encompassed in Compound (I). These isomers can be obtained as single products according to synthetic methods known per se, separation methods known per se (e.g., concentration, solvent extraction, column chromatography, recrystallization etc.), optical resolutions (e.g., fractional recrystallization method, chiral column method, diastereomer method and the like).

Compound (I) may be a crystal, and the crystal form may be single or a mixture of crystal forms, both of which are encompassed in compound (I). The crystal can be produced according to a crystallization method known per se.

Compound (I) may be a solvate (e.g., hydrate) or a non-solvate (e.g., non-hydrate etc.) and both are encompassed in compound (I).

The compounds labeled with isotopes (e.g., ³H, ¹¹C, ¹⁴C, ¹⁸F, ³⁵S, ¹²⁵I etc.) and the like are also encompassed in Compound (I).

A deuterium conversion form wherein ¹H is converted to ²H(D) is also encompassed in Compound (I).

Compound (I) labeled or substituted with an isotope can be used as, for example, a tracer (PET tracer) used for Positron Emission Tomography (PET), and therefore, it is useful in the fields of medical diagnosis and the like.

Compound (I) of the present invention can be synthesized according to the production method described below.

Each variable in the formulas of the reaction schemes is as defined above, unless otherwise specified.

Compound (I) can be produced from compound (1) according to the following scheme (Scheme I). In the scheme, P¹ is a protecting group for phenolic hydroxyl group, A is a halogen atom, and Z¹ is a dihydroxyboryl group, a pinacolboryl group (i.e., 4,4,5,5-tetramethyl-1,3,2-dioxaborolan-2-yl (Bpin)) or a halogen atom. Examples of the protecting group for phenolic hydroxyl group include a methoxymethyl group, a benzyl group, a 4-methoxbenzyl group and the like.

Compound (2) can be produced by subjecting compound (1) to a halogenation reaction.

Compound (3) can be produced by subjecting compound (2) to Chan-Lam coupling with the corresponding aryl boronate ester or boronic acid. Alternatively, compound (3) can also be produced by subjecting compound (2) to Ullmann reaction, or an aromatic nucleophilic substitution reaction with the corresponding aryl halide.

Compound (4) can be produced by subjecting compound (3) to a reduction reaction.

Compound (5) can be produced by subjecting compound (4) to a thiourea formation with R¹R²NH.

Compound (6) wherein X is S can be produced by subjecting compound (5) to Ullmann reaction. Alternatively, compound (6) wherein X is NR⁷ can be produced by subjecting compound (7) to Ullmann reaction.

Compound (7) can be produced by subjecting compound (5) to a guanidine formation reaction with R⁷—NH₂.

Compound (I) can be produced by subjecting compound (6) to a deprotection reaction.

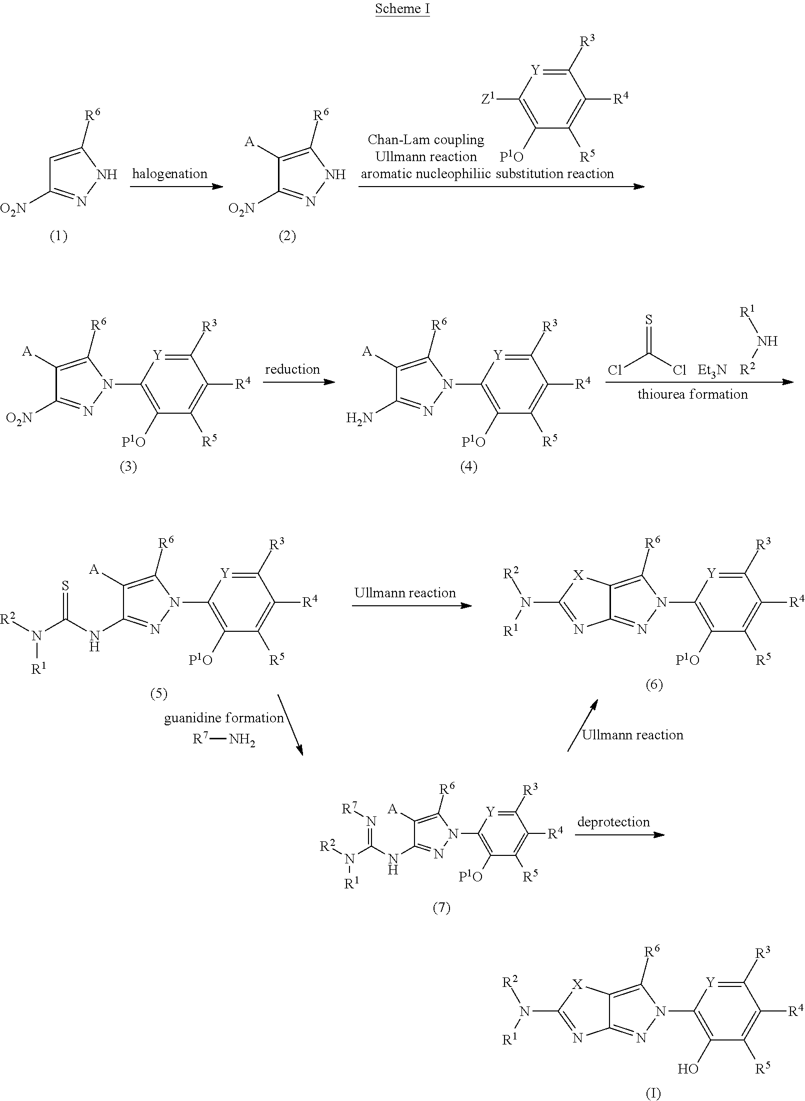

Compound (I-a), an embodiment of Compound (I) wherein R¹ is optionally substituted 5- or 6-membered nitrogen-containing non-aromatic heterocyclic group, can be produced from compound (6′) according to the following scheme (Scheme II). In the scheme, P² is a protecting group for amine. Examples of the protecting group for amine include a Boc group, a Cbz group, a Troc group, a PMB group, a Bn group and the like. R¹¹ is preferably an alkyl group, or an acyl group (e.g., a C₁₋₆ alkyl-carbonyl group, a C₁₋₆ alkoxy-carbonyl group, a C₁₋₆ alkyl-carbamoyl group, a C₁₋₆ sulfonyl group and the like).

Compound (8) can be produced by subjecting compound (6′) to a deprotection reaction.

Compound (9) can be produced by subjecting compound (8) to an N-functionalization reaction, including a reductive amination reaction, an alkylation reaction, or an acylation reaction, a carbamoylation reaction, a carbamylation reaction, or, a sulfonylation reaction and the like.

Compound (I-a) can be produced by subjecting compound (9) to a deprotection reaction.

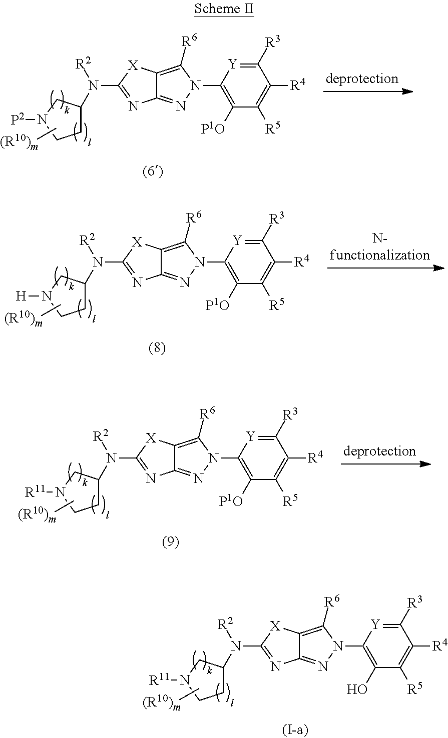

Compound (5) in the above-mentioned Scheme I can be also produced from compound (1) according to the following scheme (Scheme III). In the scheme, P¹, A and Z¹ are as defined above, and Y′ is C—W or N, wherein W is a halogen atom.

Compound (10) can be produced by subjecting compound (1) to Chan-Lam coupling with the corresponding aryl boronate ester or boronic acid. Alternatively, compound (10) can also be produced by subjecting compound (1) to Ullmann reaction, or an aromatic nucleophilic substitution reaction with the corresponding aryl halide.

Compound (10′) can be produced by subjecting compound (10) wherein Y′ is C—W to a Suzuki coupling reaction with R⁸-Bpin, R⁸—B(OH)₂, or R⁸—BF₃K.

Compound (11) can be produced by subjecting compound (10), wherein Y′ is N, to a reduction reaction. Compound (11) can also be produced by subjecting compound (10′) to a reduction reaction.

Compound (4) can be produced by subjecting compound (11) to a halogenation reaction.

Compound (5) can be produced by subjecting compound (4) to a thiourea formation with R¹R²NH.

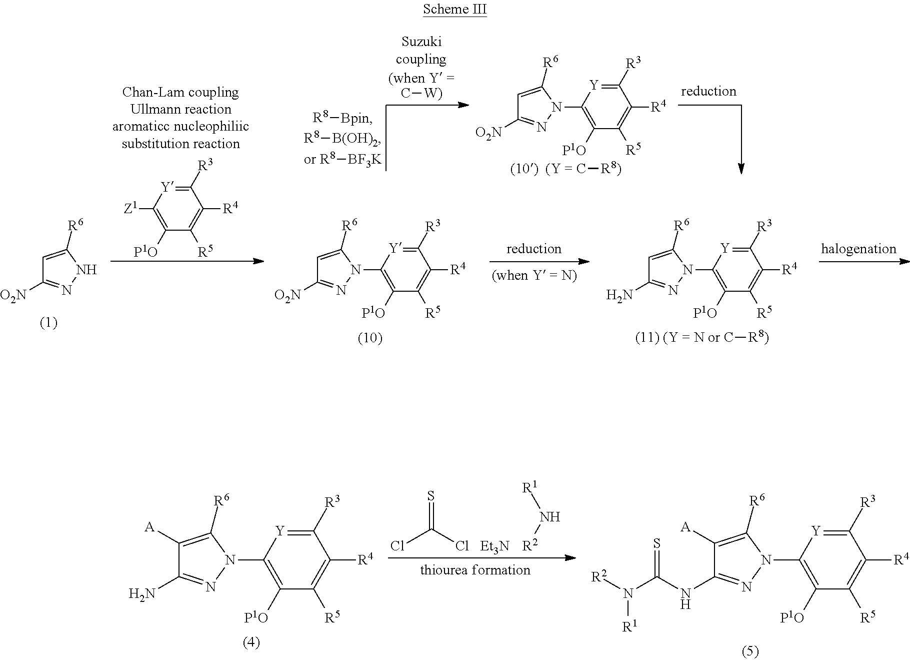

For production of Compound (I), wherein Y is CR⁸ and R⁸ is optionally halogenated cyclopropyl, the corresponding compound (11) can be used, which can be produced from compound (10), wherein Y′ is C—W, according to the following scheme (Scheme IV). In the scheme, B¹ and B² are each independently selected from a hydrogen and a halogen atom, and P¹ and W are as defined above. B¹ and B² are supplied by a regent used in cyclopropanation as mentioned above.

Compound (10′a) can be produced by subjecting compound (10), wherein Y′ is C—W, to a Suzuki coupling reaction with CH₂═CH—BF₃K.

Compound (10″a) can be produced by subjecting compound (10′a) to a cyclopropanation reaction.

Compound (11), wherein Y is CR⁸ and R⁸ is optionally halogenated cyclopropyl, can be produced by subjecting compound (10″a) to a reduction reaction.

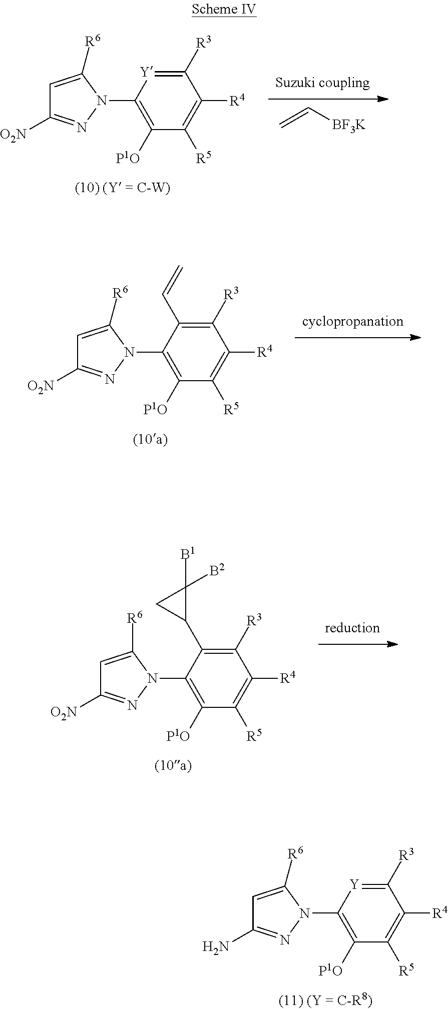

For production of Compound (I), wherein Y is CR⁸ and R⁸ is an isopropyl, the corresponding compound (11) can be used, which can be produced from compound (10), wherein Y′ is C—W, according to the following scheme (Scheme V). In the scheme, P¹ and W are as defined above.

Compound (10′b) can be produced by subjecting compound (10), wherein Y′ is C—W, to a Suzuki coupling reaction with CH₂═C(Me)Bpin.

Compound (11), wherein Y is CR⁸ and R⁸ is an isopropyl, can be produced by subjecting compound (10′b) to a reduction reaction.

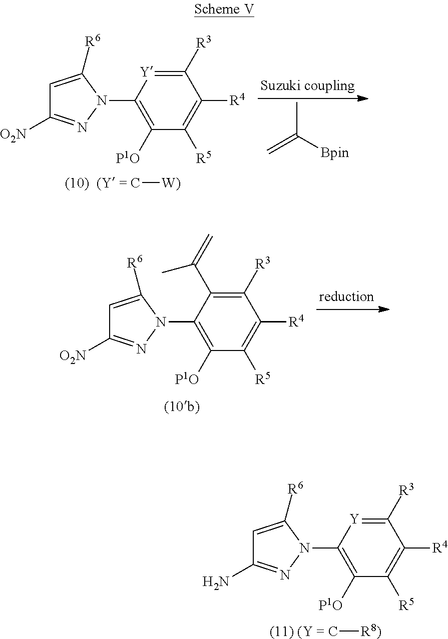

Compounds of Formula (I), which include compounds named above, and their pharmaceutically acceptable complexes, salts, solvates and hydrates, should be assessed for their biopharmaceutical properties, such as solubility and solution stability across pH, permeability, and the like, to select an appropriate dosage form and route of administration. Compounds that are intended for pharmaceutical use may be administered as crystalline or amorphous products, and may be obtained, for example, as solid plugs, powders, or films by methods such as precipitation, crystallization, freeze drying, spray drying, evaporative drying, microwave drying, or radio frequency drying.

Compound (I) may be administered alone or in combination with one another or with one or more pharmacologically active agents which are different than Compound (I). When administering Compound (I) with a pharmacologically active agent (“concomitant drug”), the administration time of Compound (I) and the concomitant drug is not restricted, and Compound (I) or a pharmaceutical composition thereof, or the concomitant drug or a pharmaceutical composition thereof can be administered to a subject simultaneously, or may be administered at different times. The dosage of the concomitant drug may be determined according to the dose clinically used, and can be appropriately selected depending on a subject, administration route, disease, combination and the like.

The administration mode of the combination of Compound (I) and the concomitant drug is not particularly limited, and Compound (I) and the concomitant drug only need to be combined on administration. Examples of such administration mode include the following: (1) administration of a single preparation obtained by simultaneously processing Compound (I) and the concomitant drug, (2) simultaneous administration of two kinds of preparations of Compound (I) and the concomitant drug, which have been separately produced, by the same administration route, (3) administration of two kinds of preparations of Compound (I) and the concomitant drug, which have been separately produced, by the same administration route in a staggered manner, (4) simultaneous administration of two kinds of preparations of Compound (I) and the concomitant drug, which have been separately produced, by different administration routes, (5) administration of two kinds of preparations of Compound (I) and the concomitant drug, which have been separately produced, by different administration routes in a staggered manner (e.g., administration in the order of Compound (I) and the concomitant drug, or in the reverse order) and the like.

The dose of the concomitant drug can be appropriately determined based on the dose employed in clinical situations. The mixing ratio of Compound (I) and a concomitant drug can be appropriately determined depending on the administration subject, administration route, target disease, symptom, combination and the like.

For example, the content of Compound (I) in the combination with a concomitant drug differs depending on the form of a preparation, and usually from about 0.01 to about 100 wt %, preferably from about 0.1 to about 50 wt %, further preferably from about 0.5 to about 20 wt %, based on the whole preparation.

The content of the concomitant drug used in the combination with Compound (I) differs depending on the form of a preparation, and usually from about 0.01 to about 100 wt %, preferably from about 0.1 to about 50 wt %, further preferably from about 0.5 to about 20 wt %, based on the whole preparation.

The content of additives such as a carrier and the like used in the combination of Compound (I) and a concomitant drug differs depending on the form of a preparation, and usually from about 1 to about 99.99 wt %, preferably from about 10 to about 90 wt %, based on the preparation.

Similar contents may be employed even when Compound (I) and a concomitant drug are separately formulated into preparations.

Generally, one or more of these compounds are administered as a pharmaceutical composition (a formulation) in association with one or more pharmaceutically acceptable excipients. The choice of excipients depends on the mode of administration, the effect of the excipient on solubility and stability, and the nature of the dosage form, among other things. Useful pharmaceutical compositions and methods for their preparation may be found, for example, in A. R. Gennaro (ed.), Remington: The Science and Practice of Pharmacy (20th ed., 2000).

Compound (I) may be administered orally. Oral administration may involve swallowing in which case the compound enters the bloodstream via the gastrointestinal tract. Alternatively, or additionally, oral administration may involve mucosal administration (e.g., buccal, sublingual, supralingual administration) such that the compound enters the bloodstream through the oral mucosa.

Formulations suitable for oral administration include solid, semi-solid and liquid systems such as tablets; soft or hard capsules containing multi- or nano-particulates, liquids, or powders; lozenges which may be liquid-filled; chews; gels; fast dispersing dosage forms; films; ovules; sprays; and buccal or mucoadhesive patches. Liquid formulations include suspensions, solutions, syrups and elixirs. Such formulations may be employed as fillers in soft or hard capsules (made, e.g., from gelatin or hydroxypropylmethylcellulose) and typically comprise a carrier (e.g., water, ethanol, polyethylene glycol, propylene glycol, methylcellulose, or a suitable oil) and one or more emulsifying agents, suspending agents or both. Liquid formulations may also be prepared by the reconstitution of a solid (e.g., from a sachet).

Compound (I) may also be used in fast-dissolving, fast-disintegrating dosage forms such as those described in Liang and Chen, Expert Opinion in Therapeutic Patents (2001) 11(6):981-986.

For tablet dosage forms, depending on dose, the active pharmaceutical ingredient (API) may comprise from about 1 wt % to about 80 wt % of the dosage form or more typically from about 5 wt % to about 60 wt % of the dosage form. In addition to the API, tablets may include one or more disintegrants, binders, diluents, surfactants, glidants, lubricants, anti-oxidants, colorants, flavoring agents, preservatives, and taste-masking agents. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, C₁₋₆ alkyl-substituted hydroxypropylcellulose, starch, pregelatinized starch, and sodium alginate. Generally, the disintegrant will comprise from about 1 wt % to about 25 wt % or from about 5 wt % to about 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropylcellulose and hydroxypropylmethylcellulose. Tablets may also contain diluents, such as lactose (monohydrate, spray-dried monohydrate, anhydrous), mannitol, xylitol, dextrose, sucrose, sorbitol, microcrystalline cellulose, starch and dibasic calcium phosphate dihydrate.

Tablets may also include surface active agents, such as sodium lauryl sulfate and polysorbate 80, and glidants such as silicon dioxide and talc. When present, surface active agents may comprise from about 0.2 wt % to about 5 wt % of the tablet, and glidants may comprise from about 0.2 wt % to about 1 wt % of the tablet.

Tablets may also contain lubricants such as magnesium stearate, calcium stearate, zinc stearate, sodium stearyl fumarate, and mixtures of magnesium stearate with sodium lauryl sulfate. Lubricants may comprise from about 0.25 wt % to about 10 wt % or from about 0.5 wt % to about 3 wt % of the tablet.

Tablet blends may be compressed directly or by roller compaction to form tablets. Tablet blends or portions of blends may alternatively be wet-, dry-, or melt-granulated, melt congealed, or extruded before tableting. If desired, prior to blending one or more of the components may be sized by screening or milling or both. The final dosage form may comprise one or more layers and may be coated, uncoated, or encapsulated. Exemplary tablets may contain up to about 80 wt % of API, from about 10 wt % to about 90 wt % of binder, from about 0 wt % to about 85 wt % of diluent, from about 2 wt % to about 10 wt % of disintegrant, and from about 0.25 wt % to about 10 wt % of lubricant. For a discussion of blending, granulation, milling, screening, tableting, coating, as well as a description of alternative techniques for preparing drug products, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997).

Consumable oral films for human or veterinary use are pliable water-soluble or water-swellable thin film dosage forms which may be rapidly dissolving or mucoadhesive. In addition to the API, a typical film includes one or more film-forming polymers, binders, solvents, humectants, plasticizers, stabilizers or emulsifiers, viscosity-modifying agents, and solvents. Other film ingredients may include anti-oxidants, colorants, flavorants and flavor enhancers, preservatives, salivary stimulating agents, cooling agents, co-solvents (including oils), emollients, bulking agents, anti-foaming agents, surfactants, and taste-masking agents. Some components of the formulation may perform more than one function.

In addition to dosing requirements, the amount of API in the film may depend on its solubility. If water soluble, the API would typically comprise from about 1 wt % to about 80 wt % of the non-solvent components (solutes) in the film or from about 20 wt % to about 50 wt % of the solutes in the film. A less soluble API may comprise a greater proportion of the composition, typically up to about 88 wt % of the non-solvent components in the film.

The film-forming polymer may be selected from natural polysaccharides, proteins, or synthetic hydrocolloids and typically comprises from about 0.01 wt % to about 99 wt % or from about 30 wt % to about 80 wt % of the film.

Film dosage forms are typically prepared by evaporative drying of thin aqueous films coated onto a peelable backing support or paper, which may be carried out in a drying oven or tunnel (e.g., in a combined coating-drying apparatus), in lyophilization equipment, or in a vacuum oven.

Useful solid formulations for oral administration may include immediate release formulations and modified release formulations. Modified release formulations include delayed-, sustained-, pulsed-, controlled-, targeted-, and programmed-release. For a general description of suitable modified release formulations, see U.S. Pat. No. 6,106,864. For details of other useful release technologies, such as high energy dispersions and osmotic and coated particles, see Verma et al, Pharmaceutical Technology On-line (2001) 25(2):1-14.

Compound (I) may also be administered directly into the blood stream, muscle, or an internal organ of the subject. Suitable techniques for parenteral administration include intravenous, intraarterial, intraperitoneal, intrathecal, intraventricular, intraurethral, intrasternal, intracranial, intramuscular, intrasynovial, and subcutaneous administration. Suitable devices for parenteral administration include needle injectors, including microneedle injectors, needle-free injectors, and infusion devices.

Parenteral formulations are typically aqueous solutions which may contain excipients such as salts, carbohydrates and buffering agents (e.g., pH of from about 3 to about 9). For some applications, however, Compound (I) may be more suitably formulated as a sterile non-aqueous solution or as a dried form to be used in conjunction with a suitable vehicle such as sterile, pyrogen-free water. The preparation of parenteral formulations under sterile conditions (e.g., by lyophilization) may be readily accomplished using standard pharmaceutical techniques.

The solubility of compounds which are used in the preparation of parenteral solutions may be increased through appropriate formulation techniques, such as the incorporation of solubility-enhancing agents. Formulations for parenteral administration may be formulated to be immediate or modified release. Modified release formulations include delayed, sustained, pulsed, controlled, targeted, and programmed release. Thus, Compounds (I) may be formulated as a suspension, a solid, a semi-solid, or a thixotropic liquid for administration as an implanted depot providing modified release of the active compound. Examples of such formulations include drug-coated stents and semi-solids and suspensions comprising drug-loaded poly(DL-lactic-coglycolic)acid (PGLA) microspheres.

Compound (I) may also be administered topically, intradermally, or transdermally to the skin or mucosa. Typical formulations for this purpose include gels, hydrogels, lotions, solutions, creams, ointments, dusting powders, dressings, foams, films, skin patches, wafers, implants, sponges, fibers, bandages and microemulsions. Liposomes may also be used. Typical carriers may include alcohol, water, mineral oil, liquid petrolatum, white petrolatum, glycerin, polyethylene glycol and propylene glycol. Topical formulations may also include penetration enhancers. See, e.g., Finnin and Morgan, J. Pharm. Sci. 88(10):955-958 (1999).

Other means of topical administration include delivery by electroporation, iontophoresis, phonophoresis, sonophoresis and microneedle or needle-free (e.g., Powderject™ and Bioject™) injection. Formulations for topical administration may be formulated to be immediate or modified release as described above.

Compound (I) may also be administered intranasally or by inhalation, typically in the form of a dry powder, an aerosol spray, or nasal drops. An inhaler may be used to administer the dry powder, which comprises the API alone, a powder blend of the API and a diluent, such as lactose, or a mixed component particle that includes the API and a phospholipid, such as phosphatidylcholine. For intranasal use, the powder may include a bioadhesive agent, e.g., chitosan or cyclodextrin. A pressurized container, pump, sprayer, atomizer, or nebulizer, may be used to generate the aerosol spray from a solution or suspension comprising the API, one or more agents for dispersing, solubilizing, or extending the release of the API (e.g., EtOH with or without water), one or more solvents (e.g., 1,1,1,2-tetrafluoroethane or 1,1,1,2,3,3,3-heptafluoropropane) which serve as a propellant, and an optional surfactant, such as sorbitan trioleate, oleic acid, or an oligolactic acid. An atomizer using electrohydrodynamics may be used to produce a fine mist.

Prior to use in a dry powder or suspension formulation, the drug product is usually comminuted to a particle size suitable for delivery by inhalation (typically 90% of the particles, based on volume, having a largest dimension less than 5 microns). This may be achieved by any appropriate size reduction method, such as spiral jet milling, fluid bed jet milling, supercritical fluid processing, high pressure homogenization, or spray drying.

Capsules, blisters and cartridges (made, for example, from gelatin or hydroxypropylmethyl cellulose) for use in an inhaler or insufflator may be formulated to contain a powder mixture of the active compound, a suitable powder base such as lactose or starch, and a performance modifier such as L-leucine, mannitol, or magnesium stearate. The lactose may be anhydrous or monohydrated. Other suitable excipients include dextran, glucose, maltose, sorbitol, xylitol, fructose, sucrose, and trehalose.

A suitable solution formulation for use in an atomizer using electrohydrodynamics to produce a fine mist may contain from about 1 μg to about 20 mg of the API per actuation and the actuation volume may vary from about 1 μL to about 100 μL. A typical formulation may comprise one or more Compound (I), propylene glycol, sterile water, EtOH, and NaCl. Alternative solvents, which may be used instead of propylene glycol, include glycerol and polyethylene glycol.

Formulations for inhaled administration, intranasal administration, or both, may be formulated to be immediate or modified release using, for example, PGLA. Suitable flavors, such as menthol and levomenthol, or sweeteners, such as saccharin or sodium saccharin, may be added to formulations intended for inhaled/intranasal administration.

In the case of dry powder inhalers and aerosols, the dosage unit is determined by means of a valve that delivers a metered amount. Units are typically arranged to administer a metered dose or “puff” containing from about 10 μg to about 1000 μg of the API. The overall daily dose will typically range from about 100 μg to about 10 mg which may be administered in a single dose or, more usually, as divided doses throughout the day.

The active compounds may be administered rectally or vaginally, e.g., in the form of a suppository, pessary, or enema. Cocoa butter is a traditional suppository base, but various alternatives may be used as appropriate. Formulations for rectal or vaginal administration may be formulated to be immediate or modified release as described above.

Compound (I) may also be administered directly to the eye or ear, typically in the form of drops of a micronized suspension or solution in isotonic, pH-adjusted, sterile saline. Other formulations suitable for ocular and aural administration include ointments, gels, biodegradable implants (e.g., absorbable gel sponges, collagen), non-biodegradable implants (e.g., silicone), wafers, lenses, and particulate or vesicular systems, such as niosomes or liposomes. The formulation may include one or more polymers and a preservative, such as benzalkonium chloride. Typical polymers include crossed-linked polyacrylic acid, polyvinylalcohol, hyaluronic acid, cellulosic polymers (e.g., hydroxypropylmethylcellulose, hydroxyethylcellulose, methyl cellulose), and heteropolysaccharide polymers (e.g., gelan gum). Such formulations may also be delivered by iontophoresis. Formulations for ocular or aural administration may be formulated to be immediate or modified release as described above.

To improve their solubility, dissolution rate, taste-masking, bioavailability, or stability, Compound (I) may be combined with soluble macromolecular entities, including cyclodextrin and its derivatives and polyethylene glycol-containing polymers. For example, API-cyclodextrin complexes are generally useful for most dosage forms and routes of administration. Both inclusion and non-inclusion complexes may be used. As an alternative to direct complexation with the API, the cyclodextrin may be used as an auxiliary additive, i.e., as a carrier, diluent, or solubilizer. Alpha-, beta- and gamma-cyclodextrins are commonly used for these purposes. See, e.g., WO 91/11172, WO 94/02518, and WO 98/55148.

As noted above, one or more compounds of Formula (I), including compounds specifically named above, and their pharmaceutically active complexes, salts, solvates and hydrates, may be combined with each other or with one or more other pharmaceutically active compounds to treat various diseases, conditions and disorders. In such cases, the compounds may be combined in a single dosage form as described above or may be provided in the form of a kit which is suitable for coadministration of the compositions. The kit comprises (1) two or more different pharmaceutical compositions, at least one of which contains Compound (I); and (2) a device for separately retaining the two pharmaceutical compositions, such as a divided bottle or a divided foil packet. An example of such a kit is the familiar blister pack used for the packaging of tablets or capsules. The kit is suitable for administering different types of dosage forms (e.g., oral and parenteral) or for administering different pharmaceutical compositions at separate dosing intervals, or for titrating the different pharmaceutical compositions against one another. To assist with patient compliance, the kit typically comprises directions for administration and may be provided with a memory aid.

For administration to human patients, the total daily dose of the claimed and disclosed compounds is typically in the range of about 0.1 mg to about 3000 mg depending on the route of administration. For example, oral administration may require a total daily dose of from about 1 mg to about 3000 mg, while an intravenous dose may only require a total daily dose of from about 0.1 mg to about 300 mg. The total daily dose may be administered in single or divided doses and, at the physician's discretion, may fall outside of the typical ranges given above. Although these dosages are based on an average human subject having a mass of about 60 kg to about 70 kg, the physician will be able to determine the appropriate dose for a patient (e.g., an infant) whose mass falls outside of this weight range.

As noted above, Compound (I) may be used to treat diseases, disorders and/or conditions associated with NLRP3, i.e., diseases, disorders and/or conditions for which inhibition of the NLRP3 inflammasome pathway is indicated, including diseases, disorders and/or conditions associated with a heterozygous gain of function mutation in the NLRP3 gene, such as a cryopyrin-associated periodic syndrome (CAPS). These may include neonatal-onset multisystem inflammatory disease (NOMID/CINCA), Muckle-Wells syndrome (MWS), and familial cold autoinflammatory syndrome (FCAS).

Compound (I) may be used to treat neurodegenerative diseases and/or conditions associated with NLRP3. These may include Parkinson's disease, Alzheimer's disease, Huntington's disease, amyotrophic lateral sclerosis, prion disease and other forms of dementia (i.e., major or mild neurocognitive disorders) associated with one or more medical conditions, including frontotemporal lobar degeneration, Lewy body disease, vascular disease, traumatic brain injury, substance or medication use, HIV infection, prion disease, Parkinson's disease, and Huntington's disease. Compound (I) may also be used to treat major or mild neurocognitive disorders associated with depression, schizophrenia, bipolar disorder, and autism.

The claimed and disclosed compounds may be combined with one or more other pharmacologically active compounds or therapies to treat one or more disorders, diseases and/or conditions for which inhibition of the NLRP3 inflammasome pathway is indicated. Such combinations may offer significant therapeutic advantages, including fewer side effects, improved ability to treat underserved patient populations, or synergistic activity. For example, compounds of Formula (I), which include compounds specifically named above, and their pharmaceutically acceptable complexes, salts, solvates and hydrates, may be administered simultaneously, sequentially or separately in combination with one or more pharmacologically active compound(s) or therapies for treating Alzheimer's disease, including beta-secretase inhibitors, gamma-secretase inhibitors, HMG-CoA reductase inhibitors, nonsteroidal anti-inflammatory drugs (NSAIDs, such as apazone, aspirin, celecoxib, diclofenac (with and without misoprostol), diflunisal, etodolac, fenoprofen, flurbiprofen, ibuprofen, indomethacin, ketoprofen, meclofenamate sodium, mefenamic acid, meloxicam, nabumetone, naproxen, oxaprozin, phenylbutazone, piroxicam, choline and magnesium salicylates, salsalate, and sulindac), vitamin E, and anti-amyloid antibodies. Specific examples of compounds used to treat Alzheimer's disease include donepezil, rivastigmine, memantine, and galantamine.

In addition to drugs used to improve cognition, Compound (I) may be combined with sedatives, hypnotics, anxiolytics, antipsychotics, tranquilizers, and other medications that are used in the treatment of Alzheimer's disease. For example, Compound (I) may be combined with one or more pharmacologically active agent(s) for treating depression (antidepressants) and/or schizophrenia (atypical or typical antipsychotics) including amitriptyline, amoxapine, aripiprazole, asenapine, bupropion, chlordiazepoxide, citalopram, chlorpromazine, clozapine, desipramine, desvenlafaxine, doxepin, duloxetine, escitalopram, fluoxetine, fluoxetine, fluphenazine, haloperidol, iloperidone, imipramine, isocarboxazid, lamotrigine, levomilnacipran, lurasidone, mirtazapine, nefazodone, nortriptyline, olanzapine, paliperidone, paroxetine, perphenazine, phenelzine, protriptyline, quetiapine, risperidone, selegiline, sertraline, tranylcypromine, trazodone, trimipramine, venlafaxine, vilazodone, and vortioxetine, and ziprasidone.

Likewise, Compound (I) may be combined with one or more pharmaceutically active agent(s) for treating anxiety (anxiolytics) including benzodiazepines (alprazolam, chlordiazepoxide, clobazepam, clonazepam, clorazepate, diazepam, estazolam, flurazepam, lorazepam, midazolam, oxazepam, prazepam, quazepam, temazepam, and triazolam), antihistamines (hydroxyzine), non-benzodiazepines (eszopiclone, zaleplon, zolpidem, and zopiclone) and buspirone.

Compound (I) may also be combined with one or more pharmaceutically active agents for treating epilepsy (antiepileptics or anticonvulsants) including acetazolamide, carbamazepine, clobazam, clonazepam, eslicarbazepine acetate, ethosuximide, gabapentin, lacosamide, lamotrigine, levetiracetam, nitrazepam, oxcarbazepine, perampanel, piracetam, phenobarbital, phenytoin, pregabalin, primidone, retigabine, rufinamide, sodium valproate, stiripentol, tiagabine, topiramate, vigabatrin, and zonisamide.

### EXAMPLES

The following examples are intended to be illustrative and non-limiting and represent specific embodiments of the present invention.

In the following Examples, the “room temperature” generally means about 10 °C to about 35 °C. The ratios indicated for mixed solvents are volume mixing ratios, unless otherwise specified. % means wt %, unless otherwise specified.

The elution by column chromatography in the Examples was performed under the observation by TLC (Thin Layer Chromatography) unless otherwise specified. In the observation by TLC, 60 F₂₅₄ manufactured by Merck was used as a TLC plate, the solvent used as an elution solvent in column chromatography was used as a developing solvent, and UV detector was used for the detection.

In silica gel column chromatography, the indication of NH means use of aminopropylsilane-bonded silica gel and the indication of Diol means use of 3-(2,3-dihydroxypropoxy)propylsilane-bonded silica gel.

¹H Nuclear magnetic resonance (NMR) spectra were obtained for many of the compounds in the following examples. Characteristic chemical shifts (6) are given in parts-per-million downfield from tetramethylsilane using conventional abbreviations for designation of major peaks, including s (singlet), d (doublet), t (triplet), q (quartet), m (multiplet), and br (broad). The following abbreviations are used for common solvents: CDCl₃ (deuterochloroform), DMSO-d₆ (deuterodimethyl sulfoxide), CD₃OD (deuteromethanol), CD₃CN (deuteroacetonitrile), and THF-d₈ (deuterotetrahydrofuran). The mass spectra (m/z for [M+H]⁺) were recorded using electrospray ionization (ESI-MS) or atmospheric pressure chemical ionization (APCI-MS) mass spectrometry. For the analysis of ¹H NMR, ACD/SpecManager (trade name) software and the like were used. Peaks of a hydroxyl group, an amino group and the like, having very mild proton peak, are not sometimes described.

MS was measured by LC/MS. As the ionization method, ESI method, or APCI method was used. The data indicates actual measured value (found). While molecular ion peak is generally observed, a fragment ion is sometimes observed. For example, in the case of a compound having a tert-butoxycarbonyl group, a peak after elimination of a tert-butoxycarbonyl group or a tert-butyl group may be observed as a fragment ion. In the case of a compound having a hydroxy group, a peak after elimination of H₂O may be observed as a fragment ion. In the case of a salt, a molecular ion peak or fragment ion peak of free form is generally observed.

In Examples, the following abbreviations are used.
MS: mass spectrum
M: mol concentration
N: normality
CDCl₃: deuterochloroform
DMSO-d₆: deuterodimethyl sulfoxide
¹H NMR: proton nuclear magnetic resonance
LC/MS: liquid chromatograph mass spectrometer
ESI: electrospray ionization
APCI: atmospheric pressure chemical ionization
DMA: N,N-dimethylacetamide
DME: 1,2-dimethoxyethane
DMF: N,N-dimethylformamide
DMSO: dimethyl sulfoxide
Et₂O: diethyl ether
EtOH: ethanol
IPE: diisopropyl ether
MeOH: methanol
NaH: sodium hydride
Pd(dppf)Cl₂: 1,1′-bis(diphenylphosphino)ferrocene-palladium(II) dichloride
Pd(dppf)Cl₂—CH₂Cl₂: 1,1′-bis(diphenylphosphino)ferrocene-palladium(II) dichloride-dichloromethane adduct
TEA: triethylamine
TFA: trifluoroacetic acid
THF: tetrahydrofuran

### Example 3: 2-(5-{[(3R,5R)-5-fluoro-1-methylpiperidin-3-yl]amino}-2H-pyrazolo[3,4-d][1,3]thiazol-2-yl)-3,5-dimethylphenol

### A) tert-butyl [(3R,5R)-5-fluoro-1-methylpiperidin-3-yl]carbamate

To a mixture of tert-butyl [(3R,5R)-5-fluoropiperidin-3-yl]carbamate (1 g), formaldehyde (1.270 ml) and MeOH (10 ml) was added sodium triacetoxyborohydride (2.428 g) at 0 °C. The mixture was stirred at room temperature for 67 hr. To the mixture was added saturated aqueous sodium hydrogencarbonate solution at 0 °C. The mixture was extracted with ethyl acetate. The organic layer was separated, washed with brine, dried over anhydrous sodium sulfate and concentrated under reduced pressure. The residue was purified by silica gel column chromatography (NH, ethyl acetate/hexane) to give the title compound (1.028 g).

¹H NMR (300 MHz, DMSO-d₆) δ 1.27-1.55 (1H, m), 1.38 (9H, s), 1.71 (1H, t, J=10.4 Hz), 1.82-2.11 (2H, m), 2.15 (3H, s), 2.64-2.87 (2H, m), 3.55-3.78 (1H, m), 4.68-5.02 (1H, m), 6.81 (1H, br d, J=7.6 Hz).

### B) (3R,5R)-5-fluoro-1-methylpiperidin-3-amine dihydrochloride

To a mixture of tert-butyl [(3R,5R)-5-fluoro-1-methylpiperidin-3-yl]carbamate (1.028 g) and ethyl acetate (15 ml) was added 4N hydrogen chloride in ethyl acetate (15 ml) at room temperature. The mixture was stirred at room temperature overnight. To the mixture was added IPE (30 ml) at room temperature. The mixture was stirred at room temperature for 30 min. The precipitated solid was collected, and washed with ethyl acetate/IPE to give the title compound (0.848 g).

¹H NMR (300 MHz, DMSO-d₆) δ 1.66-1.96 (1H, m), 2.31-2.46 (1H, m), 2.59-3.14 (4H, m), 3.19-3.96 (4H, m), 5.08-5.42 (1H, m), 8.60 (3H, br s), 10.73 (1H, br s).

### C) 2-iodo-1-(methoxymethoxy)-3,5-dimethylbenzene

To a mixture of 2-iodo-3,5-dimethylphenol (8.1298 g) and DMF (116 ml) was added 60% NaH (1.742 g) at 0 °C. The mixture was stirred at 0 °C under nitrogen atmosphere for 30 min, then methyl chloromethyl ether (2.90 ml) was added to the mixture at 0 °C and the mixture was stirred for 1 hr. To the mixture was added saturated aqueous ammonium chloride solution at 0 °C. The mixture was extracted with ethyl acetate. The organic layers were washed with brine, dried over anhydrous magnesium sulfate, filtered and concentrated under reduced pressure. The residue was purified by silica gel column chromatography (ethyl acetate/hexane) to give the title compound (9.49 g).

¹H NMR (300 MHz, DMSO-d₆) δ 2.23 (3H, s), 2.35 (3H, s), 3.40 (3H, s), 5.23 (2H, s), 6.74 (1H, s), 6.83 (1H, s).

### D) 2-[2-(methoxymethoxy)-4,6-dimethylphenyl]-4,4,5,5-tetramethyl-1,3,2-dioxaborolane

To a degassed mixture of 2-iodo-1-(methoxymethoxy)-3,5-dimethylbenzene (10.57 g), 4,4,4′,4′,5,5,5′,5′-octamethyl-2,2′-bi(1,3,2-dioxaborolane) (11.03 g), potassium acetate (7.10 g) and DMSO (96 ml) was added Pd(dppf)Cl₂—CH₂Cl₂ (1.477 g) at room temperature. The mixture was flushed with nitrogen gas and stirred at 100 °C under nitrogen atmosphere overnight. The mixture was filtered through NH silica gel and Celite® and flushed with ethyl acetate. To the filtrate was added water, and then the mixture was extracted with ethyl acetate. The organic layers were washed with brine, dried over anhydrous magnesium sulfate, filtered and concentrated under reduced pressure. The residue was purified by silica gel column chromatography (NH, ethyl acetate/hexane) to give the title compound (4.32 g).

¹H NMR (300 MHz, DMSO-d₆) δ 1.29 (12H, s), 2.20 (3H, s), 2.22 (3H, s), 3.36 (3H, s), 5.09 (2H, s), 6.61 (1H, s), 6.63 (1H, s).

### E) 4-bromo-1-[2-(methoxymethoxy)-4,6-dimethylphenyl]-3-nitro-1H-pyrazole

To a mixture of 4-bromo-5-nitro-1H-pyrazole (0.282 g), 2-[2-(methoxymethoxy)-4,6-dimethylphenyl]-4,4,5,5-tetramethyl-1,3,2-dioxaborolane (0.3585 g), pyridine (0.792 ml), and DMA (7.18 ml) was added copper(II) acetate (0.447 g) at room temperature. The mixture was stirred at 90 °C overnight. The mixture was poured into saturated aqueous ammonium chloride solution and ethyl acetate at 0 °C. The mixture was stirred at room temperature for 1 hr. The insoluble materials were removed by filtration. The filtrate was extracted with ethyl acetate. The organic layer was separated, washed with saturated aqueous ammonium chloride solution and brine, dried over anhydrous magnesium sulfate and concentrated under reduced pressure. The residue was purified by silica gel column chromatography (ethyl acetate/hexane) to give the title compound (0.299 g). MS: [M+Na]⁺ 377.9.

### F) 4-bromo-1-[2-(methoxymethoxy)-4,6-dimethylphenyl]-1H-pyrazol-3-amine

To a mixture of 4-bromo-1-[2-(methoxymethoxy)-4,6-dimethylphenyl]-3-nitro-1H-pyrazole (1.72 g), EtOH (25.8 ml) and water (6.44 ml) were added iron (1.618 g) and ammonium chloride (1.033 g) at room temperature. The mixture was stirred at 80 °C for 1 hr. The insoluble materials were removed by filtration through Celite®, and washed with EtOH. The filtrate was concentrated under reduced pressure. To the mixture was added saturated aqueous sodium hydrogencarbonate. The mixture was extracted with ethyl acetate. The organic layer was separated, washed with brine, dried over anhydrous magnesium sulfate and concentrated under reduced pressure. The residue was purified by silica gel column chromatography (NH, ethyl acetate/hexane) to give the title compound (1.11 g). MS: [M+H]⁺ 325.9.

### G) N-[(3R,5R)-5-fluoro-1-methylpiperidin-3-yl]-2-[2-(methoxymethoxy)-4,6-dimethylphenyl]-2H-pyrazolo[3,4-d][1,3]thiazol-5-amine

To a mixture of 4-bromo-1-[2-(methoxymethoxy)-4,6-dimethylphenyl]-1H-pyrazol-3-amine (3 g), triethylamine (12.82 ml) and THE (60.1 ml) was added thiophosgene (0.917 ml) at 0 °C. The mixture was stirred at 0 °C under nitrogen atmosphere for 2 hr. To the mixture was added (3R,5R)-5-fluoro-1-methylpiperidin-3-amine dihydrochloride (3.1 g) at 0 °C. The mixture was stirred at room temperature overnight. To the mixture was added water at room temperature. The mixture was extracted with ethyl acetate. The organic layer was separated, washed with brine, dried over anhydrous sodium sulfate and concentrated under reduced pressure to give a brown solid (5.61 g). To a mixture of the solid (0.5 g), 1,10-phenanthroline (0.014 g), cesium carbonate (0.651 g) and DME (12.98 ml) was added copper(I) iodide (9.51 mg) at room temperature. The mixture was heated to 80 °C and stirred at 80 °C for 3 hr. The mixture was filtered through Celite®. The filtrate was concentrated under reduced pressure. The residue was purified by silica gel column chromatography (NH, ethyl acetate/hexane) to give the title compound (0.260 g). MS: [M+H]⁺ 420.0.

### H) 2-(5-{[(3R,5R)-5-fluoro-1-methylpiperidin-3-yl]amino}-2H-pyrazolo[3,4-d][1,3]thiazol-2-yl)-3,5-dimethylphenol

To a mixture of N-[(3R,5R)-5-fluoro-1-methylpiperidin-3-yl]-2-[2-(methoxymethoxy)-4,6-dimethylphenyl]-2H-pyrazolo[3,4-d][1,3]thiazol-5-amine (0.35 g) and MeOH (2 ml) was added 4N hydrogen chloride in ethyl acetate (4 ml) at 0 °C. The mixture was stirred at room temperature for 1 hr. The mixture was quenched with saturated aqueous sodium hydrogencarbonate at 0 °C and extracted with ethyl acetate. The organic layers were dried over anhydrous magnesium sulfate, filtered and concentrated under reduced pressure. The residue was purified by silica gel column chromatography (NH, ethyl acetate/hexane, and then MeOH/ethyl acetate) to give a yellow solid. To a mixture of the solid and ethyl acetate was added ethyl acetate at 80 °C. The mixture was stirred at 80 °C for 30 min. To the mixture was added heptane at 80 °C. The mixture was stirred at 80 °C for 30 min. The mixture was allowed to cool to room temperature, and stirred at room temperature for 66 hr. The precipitated solid was collected, washed with ethyl acetate/heptane, and dried to give the title compound (0.144 g).

¹H NMR (400 MHz, DMSO-d₆) δ 1.54-1.76 (1H, m), 1.93 (3H, s), 1.99 (1H, br t, J=9.5 Hz), 2.06-2.18 (1H, m), 2.19-2.33 (1H, m), 2.22 (3H, s), 2.24 (3H, s), 2.71-2.81 (1H, m), 2.86-2.96 (1H, m), 4.04-4.23 (1H, m), 4.71-5.08 (1H, m), 6.58 (1H, s), 6.63 (1H, s), 7.58 (1H, s), 8.01 (1H, d, J=7.7 Hz), 9.61 (1H, br s).

### Example 4: 3,5-dimethyl-2-(5-{[(3R)-1-methylpiperidin-3-yl]amino}-2H-pyrazolo[3,4-d][1,3]thiazol-2-yl)phenol

### A) 2-[2-(methoxymethoxy)-4,6-dimethylphenyl]-N-[(3R)-1-methylpiperidin-3-yl]-2H-pyrazolo[3,4-d][1,3]thiazol-5-amine

To a mixture of 4-bromo-1-[2-(methoxymethoxy)-4,6-dimethylphenyl]-1H-pyrazol-3-amine (65.3 mg), TEA (0.279 ml) and THE (2 ml) was added thiophosgene (0.020 ml) at 0 °C. The mixture was stirred at 0 °C under nitrogen atmosphere for 1 hr. To the mixture was added (R)-1-methylpiperidin-3-amine dihydrochloride (74.9 mg) at 0 °C. The mixture was stirred at room temperature overnight. To the mixture was added water at room temperature, the mixture was extracted with ethyl acetate. The organic layer was separated, washed with brine, dried over anhydrous sodium sulfate and concentrated under reduced pressure. To a mixture of the residue, 1,10-phenanthroline (21.6 mg), cesium carbonate (131 mg) and DME (3 mL) was added copper(I) iodide (15.3 mg) at room temperature. The mixture was stirred at 80 °C for 2 hr. The mixture was purified by silica gel column chromatography (NH, ethyl acetate/hexane, and then MeOH/ethyl acetate) to give the title compound (56.8 mg). MS: [M+H]⁺ 402.2.

### B) 3,5-dimethyl-2-(5-{[(3R)-1-methylpiperidin-3-yl]amino}-2H-pyrazolo[3,4-d][1,3]thiazol-2-yl)phenol

To a mixture of 2-[2-(methoxymethoxy)-4,6-dimethylphenyl]-N-[(3R)-1-methylpiperidin-3-yl]-2H-pyrazolo[3,4-d][1,3]thiazol-5-amine (55.3 mg) and thioanisole (0.161 ml) was added TFA (1 ml) at 0 °C. The mixture was stirred at room temperature for 2 hr. The mixture was concentrated under reduced pressure. The residue was purified by silica gel column chromatography (NH, ethyl acetate/hexane, and then MeOH/ethyl acetate) and washed with ethyl acetate/IPE to give the title compound (26.3 mg).

¹H NMR (400 MHz, DMSO-d₆) δ 1.22-1.38 (1H, m), 1.44-1.60 (1H, m), 1.64-1.77 (1H, m), 1.81-2.05 (6H, m), 2.18 (3H, s), 2.23 (3H, s), 2.52-2.58 (1H, m), 2.80-2.94 (1H, m), 3.77-3.92 (1H, m), 6.57 (1H, s), 6.63 (1H, s), 7.56 (1H, s), 7.95 (1H, d, J=7.5 Hz), 9.56 (1H, br s).

### Example 20: 3-ethyl-2-(5-{[(3R,5R)-5-fluoro-1-methylpiperidin-3-yl]amino}-2H-pyrazolo[3,4-d][1,3]thiazol-2-yl)-5-methylphenol

### A) 1,2-bis(methoxymethoxy)-4-methylbenzene

60% NaH (1.853 g) was added to a mixture of 4-methylbenzene-1,2-diol (2.3 g) and DMF (30 ml) at 0 °C. After being stirred at 0 °C for 5 min, chloromethyl methyl ether (4.22 ml) was added to the mixture. The mixture was stirred at room temperature under nitrogen atmosphere for 1 hr. The mixture was poured into saturated aqueous ammonium chloride solution at room temperature and extracted with ethyl acetate. The organic layer was separated, washed with water and brine, dried over anhydrous magnesium sulfate and concentrated under reduced pressure. The residue was purified by silica gel column chromatography (ethyl acetate/hexane) to give the title compound (3.80 g).

¹H NMR (400 MHz, CDCl₃) δ 2.28 (3H, s), 3.51 (3H, s), 3.52 (3H, s), 5.19 (2H, s), 5.21 (2H, s), 6.76 (1H, dd, J=8.31, 1.83 Hz), 6.98 (1H, d, J=1.83 Hz), 7.04 (1H, d, J=8.31 Hz).

### B) 2,3-bis(methoxymethoxy)-5-methylbenzaldehyde

n-Butyllithium (1.6M in hexane) (15 ml) was added to a mixture of 1,2-bis(methoxymethoxy)-4-methylbenzene (3.50 g) and Et₂O (30 ml) at 0 °C. After being stirred at 0 °C for 10 min, DMF (3 ml) was added to the mixture. The mixture was stirred at 0 °C under nitrogen atmosphere for 1 hr. The mixture was poured into water at room temperature and extracted with ethyl acetate. The organic layer was separated, washed with water and brine, dried over anhydrous magnesium sulfate and concentrated under reduced pressure. The residue was purified by silica gel column chromatography (ethyl acetate/hexane) to give the title compound (2.75 g).

¹H NMR (400 MHz, CDCl₃) δ 2.33 (3H, s), 3.52 (3H, s), 3.57 (3H, s), 5.21 (2H, s), 5.22 (2H, s), 7.20-7.24 (1H, m), 7.29-7.32 (1H, m), 10.42 (1H, s).

### C) 2-hydroxy-3-(methoxymethoxy)-5-methylbenzaldehyde

Amberlyst® 15, wet, ion exchange resin (12.0 g) was added to a mixture of 2,3-bis(methoxymethoxy)-5-methylbenzaldehyde (31.0 g) and toluene (300 ml) at room temperature. The mixture was stirred at room temperature under nitrogen atmosphere for 1 hr. The insoluble materials were removed by filtration, and the filtrate was concentrated under reduced pressure. The residue was purified by column chromatography (Diol, ethyl acetate/hexane) to give the title compound (16.20 g). MS: [M−H]⁻ 194.8.

### D) 2-(benzyloxy)-3-(methoxymethoxy)-5-methylbenzaldehyde

60% NaH (1.427 g) was added to a mixture of 2-hydroxy-3-(methoxymethoxy)-5-methylbenzaldehyde (7.0 g) and DMF (200 ml) at 0 °C. After being stirred at 0 °C for 10 min, benzyl bromide (4.23 ml) was added to the mixture. The mixture was stirred at room temperature under nitrogen atmosphere for 1 hr. The mixture was poured into saturated aqueous ammonium chloride solution at room temperature and extracted with ethyl acetate. The organic layer was separated, washed with water and brine, dried over anhydrous magnesium sulfate and concentrated under reduced pressure. The residue was purified by silica gel column chromatography (ethyl acetate/hexane) to give the title compound (4.50 g).

¹H NMR (300 MHz, CDCl₃) δ 2.33 (3H, s), 3.55 (3H, s), 5.14 (2H, s), 5.26 (2H, s), 7.22-7.44 (7H, m), 10.18 (1H, s).

### E) 2-(benzyloxy)-1-ethenyl-3-(methoxymethoxy)-5-methylbenzene

n-Butyllithium (1.6M in hexane) (12.77 ml) was added to a mixture of methyltriphenylphosphonium bromide (8.42 g) and THE (50 ml) at 0 °C. After being stirred at 0 °C for 20 min, 2-(benzyloxy)-3-(methoxymethoxy)-5-methylbenzaldehyde (4.50 g) was added to the mixture. The mixture was stirred at 0 °C under nitrogen atmosphere for 1 hr. The mixture was poured into saturated aqueous ammonium chloride solution at room temperature and extracted with ethyl acetate. The organic layer was separated, washed with water and brine, dried over anhydrous magnesium sulfate and concentrated under reduced pressure. The residue was purified by silica gel column chromatography (ethyl acetate/hexane) to give the title compound (3.78 g). MS: [M−H]⁻ 283.1.

### F) 2-ethyl-6-(methoxymethoxy)-4-methylphenol

A mixture of 2-(benzyloxy)-1-ethenyl-3-(methoxymethoxy)-5-methylbenzene (2.6 g) and 10% palladium-carbon (0.973 g) in EtOH (30 ml) was stirred under hydrogen atmosphere at room temperature overnight. The catalyst was removed by filtration and the filtrate was concentrated under reduced pressure. The residue was purified by silica gel column chromatography (ethyl acetate/hexane) to give the title compound (1.75 g). MS: [M−H]⁻ 195.0.

### G) 2-ethyl-6-(methoxymethoxy)-4-methylphenyl trifluoromethanesulfonate

Cesium carbonate (6.0 g) was added to a mixture of 2-ethyl-6-(methoxymethoxy)-4-methylphenol (1.75 g) and DMF (20 ml) at 0 °C. After being stirred at 0 °C for 10 min, N-phenyl bis(trifluoromethanesulfonimide) (3.82 g) was added to the mixture. The mixture was stirred at room temperature under nitrogen atmosphere for 2 hr. The mixture was poured into saturated aqueous ammonium chloride solution at room temperature and extracted with ethyl acetate. The organic layer was separated, washed with water and brine, dried over anhydrous magnesium sulfate and concentrated under reduced pressure. The residue was purified by silica gel column chromatography (ethyl acetate/hexane) to give the title compound (2.73 g).

¹H NMR (300 MHz, CDCl₃) δ 1.23 (3H, t, J=7.53 Hz), 2.32 (3H, s), 2.68 (2H, q, J=7.53 Hz), 3.51 (3H, s), 5.20 (2H, s), 6.72-6.74 (1H, m), 6.90-6.94 (1H, m).

### H) 2-[2-ethyl-6-(methoxymethoxy)-4-methylphenyl]-4,4,5,5-tetramethyl-1,3,2-dioxaborolane

A mixture of 2-ethyl-6-(methoxymethoxy)-4-methylphenyl trifluoromethanesulfonate (2.25 g), 4,4,4′,4′,5,5,5′,5′-octamethyl-2,2′-bi(1,3,2-dioxaborolane) (2.262 g), potassium acetate (2.018 g), Pd(dppf)Cl₂ (0.501 g) and DMSO (30 ml) was stirred at 80 °C under argon atmosphere overnight. The mixture was poured into water at room temperature and extracted with ethyl acetate. The organic layer was separated, washed with water and brine, dried over anhydrous magnesium sulfate and concentrated under reduced pressure. The residue was purified by silica gel column chromatography (ethyl acetate/hexane) to give the title compound (0.135 g).

¹H NMR (300 MHz, CDCl₃) δ 1.19 (3H, t, J=7.78 Hz), 1.37 (12H, s), 2.28 (3H, s), 2.59 (2H, q, J=7.78 Hz), 3.47 (3H, s), 5.12 (2H, s), 6.65 (2H, s).

### I) 4-bromo-1-[2-ethyl-6-(methoxymethoxy)-4-methylphenyl]-3-nitro-1H-pyrazole

To a mixture of 4-bromo-3-nitro-1H-pyrazole (169 mg), 2-[2-ethyl-6-(methoxymethoxy)-4-methylphenyl]-4,4,5,5-tetramethyl-1,3,2-dioxaborolane (135 mg), pyridine (0.284 ml) and DMA (3 ml) was added copper(II) acetate (160 mg) at room temperature. The mixture was stirred at 90 °C overnight. The mixture was poured into saturated aqueous ammonium chloride solution and ethyl acetate at 0 °C. The mixture was stirred at room temperature for 1 hr. The insoluble materials were removed by filtration. The filtrate was extracted with ethyl acetate. The organic layer was separated, washed with saturated aqueous ammonium chloride solution and brine, dried over anhydrous sodium sulfate and concentrated under reduced pressure. The residue was purified by silica gel column chromatography (ethyl acetate/hexane) to give the title compound (128 mg). MS: [M+H]⁺ 370.0.

### J) 4-bromo-1-[2-ethyl-6-(methoxymethoxy)-4-methylphenyl]-1H-pyrazol-3-amine

To a mixture of 4-bromo-1-[2-ethyl-6-(methoxymethoxy)-4-methylphenyl]-3-nitro-1H-pyrazole (120 mg), EtOH (24 ml) and water (6 ml) were added iron (109 mg) and ammonium chloride (69.4 mg) at room temperature. The mixture was stirred at 80 °C for 1 hr. The insoluble materials were removed by filtration through Celite®, and washed with EtOH. The filtrate was concentrated under reduced pressure. To the mixture was added saturated aqueous sodium hydrogencarbonate, and the mixture was extracted with ethyl acetate. The organic layer was separated, washed with brine, dried over anhydrous sodium sulfate and concentrated under reduced pressure. The residue was purified by silica gel column chromatography (NH, ethyl acetate/hexane) to give the title compound (83 mg). MS: [M+H]⁺ 340.0.

### K) 2-[2-ethyl-6-(methoxymethoxy)-4-methylphenyl]-N-[(3R,5R)-5-fluoro-1-methylpiperidin-3-yl]-2H-pyrazolo[3,4-d][1,3]thiazol-5-amine

To a mixture of 4-bromo-1-[2-ethyl-6-(methoxymethoxy)-4-methylphenyl]-1H-pyrazol-3-amine (83 mg), TEA (0.340 ml) and THE (1.5 ml) was added thiophosgene (0.024 ml) at 0 °C. The mixture was stirred at 0 °C under nitrogen atmosphere for 1 hr. To the mixture was added (3R,5R)-5-fluoro-1-methylpiperidin-3-amine dihydrochloride (85 mg) at 0 °C. The mixture was stirred at room temperature overnight. To the mixture was added water at room temperature. The mixture was extracted with ethyl acetate. The organic layer was separated, washed with brine, dried over anhydrous sodium sulfate and concentrated under reduced pressure. To a mixture of the residue, 1,10-phenanthroline (25.9 mg), cesium carbonate (156 mg) and DME (3 ml) was added copper(I) iodide (18.28 mg) at room temperature. The mixture was stirred at 80 °C for 2 hr. The mixture was purified by silica gel column chromatography (NH, ethyl acetate/hexane, and then MeOH/ethyl acetate) to give the title compound (68.0 mg). MS: [M+H]⁺ 434.2.

### L) 3-ethyl-2-(5-{[(3R,5R)-5-fluoro-1-methylpiperidin-3-yl]amino}-2H-pyrazolo[3,4-d][1,3]thiazol-2-yl)-5-methylphenol

To a mixture of 2-[2-ethyl-6-(methoxymethoxy)-4-methylphenyl]-N-[(3R,5R)-5-fluoro-1-methylpiperidin-3-yl]-2H-pyrazolo[3,4-d][1,3]thiazol-5-amine (68 mg) and thioanisole (0.184 ml) was added TFA (1 ml) at 0 °C. The mixture was stirred at room temperature for 1 hr. The mixture was concentrated under reduced pressure. The residue was purified by silica gel column chromatography (NH, MeOH/ethyl acetate) to give the title compound (46.0 mg).

¹H NMR (300 MHz, CDCl₃) δ 1.09-1.17 (3H, m), 2.00-2.29 (3H, m), 2.30-2.33 (3H, m), 2.34-2.37 (3H, m), 2.37-2.76 (5H, m), 2.82-2.94 (1H, m), 4.01-4.20 (1H, m) 4.75-4.99 (1H, m), 6.66-6.70 (1H, m), 6.71-6.76 (1H, m), 7.46 (1H, s), 7.63-7.88 (1H, m).

The compounds of Examples are shown in Table 1-1 to Table 1-18. MS in the tables means actual measured value. The compounds of Examples 1, 2, 5-19 and 21-181 in the following tables were produced according to the methods described in the above-mentioned Examples, or methods analogous thereto.

TABLE 1-1

| Ex- | | | |
|---|---|---|---|
| ample | | | |
| No. | Compound name | Structure | MS |
| 1 | 5-bromo-2-(5-{[(3R)-1-methylpiperidin-3-yl]amino}-2H- pyrazolo[3,4-d][1,3]thiazol-2-yl)phenol | 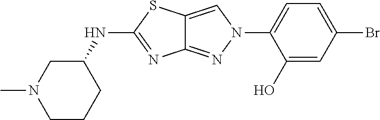 [SMILES: CN1CCC[C@@H](Nc2nc3nn(-c4ccc(Br)cc4O)cc3s2)C1] | 408.1 |
| 2 | 5-chloro-3-methyl-2-(5-{[(3R)-1-methylpiperidin-3- yl]amino}-2H-pyrazolo[3,4-d][1,3]thiazol-2-yl)phenol | 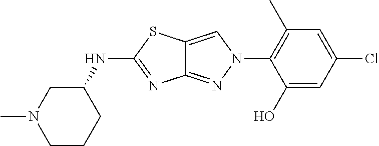 [SMILES: Cc1cc(Cl)cc(O)c1-n1cc2sc(N[C@@H]3CCCN(C)C3)nc2n1] | 378.1 |
| 3 | 2-(5-{[(3R,5R)-5-fluoro-1-methylpiperidin-3-yl]amino}-2H- pyrazolo[3,4-d][1,3]thiazol-2-yl)-3,5-dimethylphenol | 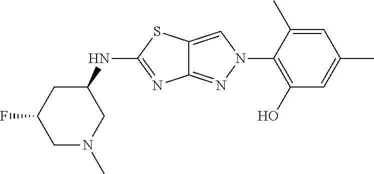 [SMILES: Cc1cc(C)c(-n2cc3sc(N[C@@H]4C[C@@H](F)CN(C)C4)nc3n2)c(O)c1] | 376.1 |
| 4 | 3,5-dimethyl-2-(5-{[(3R)-1-methylpiperidin-3-yl]amino}-2H- pyrazolo[3,4-d][1,3]thiazol-2-yl)phenol | 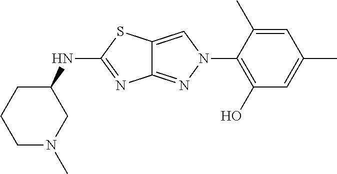 [SMILES: Cc1cc(C)c(-n2cc3sc(N[C@@H]4CCCN(C)C4)nc3n2)c(O)c1] | 358.1 |
| 5 | 5-fluoro-3-methyl-2-(5-{[(3R)-1-methylpiperidin-3-yl]amino}- 2H-pyrazolo[3,4-d][1,3]thiazol-2-yl)phenol | 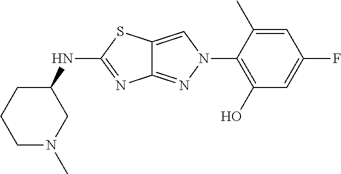 [SMILES: Cc1cc(F)cc(O)c1-n1cc2sc(N[C@@H]3CCCN(C)C3)nc2n1] | 362.1 |
| 6 | 5-fluoro-2-(5-{[(1R,2R)-2-hydroxycyclohexyl]amino}-2H- pyrazolo[3,4-d][1,3]thiazol-2-yl)-3-methylphenol | 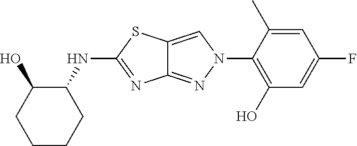 [SMILES: Cc1cc(F)cc(O)c1-n1cc2sc(N[C@@H]3CCCC[C@H]3O)nc2n1] | 363.2 |
| 7 | 2-(5-{[(3R,5R)-1-ethyl-5-fluoropiperidin-3-yl]amino}-2H- pyrazolo[3,4-d][1,3]thiazol-2-yl)-3,5-dimethylphenol | 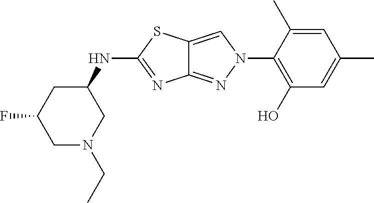 [SMILES: CCN1C[C@H](F)C[C@@H](Nc2nc3nn(-c4c(C)cc(C)cc4O)cc3s2)C1] | 390.2 |
| 8 | 3-chloro-2-(5-{[(3R,5R)-5-fluoro-1-methylpiperidin-3- yl]amino}-2H-pyrazolo[3,4-d][1,3]thiazol-2-yl)phenol | 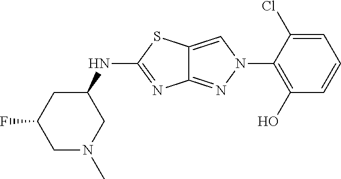 [SMILES: CN1C[C@H](F)C[C@@H](Nc2nc3nn(-c4c(O)cccc4Cl)cc3s2)C1] | 382.0 |
| 9 | 5-chloro-2-(5-{[(3R,5R)-5-fluoro-1-methylpiperidin-3- yl]amino}-2H-pyrazolo[3,4-d][1,3]thiazol-2-yl)-3- methylphenol | 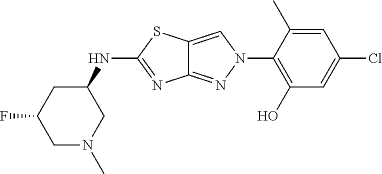 [SMILES: Cc1cc(Cl)cc(O)c1-n1cc2sc(N[C@@H]3C[C@@H](F)CN(C)C3)nc2n1] | 396.1 |
| 10 | 2-(5-{[(3S,4S)-4-fluoro-1-methylpiperidin-3-yl]amino}-2H- pyrazolo[3,4-d][1,3]thiazol-2-yl)-3,5-dimethylphenol | 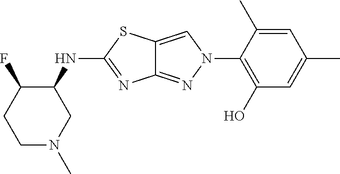 [SMILES: Cc1cc(C)c(-n2cc3sc(N[C@H]4CN(C)CC[C@H]4F)nc3n2)c(O)c1] | 376.1 |

TABLE 1-2

| | | | |
|---|---|---|---|
| 11 | 3-chloro-2-(5-{[(3R,5R)-5-fluoro-1-methylpiperidin- 3-yl]amino}-2H-pyrazolo[3,4-d][1,3]thiazol-2-yl)-5- methylphenol | 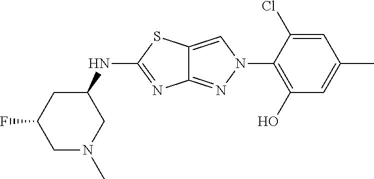 [SMILES: Cc1cc(O)c(-n2cc3sc(N[C@@H]4C[C@@H](F)CN(C)C4)nc3n2)c(Cl)c1] | 396.1 |
| 12 | 2-(5-{[(3R)-1-cyclopropylpiperidin-3-yl]amino}-2H- pyrazolo[3,4-d][1,3]thiazol-2-yl)-5-fluoro-3- methylphenol | 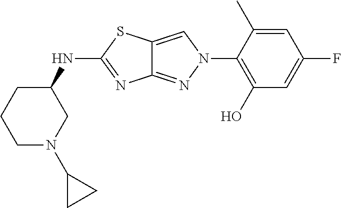 [SMILES: Cc1cc(F)cc(O)c1-n1cc2sc(N[C@@H]3CCCN(C4CC4)C3)nc2n1] | 388.1 |
| 13 | 5-(difluoromethyl)-2-(5-{[(3R,5R)-5-fluoro-1- methylpiperidin-3-yl]amino}-2H- pyrazolo[3,4-d][1,3]thiazol- 2-yl)-3-methylphenol | 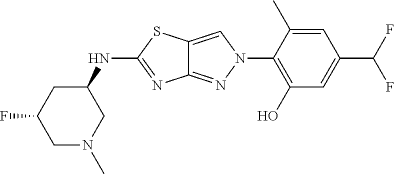 [SMILES: Cc1cc(C(F)F)cc(O)c1-n1cc2sc(N[C@@H]3C[C@@H](F)CN(C)C3)nc2n1] | 412.2 |
| 14 | 2-(5-{[(3R,4S)-4-fluoro-1-methyl- piperidin-3-yl]amino}-2H-pyrazolo[3,4- d][1,3]thiazol-2-yl)-3-methylphenol or 2-(5- {[(3S,4R)-4-fluoro-1-methylpiperidin-3-yl]amino}- 2H-pyrazolo[3,4-d][1,3]thiazol-2-yl)- 3-methylphenol (tR1) | 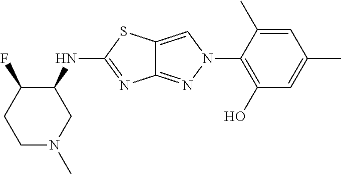 [SMILES: Cc1cc(C)c(-n2cc3sc(N[C@H]4CN(C)CC[C@H]4F)nc3n2)c(O)c1] | 376.2 |
| 15 | 2-(5-{[(3R,4S)-4-fluoro-1-methyl- piperidin-3-yl]amino}-2H-pyrazolo[3,4- d][1,3]thiazol-2-yl)-3-methylphenol or 2-(5- {[(3S,4R)-4-fluoro-1-methylpiperidin-3- yl]amino}-2H-pyrazolo[3,4-d][1,3]thiazol- 2-yl)-3-methylphenol (tR2) | 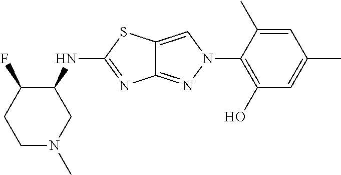 [SMILES: Cc1cc(C)c(-n2cc3sc(N[C@H]4CN(C)CC[C@H]4F)nc3n2)c(O)c1] | 376.2 |
| 16 | 5-(difluoromethyl)-2-(5-{[(3R,5R)-1- ethyl-5-fluoropiperidin-3-yl]amino}-2H- pyrazolo[3,4-d][1,3]thiazol-2-yl)-3-methylphenol | 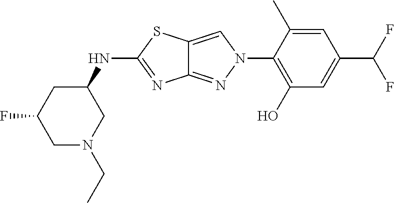 [SMILES: CCN1C[C@H](F)C[C@@H](Nc2nc3nn(-c4c(C)cc(C(F)F)cc4O)cc3s2)C1] | 426.1 |
| 17 | 3-chloro-5-fluoro-2-(5-{[(3R,5R)- 5-fluoro-1-methylpiperidin- 3-yl]amino}-2H-pyrazolo[3,4-d][1,3]thi- azol-2-yl)phenol | 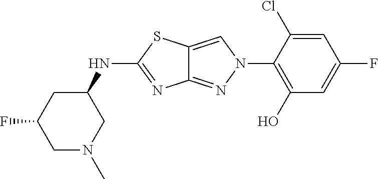 [SMILES: CN1C[C@H](F)C[C@@H](Nc2nc3nn(-c4c(O)cc(F)cc4Cl)cc3s2)C1] | 400.1 |
| 18 | 3-chloro-2-(5-{[(3R)-1-cyclopropyl- piperidin-3-yl]amino}-2H- pyrazolo[3,4-d][1,3]thiazol-2-yl)-5-fluorophenol | 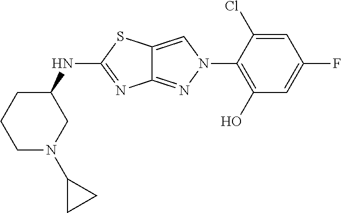 [SMILES: Oc1cc(F)cc(Cl)c1-n1cc2sc(N[C@@H]3CCCN(C4CC4)C3)nc2n1] | 408.1 |
| 19 | 2-(5-{[(3R,5S)-5-methoxy-1- methylpiperidin-3-yl]amino}-2H- pyrazolo[3,4-d][1,3]thiazol-2-yl)-3,5- dimethylphenol | 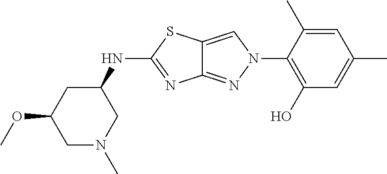 | 388.2 |
| 20 | 3-ethyl-2-(5-{[(3R,5R)-5-fluoro-1- methylpiperidin-3-yl]amino}-2H- pyrazolo[3,4-d][1,3]thiazol-2-yl)-5-methylphenol | 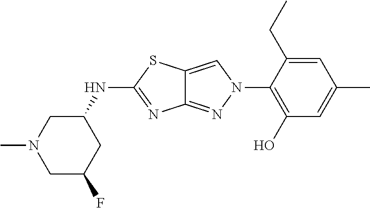 | 390.1 |

TABLE 3

| | | | |
|---|---|---|---|
| 21 | racemic mixture of 2-(5-{[(3R,4R)-1- ethyl-4-fluoropyrrolidin-3- yl]amino}-2H-pyrazolo[3,4-d][1,3]thiazol- 2-yl)-3,5-dimethylphenol and 2-(5- {[(3S,4S)-1-ethyl-4-fluoropyrrolidin-3- yl]amino}-2H-pyrazolo[3,4-d][1,3]thiazol- 2-yl)-3,5-dimethylphenol | 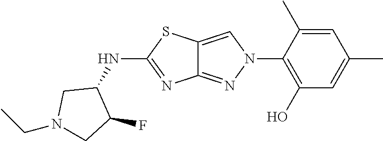 [SMILES: CCN1C[C@H](Nc2nc3nn(-c4c(C)cc(C)cc4O)cc3s2)[C@@H](F)C1] | 376.1 |
| 22 | racemic mixture of 2-(5-{[(3R,4S)- 4-fluoro-1-methylpyrrolidin-3- yl]amino}-2H-1-pyrazolo[3,4- d][1,3]thiazol-2-yl)-3,5-dimethyl- phenol and 2-(5-{[(3S,4R)-4-fluoro- methylpyrrolidin-3-yl]amino}- 2H-pyrazolo[3,4-d][1,3]thiazol-2-yl)- 3,5-dimethylphenol | 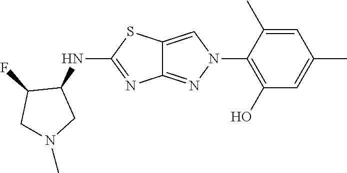 [SMILES: Cc1cc(C)c(-n2cc3sc(N[C@H]4CN(C)C[C@H]4F)nc3n2)c(O)c1] | 362.1 |
| 23 | racemic mixture of 2-(5-{[(3R,4S)-1- ethyl-4-fluoropyrrolidin-3-yl]amino}- 2H-pyrazolo[3,4-d][1,3]thiazol-2-yl)-3,5- dimethylphenol and 2-(5-{[(3S,4R)-1- ethyl-4-fluoropyrrolidin-3-yl]amino}-2H- pyrazolo[3,4-d][1,3]thiazol- 2-yl)-3,5-dimethylphenol | 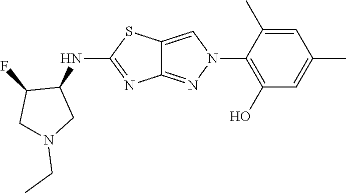 [SMILES: CCN1C[C@@H](F)[C@@H](Nc2nc3nn(-c4c(C)cc(C)cc4O)cc3s2)C1] | 376.1 |
| 24 | 3-(difluoromethyl)-2-(5-{[(3R,5R)- 1-ethyl-5-fluoropiperidin- 3-yl]amino}-2H-pyrazolo[3,4- d][1,3]thiazol-2-yl)-5-fluorophenol | 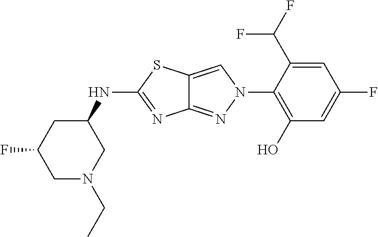 [SMILES: CCN1C[C@H](F)C[C@@H](Nc2nc3nn(-c4c(O)cc(F)cc4C(F)F)cc3s2)C1] | 430.1 |
| 25 | 2-{5-[(trans-4-hydroxycyclo- hexyl)amino]-2H-pyrazolo[3,4- d][1,3]thiazol-2-yl}-3,5-dimethylphenol | 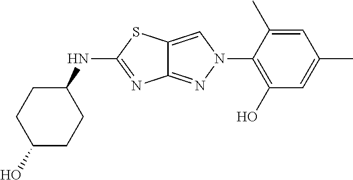 [SMILES: Cc1cc(C)c(-n2cc3sc(N[C@H]4CC[C@H](O)CC4)nc3n2)c(O)c1] | 359.1 |
| 26 | 2-{5-[(cis-4-hydroxycyclohexyl)amino]- 2H-pyrazolo[3,4- d][1,3]thiazol-2-yl}-3,5-dimethylphenol | 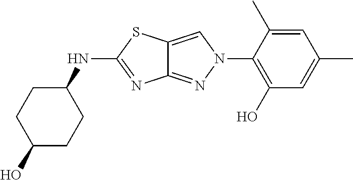 [SMILES: Cc1cc(C)c(-n2cc3sc(N[C@H]4CC[C@@H](O)CC4)nc3n2)c(O)c1] | 359.2 |
| 27 | 2,2,2-trichloroethyl (3R)-3-{[2-(2- hydroxy-4,6-dimethylphenyl)-2H- pyrazolo[3,4-d][1,3]thiazol-5- yl]amino}piperidine-1-carboxylate | 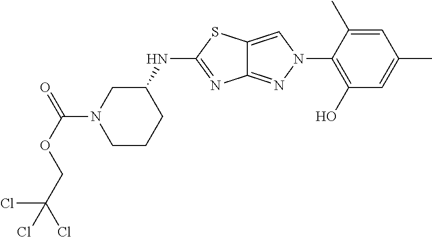 [SMILES: Cc1cc(C)c(-n2cc3sc(N[C@@H]4CCCN(C(=O)OCC(Cl)(Cl)Cl)C4)nc3n2)c(O)c1] | 518.1 |
| 28 | 2-{5-[(2-hydroxycyclopentyl)amino]- 2H-pyrazolo[3,4-d][1,3]thiazol-2-yl}- 3,5-dimethylphenol | 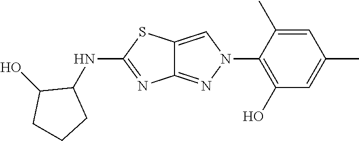 [SMILES: Cc1cc(C)c(-n2cc3sc(NC4CCCC4O)nc3n2)c(O)c1] | 345.2 |
| 29 | 2-{5-[(3-hydroxycyclohexyl)amino]- 2H-pyrazolo[3,4-d][1,3]thiazol- 2-yl}-3,5-dimethylphenol | 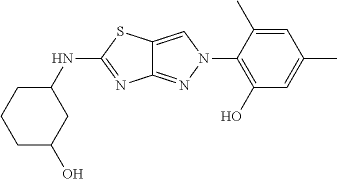 [SMILES: Cc1cc(C)c(-n2cc3sc(NC4CCCC(O)C4)nc3n2)c(O)c1] | 359.2 |
| 30 | 6-{[2-(2-hydroxy-4,6-dimethylphenyl)- 2H-pyrazolo[3,4-d][1,3]thiazol- 5-yl]amino}spiro[3.3]heptan-2-ol | 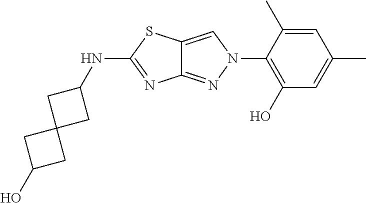 [SMILES: Cc1cc(C)c(-n2cc3sc(NC4CC5(CC(O)C5)C4)nc3n2)c(O)c1] | 371.3 |

TABLE 1-4

| | | | |
|---|---|---|---|
| 31 | 2-{5-[(cis-3-hydroxy-3-methylcyclobutyl)amino]-2H- pyrazolo[3,4-d][1,3]thiazol-2-yl}-3,5-dimethylphenyl | 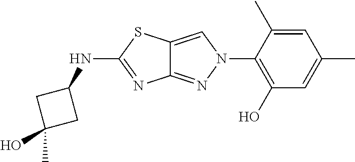 [SMILES: Cc1cc(C)c(-n2cc3sc(N[C@H]4C[C@@](C)(O)C4)nc3n2)c(O)c1] | 345.2 |
| 32 | 2-{5-[(trans-3-hydroxy-3-methylcyclobutyl)amino]-2H- pyrazolo[3,4-d][1,3]thiazol-2-yl}-3,5-dimethylphenol | 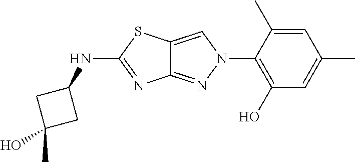 [SMILES: Cc1cc(C)c(-n2cc3sc(N[C@H]4C[C@](C)(O)C4)nc3n2)c(O)c1] | 345.2 |
| 33 | 2-(5-{[(3R,5S)-5-(difluoromethyl)-1-methylpiperidin-3- yl]amino}-2H-pyrazolo[3,4-d][1,3]thiazol-2-yl)-3,5- dimethylphenol | 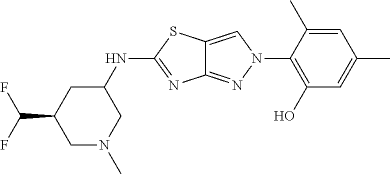 [SMILES: Cc1cc(C)c(-n2cc3sc(NC4C[C@H](C(F)F)CN(C)C4)nc3n2)c(O)c1] | 408.3 |
| 34 | 2-(5-{[(2R)-2-hydroxypropyl]amino}-2H-pyrazolo [3,4-d][1,3]thiazol-2-yl)-3,5-dimethylphenol | 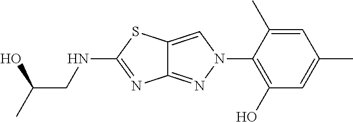 [SMILES: Cc1cc(C)c(-n2cc3sc(NC[C@@H](C)O)nc3n2)c(O)c1] | 319.1 |
| 35 | 2-(5-{[(3R)-1-(2,2-difluoroethyl)piperidin-3- yl]amino}-2H- pyrazolo[3,4-d][1,3]thiazol-2-yl)-3,5-dimethylphenol | 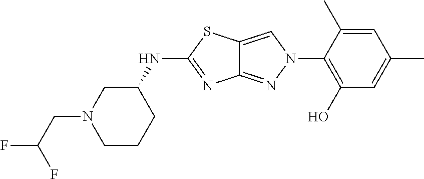 [SMILES: Cc1cc(C)c(-n2cc3sc(N[C@@H]4CCCN(CC(F)F)C4)nc3n2)c(O)c1] | 408.3 |
| 36 | 2-(5-{[(3R)-1-(2-methoxyethyl)piperidin- 3-yl]amino}-2H- pyrazolo[3,4-d][1,3]thiazol-2-yl)-3,5-dimethylphenol | 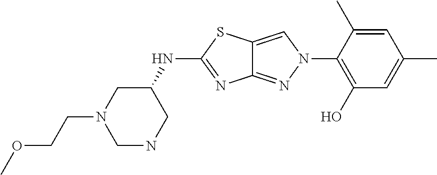 | 402.3 |
| 37 | 2-(5-{(2-methoxyethyl)[(3R)-piperidin- 3-yl]amino}-2H- pyrazolo[3,4-d][1,3]thiazol-2-yl)-3,5-dimethylphenol | 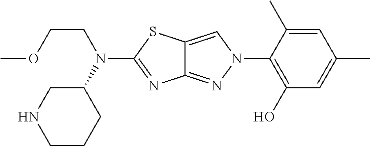 | 402.3 |
| 38 | N-(2-{[2-(2-hydroxy-4,6-dimethylphenyl)-2H- pyrazolo[3,4-d][1,3]thiazol-5-yl]amino} ethyl)acetamide | 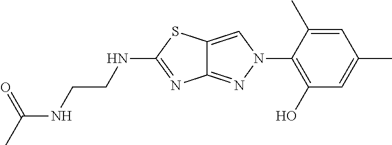 [SMILES: CC(=O)NCCNc1nc2nn(-c3c(C)cc(C)cc3O)cc2s1] | 346.2 |
| 39 | N-(4-{[2-(2-hydroxy-4,6-dimethylphenyl)-2H- pyrazolo[3,4-d][1,3]thiazol-5-yl]amino}acetamide | 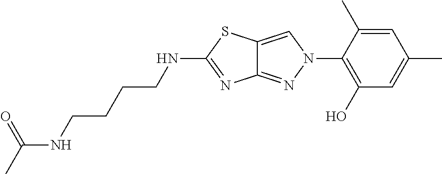 [SMILES: CC(=O)NCCCCNc1nc2nn(-c3c(C)cc(C)cc3O)cc2s1] | 374.2 |
| 40 | 2-(5-{[(3S,4S)-4-methoxy-1-methylpyrrolidin-3- yl]amino}- 2H-pyrazolo[3,4-d][1,3]thiazol-2-yl)- 3,5-dimethylphenol | 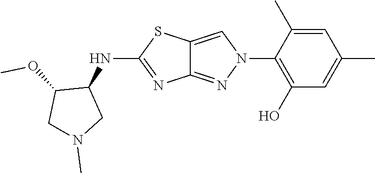 | 374.3 |

TABLE 1-5

| | | | |
|---|---|---|---|
| 41 | 2-(5-{[(3R,5R)-5-(difluoromethyl)-1-methylpiperidin-3- yl]amino}-2H-pyrazolo[3,4-d][1,3]thiazol-2-yl)-3,5- dimethylphenol | 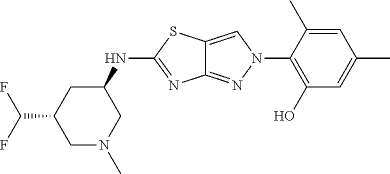 [SMILES: Cc1cc(C)c(-n2cc3sc(N[C@@H]4C[C@@H](C(F)F)CN(C)C4)nc3n2)c(O)c1] | 408.2 |
| 42 | 2-(5-{[(3R)-1-(2-fluoroethyl)piperidin-3-yl]amino}-2H- pyrazolo[3,4-d][1,3]thiazol-2-yl)-3,5-dimethylphenol | 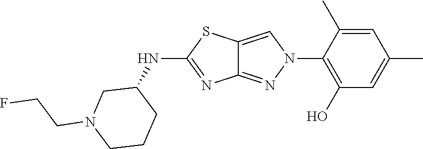 [SMILES: Cc1cc(C)c(-n2cc3sc(N[C@@H]4CCCN(CCF)C4)nc3n2)c(O)c1] | 390.3 |
| 43 | 2-(5-{[(3R,5R)-5-fluoro-1-methylpiperidin-3-yl]amino}-2H- pyrazolo[3,4-d][1,3]thiazol-2-yl)-5-methoxy-3-methylphenol | 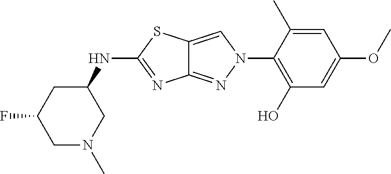 [SMILES: COc1cc(C)c(-n2cc3sc(N[C@@H]4C[C@@H](F)CN(C)C4)nc3n2)c(O)c1] | 392.2 |
| 44 | 2-(5-{[(1R,3R)-3-hydroxycyclopentyl]amino}-2H- pyrazolo[3,4-d][1,3]thiazol-2-yl)-3,5-dimethyphenol or 2-(5- {[(1S,3S)-3-hydroxycyclopentyl]amino}-2H- pyrazolo[3,4-d][1,3]thiazol-2-yl)-3,5-dimethylphenol (tR1) | 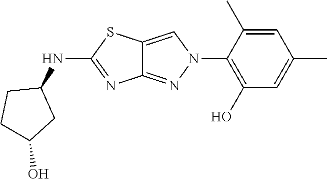 [SMILES: Cc1cc(C)c(-n2cc3sc(N[C@@H]4CC[C@@H](O)C4)nc3n2)c(O)c1] | 345.1 |
| 45 | 2-(5-{[(1R,3R)-3-hydroxycyclopentyl]amino}-2H- pyrazolo[3,4-d][1,3]thiazol-2-yl)-3,5-dimethylphenol or 2-(5- {[(1S,3S)-3-hydroxycyclopentyl]amino}-2H- pyrazolo[3,4-d][1,3]thiazol-2-yl)-3,5-dimethylphenol (tR2) | 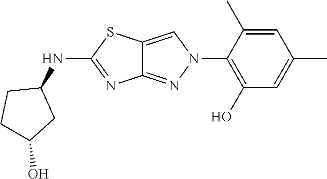 [SMILES: Cc1cc(C)c(-n2cc3sc(N[C@@H]4CC[C@@H](O)C4)nc3n2)c(O)c1] | 345.2 |
| 46 | 2-(5-{[(1R,3S)-3-hydroxycyclopentyl]amino}-2H- pyrazolo[3,4-d][1,3]thiazol-2-yl)-3,5-dimethylphenol or 2-(5- {[(1S,3R)-3-hydroxycyclopentyl]amino}-2H- pyrazolo[3,4-d][1,3]thiazol-2-yl)-3,5-dimethylphenyl (tR1) | 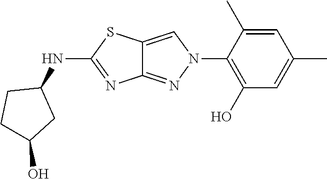 [SMILES: Cc1cc(C)c(-n2cc3sc(N[C@@H]4CC[C@H](O)C4)nc3n2)c(O)c1] | 345.1 |
| 47 | 2-(5-{[(1R,3S)-3-hydroxycyclopentyl]amino}-2H- pyrazolo[3,4-d][1,3]thiazol-2-yl)-3,5-dimethylphenol or 2-(5- {[(1S,3R)-3-hydroxycyclopentyl]amino}-2H-pyrazolo[3,4- d][1,3]thiazol-2-yl)-3,5-dimethylphenol (tR2) | 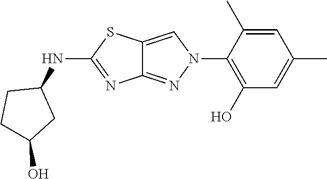 [SMILES: Cc1cc(C)c(-n2cc3sc(N[C@@H]4CC[C@H](O)C4)nc3n2)c(O)c1] | 345.2 |
| 48 | 2-(5-{[(3R,5R)-5-fluoro-1-methylpiperidin-3-yl]amino}-2H- pyrazolo[3,4-d][1,3]thiazol-2-yl)-3-methoxy-5-methylphenol | 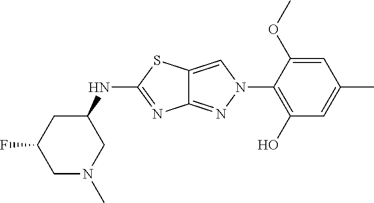 [SMILES: COc1cc(C)cc(O)c1-n1cc2sc(N[C@@H]3C[C@@H](F)CN(C)C3)nc2n1] | 392.3 |
| 49 | 2-[5-(dimethylamino)-2H-pyrazolo[3,4-d][1,3]thiazol-2-yl]- 3,5-dimethylphenol | 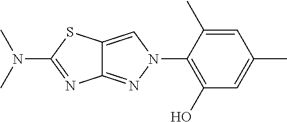 | 289.2 |
| 50 | 3-(difluoromethoxy)-2-(5-{[(3R,5R)-5-fluoro-1- methylpiperidin-3-yl]amino}-2H-pyrazolo[3,4-d][1,3]thiazol- 2-yl)-5-methylphenol | 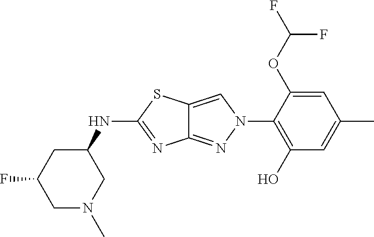 [SMILES: Cc1cc(O)c(-n2cc3sc(N[C@@H]4C[C@@H](F)CN(C)C4)nc3n2)c(OC(F)F)c1] | 428.2 |

TABLE 1-6

| | | | |
|---|---|---|---|
| 51 | 3-(difluoromethoxy)-2-(5-{[(3R,5R)-1-ethyl-5- fluoropiperidin-3-yl]amino}-2H- pyrazolo[3,4-d][1,3]thiazol-2-yl)-5-methylphenol | 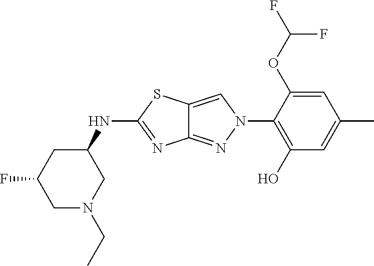 [SMILES: CCN1C[C@H](F)C[C@@H](Nc2nc3nn(-c4c(O)cc(C)cc4OC(F)F)cc3s2)C1] | 442.2 |
| 52 | 2-(5-{(2-methoxyethyl)[(3R)-1-methylpiperidin- 3-yl]amino}- 2H-pyrazolo[3,4-d][1,3]thiazol-2-yl)-3,5- dimethylphenol | 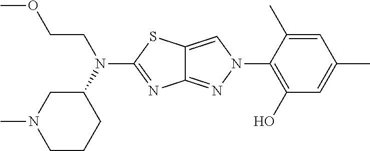 | 416.3 |
| 53 | 2-(5-{[2-(dimethylamino)ethyl]amino}-2H- pyrazolo[3,4-d][1,3]thiazol-2-yl)-3,5-dimethylphenol | 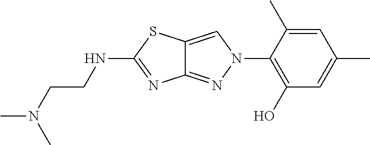 | 332.2 |
| 54 | 3,5-dimethyl-2-(5-{[2-(morpholin-4-yl) ethyl]amino}-2H- pyrazolo[3,4-d][1,3]thiazol-2-yl)phenol | 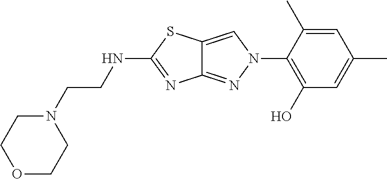 [SMILES: Cc1cc(C)c(-n2cc3sc(NCCN4CCOCC4)nc3n2)c(O)c1] | 374.3 |
| 55 | 2-{5-[(3-hydroxy-3-methylbutyl)amino]-2H- pyrazolo[3,4-d][1,3]thiazol-2-yl}-3,5-dimethylphenol | 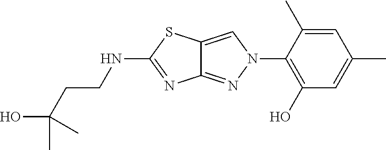 [SMILES: Cc1cc(C)c(-n2cc3sc(NCCC(C)(C)O)nc3n2)c(O)c1] | 347.2 |
| 56 | N-(3-{[2-(2-hydroxy-4,6-dimethylphenyl)-2H- pyrazolo[3,4-d][1,3]thiazol-5-yl]amino}propyl) acetamide | 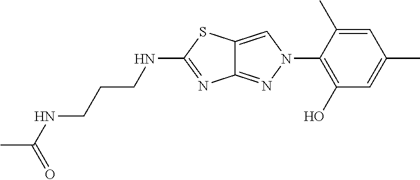 [SMILES: CC(=O)NCCCNc1nc2nn(-c3c(C)cc(C)cc3O)cc2s1] | 360.2 |
| 57 | 4-{[2-(2-hydroxy-4,6-dimethylphenyl)-2H-pyrazolo[3,4- d][1,3]thiazol-5-yl]amino}bicyclo[2.2.1]heptan-1-ol | 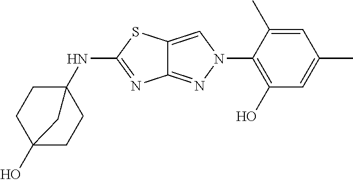 [SMILES: Cc1cc(C)c(-n2cc3sc(NC45CCC(O)(CC4)C5)nc3n2)c(O)c1] | 371.3 |
| 58 | 5-methyl-2-(3-methyl-5-{[(3R)-1-methylpiperidin-3- yl]amino}-2H-pyrazolo[3,4-d][1,3]thiazol-2-yl)pyridin- 3-ol | 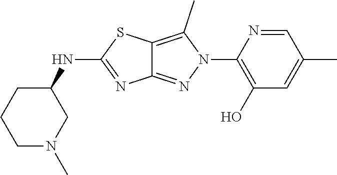 [SMILES: Cc1cnc(-n2nc3nc(N[C@@H]4CCCN(C)C4)sc3c2C)c(O)c1] | 359.2 |
| 59 | 3,5-diethyl-2-(5-{[(3R,5R)-5-fluoro-1-methylpiperidin-3- yl]amino}-2H-pyrazolo[3,4-d][1,3]thiazol-2-yl)phenol | 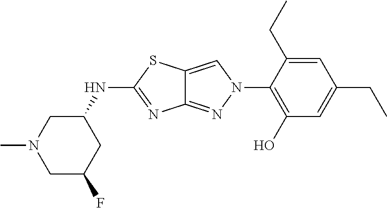 [SMILES: CCc1cc(O)c(-n2cc3sc(N[C@@H]4C[C@@H](F)CN(C)C4)nc3n2)c(CC)c1] | 404.3 |
| 60 | 5-ethyl-2-(5-{[(3R,5R)-5-fluoro-1-methylpiperidin-3- yl]amino}-2H-pyrazolo[3,4-d][1,3]thiazol-2-yl)-3- methylphenol | 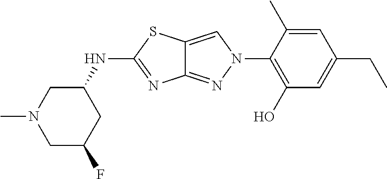 [SMILES: CCc1cc(C)c(-n2cc3sc(N[C@@H]4C[C@@H](F)CN(C)C4)nc3n2)c(O)c1] | 390.2 |

TABLE 1-7

| | | | |
|---|---|---|---|
| 61 | 2-{5-[(2-methoxyethyl)(methyl)amino]-2H- pyrazolo[3,4-d][1,3]thiazol-2-yl}-3,5-dimethylphenol | 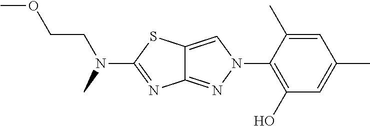 | 333.1 |
| 62 | 2-(5-{[(3R,5R)-5-fluoropiperidin-3-yl]amino}-2H- pyrazolo[3,4-d][1,3]thiazol-2-yl)-3,5-dimethylphenol | 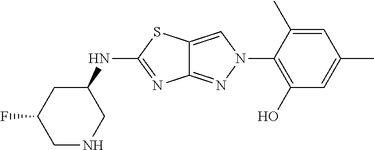 [SMILES: Cc1cc(C)c(-n2cc3sc(N[C@H]4CNC[C@H](F)C4)nc3n2)c(O)c1] | 362.2 |
| 63 | 5-methyl-2-(5-{[(3R)-1-methylpiperidin-3-yl]amino}-2H- pyrazolo[3,4-d][1,3]thiazol-2-yl)pyridin-3-ol | 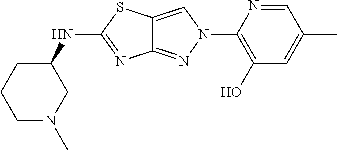 [SMILES: Cc1cnc(-n2cc3sc(N[C@@H]4CCCN(C)C4)nc3n2)c(O)c1] | 345.2 |
| 64 | 3-cyclopropyl-2-(5-{[(2R)-2-hydroxypropyl]amino}-2H- pyrazolo[3,4-d][1,3]thiazol-2-yl)-5-methylphenol | 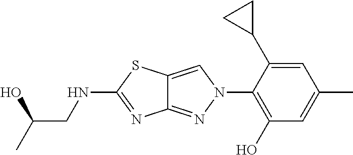 [SMILES: Cc1cc(O)c(-n2cc3sc(NC[C@@H](C)O)nc3n2)c(C2CC2)c1] | 345.1 |
| 65 | 3-cyclopropyl-2-(5-{[(3R,5R)-5-fluoro-1-methylpiperidin-3- yl]amino}-2H-pyrazolo[3,4-d][1,3]thiazol-2-yl)-5- methylphenol | 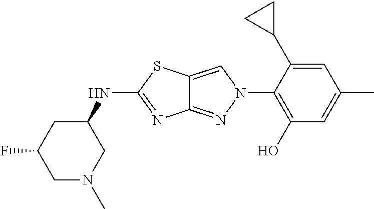 [SMILES: Cc1cc(O)c(-n2cc3sc(N[C@@H]4C[C@@H](F)CN(C)C4)nc3n2)c(C2CC2)c1] | 402.2 |
| 66 | 2-{5-[(2-hydroxy-2-methylpropyl)amino]-2H- pyrazolo[3,4-d][1,3]thiazol-2-yl}-3,5-dimethylphenol | 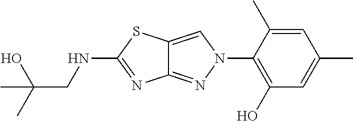 [SMILES: Cc1cc(C)c(-n2cc3sc(NCC(C)(C)O)nc3n2)c(O)c1] | 333.2 |
| 67 | 2-(5-{[1-(2,2-difluoroethyl)pyrrolidin-3-yl]amino}-2H- pyrazolo[3,4-d][1,3]thiazol-2-yl)-3,5-dimethylphenol | 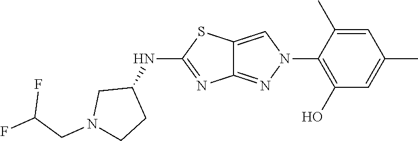 [SMILES: Cc1cc(C)c(-n2cc3sc(N[C@@H]4CCN(CC(F)F)C4)nc3n2)c(O)c1] | 394.3 |
| 68 | 3,3-difluoro-1-(3-{[2-(2-hydroxy-4,6-dimethylphenyl)-2H- pyrazolo[3,4-d][1,3]thiazol-5-yl]amino}pyrrolidin-1- yl)propan-1-one | 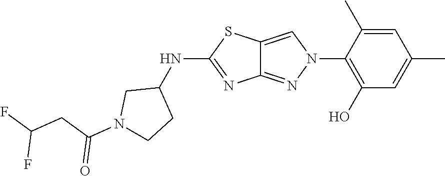 [SMILES: Cc1cc(C)c(-n2cc3sc(NC4CCN(C(=O)CC(F)F)C4)nc3n2)c(O)c1] | 422.2 |
| 69 | 2,2,2-trichloroethyl 3-{[2-(2-hydroxy-4,6-dimethylphenyl)- 2H-pyrazolo[3,4-d][1,3]thiazol-5-yl]amino}pyrrolidine-1- carboxylate | 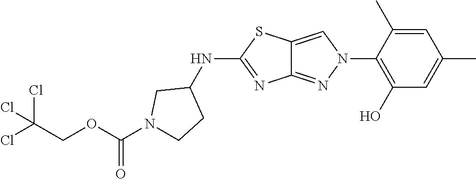 [SMILES: Cc1cc(C)c(-n2cc3sc(NC4CCN(C(=O)OCC(Cl)(Cl)Cl)C4)nc3n2)c(O)c1] | 504.1 |
| 70 | 2-(5-{[(3R,5R)-5-fluoro-1-methylpiperidin-3-yl]amino}-3- methyl-2H-pyrazolo[3,4-d][1,3]thiazol-2-yl)-3,5- dimethylphenol | 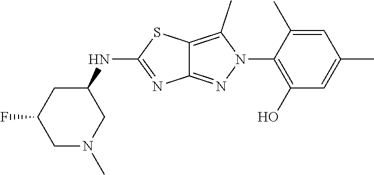 [SMILES: Cc1cc(C)c(-n2nc3nc(N[C@@H]4C[C@@H](F)CN(C)C4)sc3c2C)c(O)c1] | 390.1 |

TABLE 1-8

| | | | |
|---|---|---|---|
| 71 | 3,5-dimethyl-2-[5-({(3R)-1-[(S)-oxolan-3-yl]piperidin- 3- yl}amino)-2H-pyrazolo[3,4-d][1,3]thiazol-2-yl]phenol or 3,5-dimethyl-2-[5-({(3R)-1-[(R)-oxolan-3-yl]piperidin- 3-yl}amino)-2H-pyrazolo[3,4-d][1,3]thiazol-2-yl]phenol (tR1) | 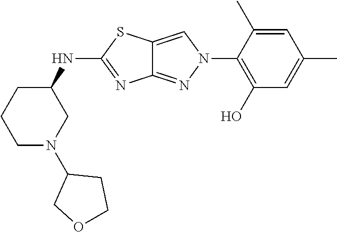 [SMILES: Cc1cc(C)c(-n2cc3sc(N[C@@H]4CCCN(C5CCOC5)C4)nc3n2)c(O)c1] | 414.2 |
| 72 | 3,5-dimethyl-2-[5-({(3R)-1-[(S)-oxolan-3-yl]piperidin-3- yl}amino)-2H-pyrazolo[3,4-d][1,3]thiazol-2-yl]phenol or 3,5-dimethyl-2-[5-({(3R)-1-[(R)-oxolan-3-yl]piperidin-3- yl}amino)-2H-pyrazolo[3,4-d][1,3]thiazol-2-yl]phenol (tR2) | 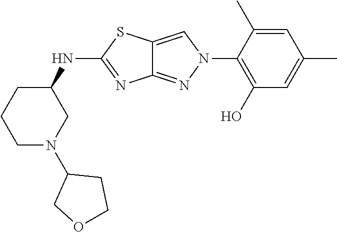 [SMILES: Cc1cc(C)c(-n2cc3sc(N[C@@H]4CCCN(C5CCOC5)C4)nc3n2)c(O)c1] | 414.2 |
| 73 | 2-[5-({(3R,5R)-5-fluoro-1-[(S)-oxolan-3-yl]piperidin-3- yl}amino)-2H-pyrazolo[3,4-d][1,3]thiazol-2-yl]-3,5- dimethylphenol or 2-[5-({(3R,5R)-5-fluoro- 1-[(R)-oxolan-3- yl]piperidin-3-yl}amino)-2H-pyrazolo[3,4-d][1,3]thiazol- 2-yl]-3,5-dimethylphenol (tR1) | 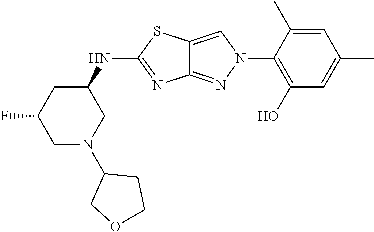 [SMILES: Cc1cc(C)c(-n2cc3sc(N[C@@H]4C[C@@H](F)CN(C5CCOC5)C4)nc3n2)c(O)c1] | 432.2 |
| 74 | 2-[5-({(3R,5R)-5-fluoro-1-[(S)-oxolan-3-yl]piperidin-3- yl}amino)-2H-pyrazolo[3,4-d][1,3]thiazol-2-yl]-3,5- dimethylphenol or 2-[5-({(3R,5R)-5- fluoro-1-[(R)-oxolan-3- yl]piperidin-3-yl}amino)-2H-pyrazolo[3,4-d][1,3]thiazol- 2-yl]-3,5-dimethylphenol (tR2) | 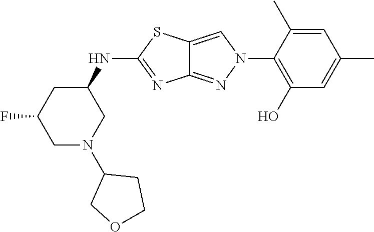 [SMILES: Cc1cc(C)c(-n2cc3sc(N[C@@H]4C[C@@H](F)CN(C5CCOC5)C4)nc3n2)c(O)c1] | 432.2 |
| 75 | 5-chloro-3-(difluoromethyl)-2-(5-{[(3R,5R)-5-fluoro-1- methylpiperidin-3-yl]amino}-2H-pyrazolo [3,4-d][1,3]thiazol-2-yl)phenol | 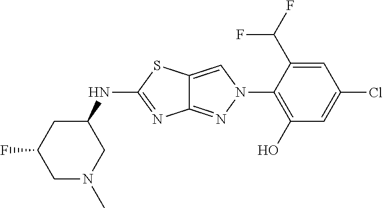 [SMILES: CN1C[C@H](F)C[C@@H](Nc2nc3nn(-c4c(O)cc(Cl)cc4C(F)F)cc3s2)C1] | 432.1 |
| 76 | 3-(5-{[(3R,5R)-5-methoxy-1-methylpiperidin-3- yl]amino}-2H-pyrazolo[3,4-d][1,3]thiazol-2-yl)-3,5- dimethylphenol | 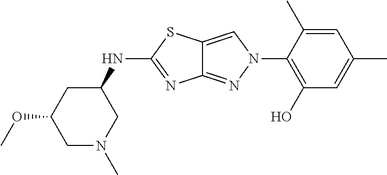 | 388.1 |
| 77 | benzyl 3-{[2-(2-hydroxy-4,6-dimethylphenyl)-2H- pyrazolo[3,4-d][1,3]thiazol-5-yl]amino}pyrrolidine-1- carboxylate | 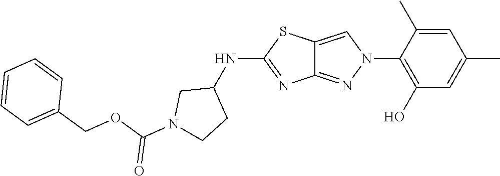 [SMILES: Cc1cc(C)c(-n2cc3sc(NC4CCN(C(=O)OCc5ccccc5)C4)nc3n2)c(O)c1] | 464.2 |
| 78 | 5-chloro-3-(difluoromethyl)-2-(5-{[(3R,5R)-1-ethyl-5- fluoropiperidin-3-yl]amino}-2H- pyrazolo[3,4-d][1,3]thiazol-2-yl)phenol | 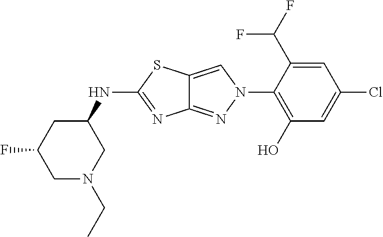 [SMILES: CCN1C[C@H](F)C[C@@H](Nc2nc3nn(-c4c(O)cc(Cl)cc4C(F)F)cc3s2)C1] | 446.1 |
| 79 | 2-(5-{[(3R,4R)-4-fluoro-1- methylpyrrolidin-3-yl]amino}-2H- pyrazolo[3,4-d][1,3]thiazol-2-yl)-3,5-dimethylphenol | 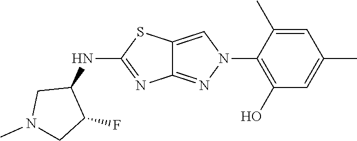 | 362.1 |
| 80 | 2-(5-{[(3S,4S)-4-fluoro-1- methylpyrrolidin-3-yl]amino}-2H- pyrazolo[3,4-d][1,3]thiazol-2-yl)-3,5-dimethylphenol | 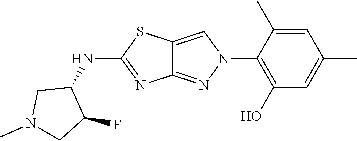 | 362.1 |

TABLE 1-9

| | | | |
|---|---|---|---|
| 81 | 3,5-dimethyl-2-{5-[(1-methylpiperidin-4-yl)amino]-2H- pyrazolo[3,4-d][1,3]thiazol-2-yl}phenol | 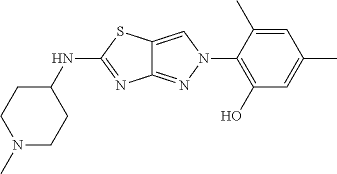 [SMILES: Cc1cc(C)c(-n2cc3sc(NC4CCN(C)CC4)nc3n2)c(O)c1] | 358.2 |
| 82 | 5-chloro-2-[5-{[(3R,5R)-5-fluoro-1-methylpiperidin-3- yl]amino}-4-methylimidazo[4,5-c]pyrazol-2(4H)-yl]-3- methylphenol | 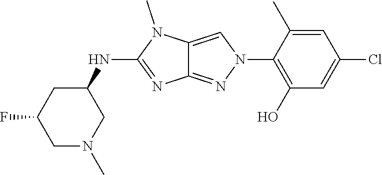 [SMILES: Cc1cc(Cl)cc(O)c1-n1cc2c(nc(N[C@@H]3C[C@@H](F)CN(C)C3)n2C)n1] | 393.1 |
| 83 | 2-{5-[(2-hydroxybutyl)amino]-2H- pyrazolo[3,4-d][1,3]thiazol-2-yl}-3,5-dimethylphenol | 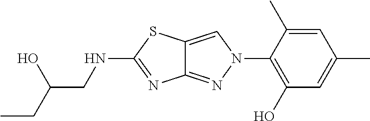 [SMILES: CCC(O)CNc1nc2nn(-c3c(C)cc(C)cc3O)cc2s1] | 333.2 |
| 84 | 2-(5-{[(1-hydroxycyclopropyl)methyl]amino}-2H- pyrazolo[3,4-d][1,3]thiazol-2-yl)-3,5-dimethylphenol | 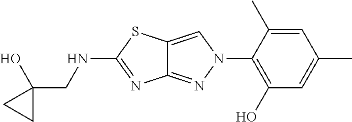 [SMILES: Cc1cc(C)c(-n2cc3sc(NCC4(O)CC4)nc3n2)c(O)c1] | 331.1 |
| 85 | 1-(3-{[2-(2-hydroxy-4,6-dimethylphenyl)-2H- pyrazolo[3,4-d][1,3]thiazol-5-yl]amino}pyrrolidin-1- yl)ethan-1-one | 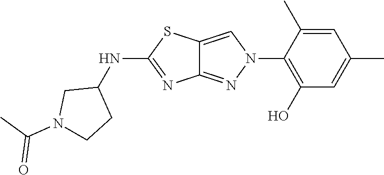 [SMILES: CC(=O)N1CCC(Nc2nc3nn(-c4c(C)cc(C)cc4O)cc3s2)C1] | 372.2 |
| 86 | 3-{[2-(2-hydroxy-4,6-dimethylphenyl)-2H- pyrazolo[3,4-d][1,3]thiazol-5-yl]amino}-N- methylpyrrolidine-1-carboxamide | 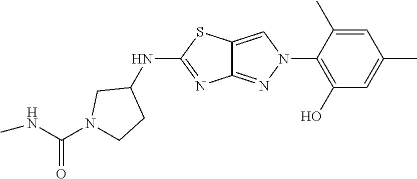 | 387.2 |
| 87 | 3-ethyl-2-(5-{[(2R)-2-hydroxypropyl]amino}-2H- pyrazolo[3,4-d][1,3]thiazol-2-yl)-5-methylphenol | 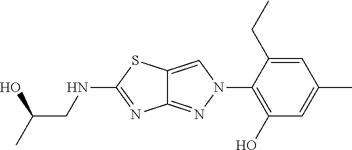 [SMILES: CCc1cc(C)cc(O)c1-n1cc2sc(NC[C@@H](C)O)nc2n1] | 333.2 |
| 88 | 1-(3-{[2-(2-hdyroxy-4,6-dimethylphenyl)-2H- pyrazolo[3,4-d][1,3]thiazol-5-yl]amino}pyrrolidin-1- yl)propan-1-one | 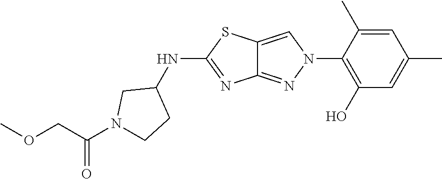 | 386.3 |
| 89 | 1-(3-{[2-(2-hydroxy-4,6-dimethylphenyl)-2H- pyrazolo[3,4-d][1,3]thiazol-5-yl]amino}pyrrolidin-1-yl)- 2-methoxyethan-1-one | 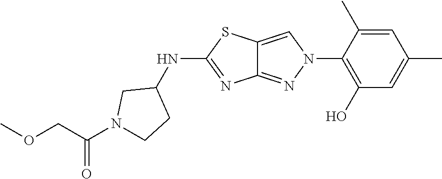 | 402.3 |
| 90 | 2,2-difluoro-1-(3-{[2-(2-hydroxy-4,6-dimethylphenyl)- 2H-pyrazolo[3,4-d][1,3]thiazol-5-yl]amino}pyrrolidin- 1-yl)ethan-1-one | 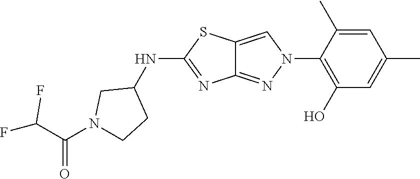 [SMILES: Cc1cc(C)c(-n2cc3sc(NC4CCN(C(=O)C(F)F)C4)nc3n2)c(O)c1] | 408.2 |

TABLE 1-10

| | | | |
|---|---|---|---|
| 91 | methyl 3-{[2-(2-hydroxy-4,6-dimethylphenyl)- 2H-pyrazolo[3,4-d][1,3]thiazol-5-yl]amino}pyrrolidine- 1-carboxylate | 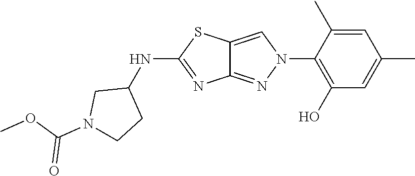 | 388.3 |
| 92 | 3,5-dimethyl-2-{5-[(1-methylpyrrolidin-3-yl)amino]- 2H-pyrazolo[3,4-d][1,3]thiazol-2-yl}phenol | 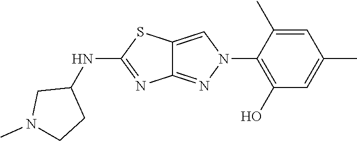 | 344.2 |
| 93 | 2-(5-{[(2R)-2-hydroxypropyl]amino}- 2H-pyrazolo[3,4-d][1,3]thiazol-2-yl)5-methyl-3- (propan-2-yl)phenol | 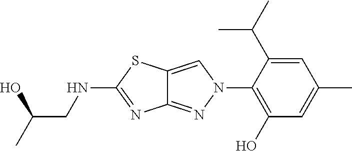 [SMILES: Cc1cc(O)c(-n2cc3sc(NC[C@@H](C)O)nc3n2)c(C(C)C)c1] | 347.2 |
| 94 | 2-(5-{[(3R,5R)-5-fluoro-1-methylpiperidin-3-yl]amino}- 2H-pyrazolo[3,4-d][1,3]thiazol-2-yl)-5-methyl-3- (propan-2-yl)phenol | 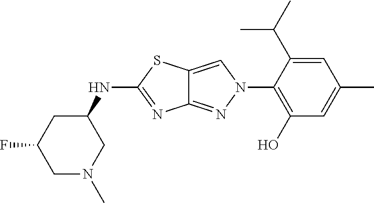 [SMILES: Cc1cc(O)c(-n2cc3sc(N[C@@H]4C[C@@H](F)CN(C)C4)nc3n2)c(C(C)C)c1] | 404.2 |
| 95 | 4-{[2-(2-hydroxy-4,6-dimethylphenyl)- 2H-pyrazolo[3,4-d][1,3]thiazol-5-yl]amino}-1- methylpyrrolidin-2-one | 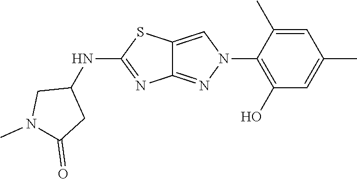 | 358.2 |
| 96 | cyclopropyl(3-{[2-(2-hydroxy-4,6-dimethylphenyl)- 2H-pyrazolo[3,4-d][1,3]thiazol-5-yl]amino} pyrrolidin-1-yl)methanone | 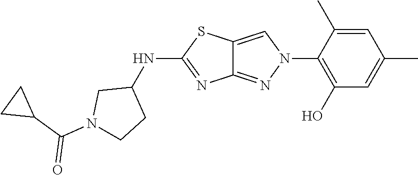 [SMILES: Cc1cc(C)c(-n2cc3sc(NC4CCN(C(=O)C5CC5)C4)nc3n2)c(O)c1] | 398.3 |
| 97 | 2-cyclopropyl-1-(3-{[2-(2-hydroxy-4,6-dimethylphenyl)- 2H-pyrazolo[3,4-d][1,3]thiazol-5-yl]amino} pyrrolidin-1-yl)ethan-1-one | 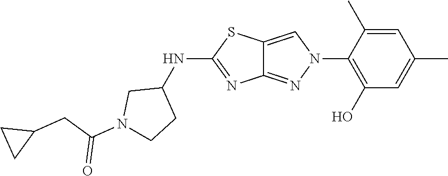 [SMILES: Cc1cc(C)c(-n2cc3sc(NC4CCN(C(=O)CC5CC5)C4)nc3n2)c(O)c1] | 412.3 |
| 98 | (1-fluorocyclopropyl)(3-{[2-(2-hydroxy-4,6- dimethylphenyl)-2H-pyrazolo[3,4-d][1,3]thiazol-5-yl] amino}pyrrolidin-1-yl)methanone | 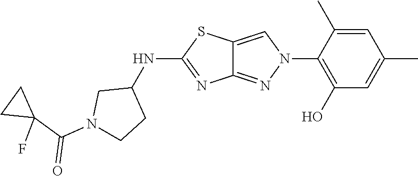 [SMILES: Cc1cc(C)c(-n2cc3sc(NC4CCN(C(=O)C5(F)CC5)C4)nc3n2)c(O)c1] | 416.2 |
| 99 | 1-(3-{[2-(2-hydroxy-4,6-dimethylphenyl)- 2H-pyrazolo[3,4-d][1,3]thiazol-5-yl]amino} pyrrolidine-1-carbonyl)cyclopropane-1-carbonitrile | 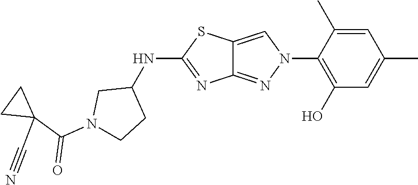 [SMILES: Cc1cc(C)c(-n2cc3sc(NC4CCN(C(=O)C5(C#N)CC5)C4)nc3n2)c(O)c1] | 423.2 |
| 100 | 2-(5-{[1-(methanesulfonyl)pyrrolidin-3-yl]amino}- 2H-pyrazolo[3,4-d][1,3]thiazol-2-yl)-3,5- dimethylphenol | 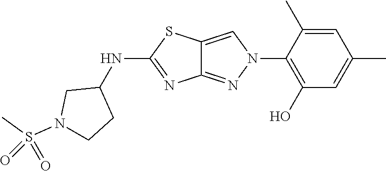 | 408.2 |

TABLE 1-11

| | | | |
|---|---|---|---|
| 101 | 2-(5-{[1-(cyclopropanesulfonyl)pyrrolidin-3-yl]amino}- 2H-pyrazolo[3,4-d][1,3]thiazol-2-yl)-3,5-dimethylphenol | 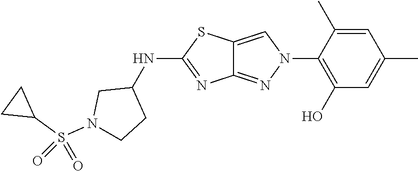 [SMILES: Cc1cc(C)c(-n2cc3sc(NC4CCN(S(=O)(=O)C5CC5)C4)nc3n2)c(O)c1] | 434.2 |
| 102 | 2-hydroxy-1-(3-{[2-(2-hydroxy-4,6-dimethylphenyl)- 2H-pyrazolo[3,4-d][1,3]thiazol-5-yl]amino}pyrrolidin-1-yl)- 2-methylpropan-1-one | 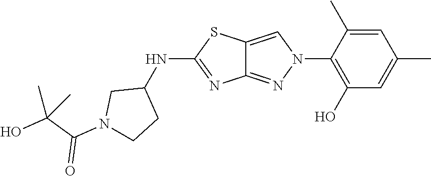 [SMILES: Cc1cc(C)c(-n2cc3sc(NC4CCN(C(=O)C(C)(C)O)C4)nc3n2)c(O)c1] | 416.2 |
| 103 | 3-{[2-(2-hydroxy-4,6-dimethylphenyl)- 2H-pyrazolo[3,4-d][1,3]thiazol-5-yl]amino}-1- methylpyrrolidin-2-one | 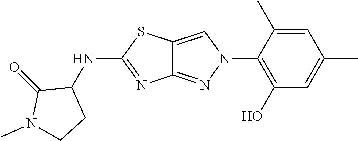 | 358.1 |
| 104 | racemic mixture of 1-[(3R,4R)-3-fluoro-4-{[2-(2-hydroxy- 4,6-dimethylphenyl)-2H-pyrazolo[3,4-d][1,3]thiazol- 5-yl]amino}pyrrolidin-1-yl]ethan-1-one and 1-[(3S,4S)-3- fluoro-4-{[2-(2-hydroxy-4,6-dimethylphenyl)- 2H-pyrazolo[3,4-d][1,3]thiazol-5-yl]amino}pyrrolidin-1-yl] ethan-1-one | 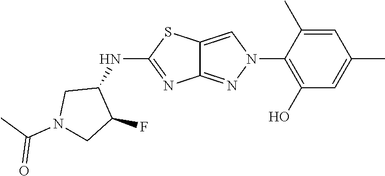 [SMILES: CC(=O)N1C[C@H](Nc2nc3nn(-c4c(C)cc(C)cc4O)cc3s2)[C@@H](F)C1] | 390.3 |
| 105 | racemic mixture of 1-[(3R,4R)-3-fluoro-4-{[2-(2-hydroxy- 4,6-dimethylphenyl)-2H-pyrazolo[3,4-d][1,3]thiazol-5- yl]amino}pyrrolidin-1-yl]propan-1-one and 1-[(3S,4S)-3- fluoro-4-{[2-(2-hydroxy-4,6-dimethylphenyl)- 2H-pyrazolo[3,4-d][1,3]thiazol-5-yl]amino}pyrrolidin-1- yl]propan-1-one | 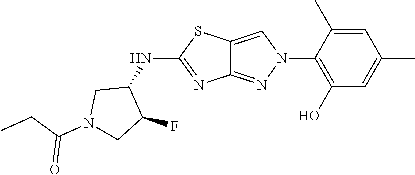 [SMILES: CCC(=O)N1C[C@H](Nc2nc3nn(-c4c(C)cc(C)cc4O)cc3s2)[C@@H](F)C1] | 404.2 |
| 106 | racemic mixture of 2-cyclopropyl-1-[(3R,4R)-3-fluoro-4- {[2-(2-hydroxy-4,6-dimethylphenyl)- 2H-pyrazolo[3,4-d][1,3]thiazol-5-yl]amino}pyrrolidin- 1-yl]ethan-1-one and 2-cyclopropyl-1-[(3S,4S)-3- fluoro-4-{[2-(2-hydroxy-4,6-dimethylphenyl)- 2H-pyrazolo[3,4-d][1,3]thiazol-5-yl]amino}pyrrolidin-1- yl]ethan-1-one | 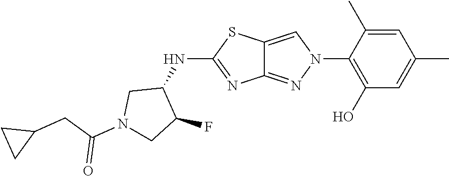 [SMILES: Cc1cc(C)c(-n2cc3sc(N[C@H]4CN(C(=O)CC5CC5)C[C@@H]4F)nc3n2)c(O)c1] | 430.2 |
| 107 | 5-(difluoromethoxy)-2-(5-{[(3R,5R)-5-fluoro-1- methylpiperidin-3-yl]amino}- 2H-pyrazolo[3,4-d][1,3]thiazol-2-yl)-3-methylphenol | 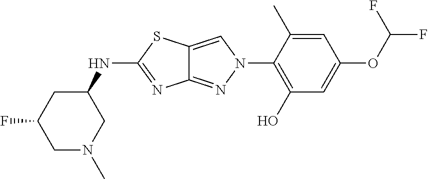 [SMILES: Cc1cc(OC(F)F)cc(O)c1-n1cc2sc(N[C@@H]3C[C@@H](F)CN(C)C3)nc2n1] | 428.2 |
| 108 | 1-(3-{[2-(2-hydroxy-4,6-dimethylphenyl)- 2H-pyrazolo[3,4-d][1,3]thiazol-5-yl]amino}piperidin-1- yl)ethan-1-one | 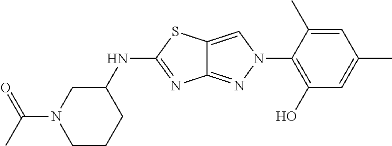 [SMILES: CC(=O)N1CCCC(Nc2nc3nn(-c4c(C)cc(C)cc4O)cc3s2)C1] | 386.3 |
| 109 | 1-(3-{[2-(2-hydroxy-4,6-dimethylphenyl)- 2H-pyrazolo[3,4-d][1,3]thiazol-5-yl]amino}piperidin-1- yl)propan-1-one | 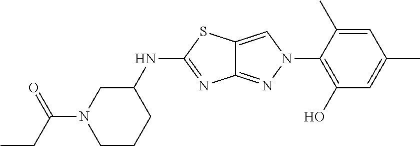 [SMILES: CCC(=O)N1CCCC(Nc2nc3nn(-c4c(C)cc(C)cc4O)cc3s2)C1] | 400.3 |
| 110 | methyl 3-{[2-(2-hydroxy-4,6-dimethylphenyl)- 2H-pyrazolo[3,4-d][1,3]thiazol-5-yl]amino}piperidine-1- carboxylate | 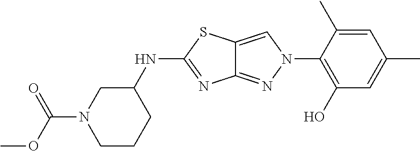 | 402.3 |

TABLE 1-12

| | | | |
|---|---|---|---|
| 111 | 3-{[2-(2-hydroxy-4,6-dimethylphenyl) 2H-pyrazolo[3,4-d][1,3]thiazol-5-yl]amino}-N-methyl- piperidine-1-carboxamide | 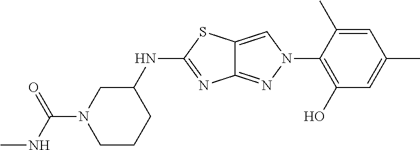 | 401.3 |
| 112 | 2-(5-{[1-(methanesulfonyl)piperidin-3-yl]amino}- 2H-pyrazolo[3,4-d][1,3]thiazol-2-yl)-3,5-dimethylphenol | 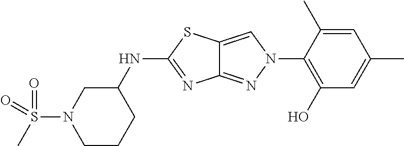 [SMILES: Cc1cc(C)c(-n2cc3sc(NC4CCCN(S(C)(=O)=O)C4)nc3n2)c(O)c1] | 422.2 |
| 113 | 3,3-difluoro-1-(3-{[2-(2-hydroxy-4,6-dimethylphenyl)- 2H-pyrazolo[3,4-d][1,3]thiazol-5-yl]amino}piperidin-1- yl)propan-1-one | 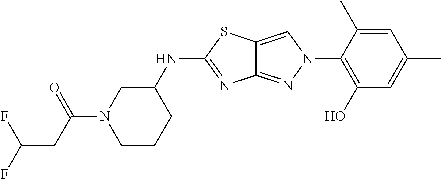 [SMILES: Cc1cc(C)c(-n2cc3sc(NC4CCCN(C(=O)CC(F)F)C4)nc3n2)c(O)c1] | 436.2 |
| 114 | 2-{5-[(2-methoxy-2-methylpropyl)amino]- 2H-pyrazolo[3,4-d][1,3]thiazol-2-yl}-3,5-dimethylphenol | 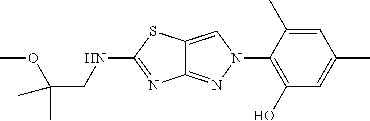 | 347.2 |
| 115 | 5-{[2-(2-hydroxy-4,6-dimethylphenyl)- 2H-pyrazolo[3,4-d][1,3]thiazol-5-yl]amino}-1- methylpiperidin-2-one | 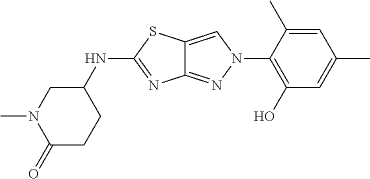 | 372.2 |
| 116 | 3,5-dimethyl-2-[5-({[(2S)-oxolan-2-yl]methyl}amino)- 2H-pyrazolo[3,4-d][1,3]thiazol-2-yl]phenol | 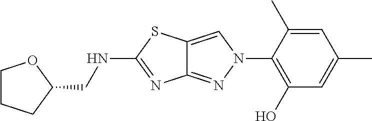 [SMILES: Cc1cc(C)c(-n2cc3sc(NC[C@@H]4CCCO4)nc3n2)c(O)c1] | 345.2 |
| 117 | 3,5-dimethyl-2-[5-({[(2R)-oxolan-2-yl]methyl}amino)- 2H-pyrazolo[3,4-d][1,3]thiazol-2-yl]phenol | 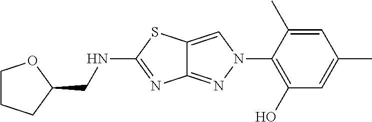 [SMILES: Cc1cc(C)c(-n2cc3sc(NC[C@H]4CCCO4)nc3n2)c(O)c1] | 345.2 |
| 118 | 3-{[2-(2-hydroxy-4,6-dimethylphenyl)- 2H-pyrazolo[3,4-d][1,3]thiazol-5-yl]amino}-1- methylpiperidin-2-one | 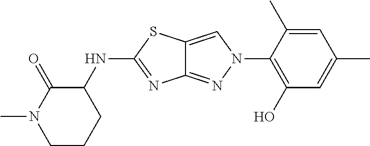 | 372.2 |
| 119 | racemic mixture of (3R,4R)-3-fluoro-4-{[2-(2-hydroxy- 4,6-dimethylphenyl)-2H-pyrazolo[3,4-d][1,3]thiazol-5- yl]amino}-N-methylpyrrolidine-1-carboxamide and (3S,4S)-3-fluoro-4-{[2-(2-hydroxy-4,6-dimethylphenyl)- 2H-pyrazolo[3,4-d][1,3]thiazol-5-yl]amino}-N- methylpyrrolidine-1-carboxamide | 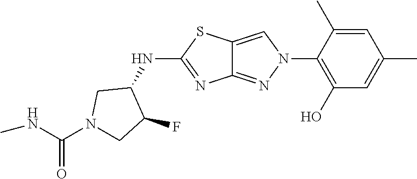 | 405.2 |
| 120 | 2-(5-{[(2R)-2-methoxypropyl]amino}- 2H-pyrazolo[3,4-d][1,3]thiazol-2-yl)-3,5-dimethylphenol | 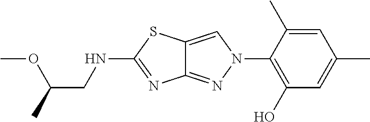 | 333.2 |

TABLE 1-13

| | | | |
|---|---|---|---|
| 121 | racemic mixture of 2-(5-{[(3R,4S)-4-fluorooxolan-3- yl]amino}-2H-pyrazolo[3,4-d][1,3]thiazol-2-yl)-3,5- dimethylphenol and 2-(5-{[(3S,4R)-4-fluorooxolan-3- yl]amino}-2H-pyrazolo[3,4-d][1,3]thiazol-2-yl)-3,5- dimethylphenol | 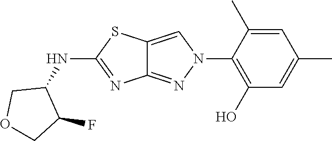 [SMILES: Cc1cc(C)c(-n2cc3sc(N[C@H]4COC[C@@H]4F)nc3n2)c(O)c1] | 349.2 |
| 122 | racemic mixture of 2-(5-{[(3R,4R)-4-fluorooxolan-3- yl]amino}-2H-pyrazolo[3,4-d][1,3]thiazol-2-yl)-3,5- dimethylphenol and 2-(5-{[(3S,4S)-4-fluorooxolan-3- yl]amino}-2H-pyrazolo[3,4-d][1,3]thiazol-2-yl)-3,5- dimethylphenol | 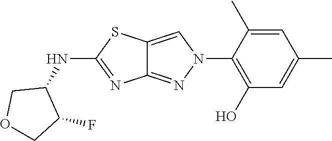 [SMILES: Cc1cc(C)c(-n2cc3sc(N[C@H]4COC[C@H]4F)nc3n2)c(O)c1] | 349.1 |
| 123 | racemic mixture of 1-[(3R,4R)-3-fluoro-4-{[2-(2-hydroxy- 4,6-dimethylphenyl)-2H-pyrazolo[3,4-d][1,3]thiazol-5- yl]amino}pyrrolidin-1-yl]-2-hydroxy-2-methylpropan-1-one and 1-[(3S,4S)-3-fluoro-4-{[2-(2-hydroxy-4,6- dimethylphenyl)-2H-pyrazolo[3,4-d][1,3]thiazol-5- yl]amino}pyrrolidin-1-yl]-2-hydroxy-2-methylpropan-1-one | 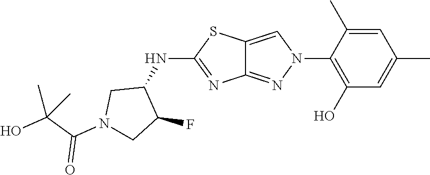 [SMILES: Cc1cc(C)c(-n2cc3sc(N[C@H]4CN(C(=O)C(C)(C)O)C[C@@H]4F)nc3n2)c(O)c1] | 434.2 |
| 124 | racemic mixture of [(3R,4R)-3-fluoro-4-{[2-(2-hydroxy-4,6- dimethylphenyl)-2H-pyrazolo[3,4-d][1,3]thiazol-5- yl]amino}pyrrolidin-1-yl](1-hydroxycyclopropyl) methanone and [(3S,4S)-3-fluoro-4-{[2-(2-hydroxy-4,6- dimethylphenyl)-2H-pyrazolo[3,4-d][1,3]thiazol-5-yl] amino}pyrrolidin-1-yl](1-hydroxycyclopropyl)methanone | 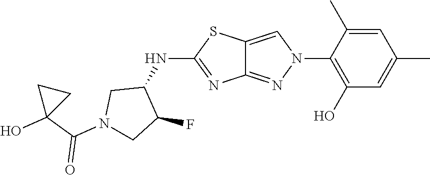 [SMILES: Cc1cc(C)c(-n2cc3sc(N[C@H]4CN(C(=O)C5(O)CC5)C[C@@H]4F)nc3n2)c(O)c1] | 432.2 |
| 125 | racemic mixture of 1-[(3R,4R)-3-fluoro-4-{[2-(2-hydroxy- 4,6-dimethylphenyl)-2H-pyrazolo[3,4-d][1,3]thiazol-5- yl]amino}pyrrolidine-1-carbonyl]cyclopropane-1- carbonitrile and 1-[(3S,4S)-3-fluoro-4-{[2-(2-hydroxy-4,6- dimethylphenyl)-2H-pyrazolo[3,4-d][1,3]thiazol-5- yl]amino}pyrrolidine-1-carbonyl]cyclopropane-1- carbonitrile | 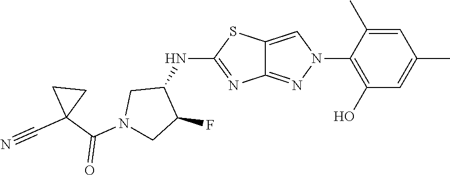 [SMILES: Cc1cc(C)c(-n2cc3sc(N[C@H]4CN(C(=O)C5(C#N)CC5)C[C@@H]4F)nc3n2)c(O)c1] | 441.2 |
| 126 | 3-[(R)-2,2-difluorocyclopropyl]-2-(5-{[(3R,5R)-5-fluoro-1- methylpiperidin-3-yl]amino}-2H-pyrazolo[3,4-d][1,3]thiazol- 2-yl)-5-methylphenol or 3-[(S)-2,2-difluorocyclopropyl]-2- (5-{[(3R,5R)-5-fluoro-1-methylpiperidin-3-yl]amino}- 2H-pyrazolo[3,4-d][1,3]thiazol-2-yl)-5-methylphenol (tR2) | 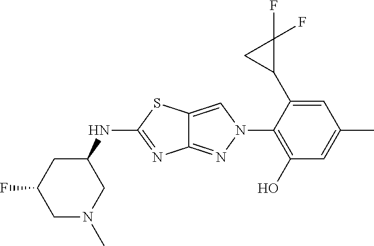 [SMILES: Cc1cc(O)c(-n2cc3sc(N[C@@H]4C[C@@H](F)CN(C)C4)nc3n2)c(C2CC2(F)F)c1] | 438.2 |
| 127 | 3-[(R)-2,2-difluorocyclopropyl]-2-(5-{[(3R,5R)-5-fluoro-1- methylpiperidin-3-yl]amino}-2H-pyrazolo[3,4-d][1,3]thiazol- 2-yl)-5-methylphenol or 3-[(S)-2,2-difluorocyclopropyl]-2- (5-{[(3R,5R)-5-fluoro-1-methylpiperidin-3-yl]amino}- 2H-pyrazolo[3,4-d][1,3]thiazol-2-yl)-5-methylphenol (tR1) | 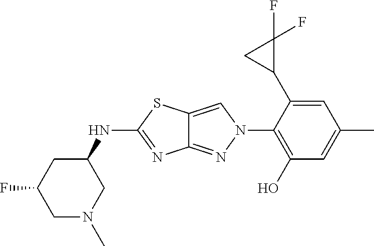 [SMILES: Cc1cc(O)c(-n2cc3sc(N[C@@H]4C[C@@H](F)CN(C)C4)nc3n2)c(C2CC2(F)F)c1] | 438.1 |
| 128 | 2-(5-{[1-(methanesulfonyl)piperidin-4-yl]amino}- 2H-pyrazolo[3,4-d][1,3]thiazol-2-yl)-3,5-dimethylphenol | 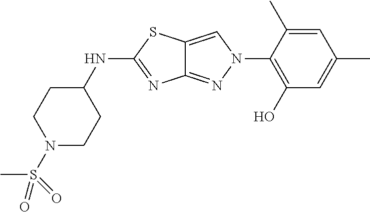 | 422.2 |
| 129 | 2-(5-{[1-(2,2-difluoroethyl)piperidin-4-yl]amino}- 2H-pyrazolo[3,4-d][1,3]thiazol-2-yl)-3,5-dimethylphenol | 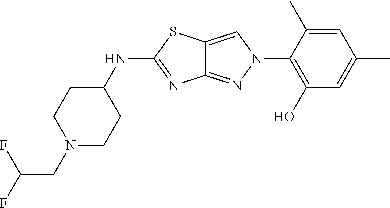 [SMILES: Cc1cc(C)c(-n2cc3sc(NC4CCN(CC(F)F)CC4)nc3n2)c(O)c1] | 408.2 |
| 130 | methyl 4-{[2-(2-hydroxy-4,6-dimethylphenyl)- 2H-pyrazolo[3,4-d][1,3]thiazol-5-yl]amino}piperidine-1- carboxylate | 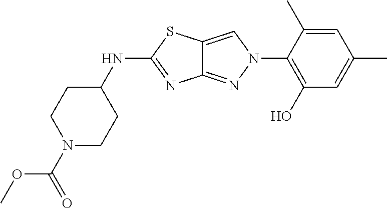 | 402.3 |

TABLE 1-14

| | | | |
|---|---|---|---|
| 131 | 4-{[2-(2-hydroxy-4,6-dimethylphenyl)-2H-pyrazolo[3,4-d][1,3] thiazol-5-yl]amino}-N-methylpiperidine-1-carboxamide | 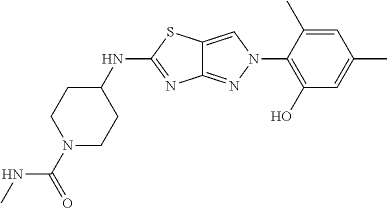 | 401.3 |
| 132 | 1-(4-{[2-(2-hydroxy-4,6-dimethylphenyl)- 2H-pyrazolo[3,4-d][1,3]thiazol-5-yl]amino}piperidin-1- yl)propan-1-one | 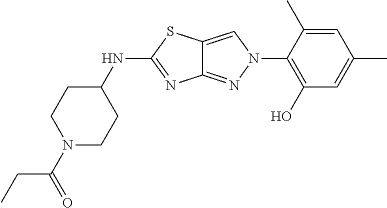 [SMILES: CCC(=O)N1CCC(Nc2nc3nn(-c4c(C)cc(C)cc4O)cc3s2)CC1] | 400.3 |
| 133 | 1-(4-{[2-(2-hydroxy-4,6-dimethylphenyl)- 2H-pyrazolo[3,4-d][1,3]thiazol-5-yl]amino}piperidin-1- yl)ethan-1-one | 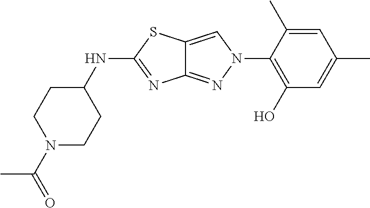 [SMILES: CC(=O)N1CCC(Nc2nc3nn(-c4c(C)cc(C)cc4O)cc3s2)CC1] | 386.2 |
| 134 | 2-(5-{[(3R,4R)-4-fluoro-1-methylpyrrolidin-3-yl] amino}-2H-pyrazolo[3,4-d][1,3]thiazol-2-yl)-5-methoxy- 3-methylphenol | 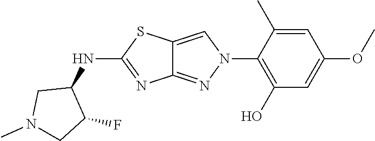 | 378.2 |
| 135 | 2-(5-{[(3S,4S)-4-fluoro-1-methylpyrrolidin-3-yl]amino}- 2H-pyrazolo[3,4-d][1,3]thiazol-2-yl)-5-methoxy-3- methylphenol | 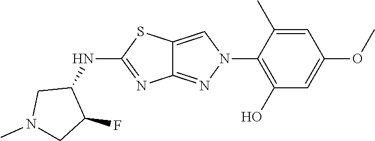 | 378.2 |
| 136 | (4S)-4-{[2-(2-hydroxy-4,6-dimethylphenyl)- 2H-pyrazolo[3,4-d][1,3]thiazol-5-yl]amino}-1- methylpiperidin-2-one | 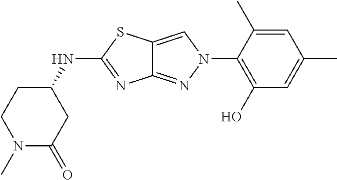 [SMILES: Cc1cc(C)c(-n2cc3sc(N[C@H]4CCN(C)C(=O)C4)nc3n2)c(O)c1] | 372.2 |
| 137 | (4R)-4-{[2-(2-hydroxy-4,6-dimethylphenyl)- 2H-pyrazolo[3,4-d][1,3]thiazol-5-yl]amino}-1-methylpiperidin-2-one | 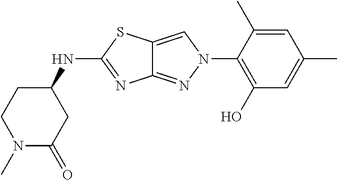 [SMILES: Cc1cc(C)c(-n2cc3sc(N[C@@H]4CCN(C)C(=O)C4)nc3n2)c(O)c1] | 372.2 |
| 138 | 2-(5-{[(1-fluorocyclobutyl)methyl]amino}- 2H-pyrazolo[3,4-d][1,3]thiazol-2-yl)-3,5-dimethylphenol | 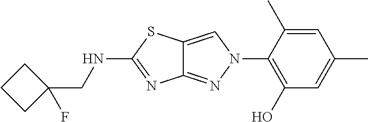 [SMILES: Cc1cc(C)c(-n2cc3sc(NCC4(F)CCC4)nc3n2)c(O)c1] | 347.1 |
| 139 | 2-{5-[(2,2-difluoropropyl)amino]-2H-pyrazolo[3,4-d][1,3] thiazol-2-yl}-3,5-dimethylphenol | 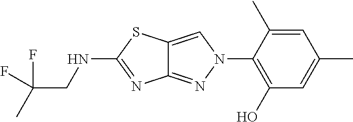 [SMILES: Cc1cc(C)c(-n2cc3sc(NCC(C)(F)F)nc3n2)c(O)c1] | 339.1 |
| 140 | 2-{5-[(3-hydroxybutan-2-yl)amino]-2H-pyrazolo[3,4-d] [1,3]thiazol-2-yl}-3,5-dimethylphenol | 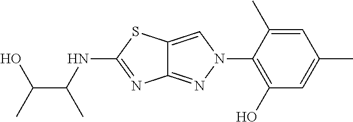 [SMILES: Cc1cc(C)c(-n2cc3sc(NC(C)C(C)O)nc3n2)c(O)c1] | 333.2 |

TABLE 1-15

| | | | |
|---|---|---|---|
| 141 | 3-(2,2-difluorocyclopropyl)-2-(5-{[(2R)-2-hydroxypropyl]amino}- 2H-pyrazolo[3,4-d][1,3]thiazol-2-yl)-5-methylphenol | 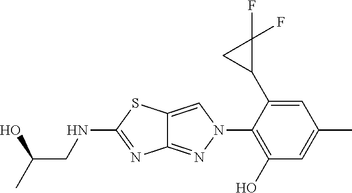 [SMILES: Cc1cc(O)c(-n2cc3sc(NC[C@@H](C)O)nc3n2)c(C2CC2(F)F)c1] | 381.1 |
| 142 | 2-(5-{[(3R,4S)-4-fluorooxolan-3-yl]amino}-2H-pyrazolo[3,4-d][1,3] thiazol-2-yl)-3,5-dimethylphenol or 2-(5-{[(3S,4R)-4-fluorooxolan-3-yl] amino}-2H-pyrazolo[3,4-d][1,3]thiazol-2-yl)-3,5-dimethylphenol (tR1) | 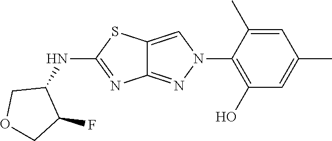 [SMILES: Cc1cc(C)c(-n2cc3sc(N[C@H]4COC[C@@H]4F)nc3n2)c(O)c1] | 349.1 |
| 143 | 2-(5-{[(3R,4S)-4-fluorooxolan-3-yl]amino}- 2H-pyrazolo[3,4-d][1,3]thiazol-2-yl)-3,5-dimethylphenol or 2-(5-{[(3S,4R)-4-fluorooxolan-3-yl]amino}- 2H-pyrazolo[3,4-d][1,3]thiazol-2-yl)-3,5-dimethylphenol (tR2) | 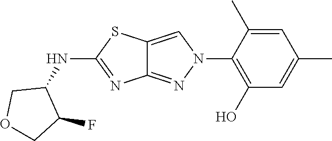 [SMILES: Cc1cc(C)c(-n2cc3sc(N[C@H]4COC[C@@H]4F)nc3n2)c(O)c1] | 349.1 |
| 144 | 2-(5-{[(3R,4R)-4-fluorooxolan-3-yl]amino}-2H-pyrazolo [3,4-d][1,3]thiazol-2-yl)-3,5-dimethylphenol or 2-(5- {[(3S,4S)-4-fluorooxolan-3-yl]amino}- 2H-pyrazolo[3,4-d][1,3]thiazol-2-yl)-3,5-dimethylphenol (tR1) | 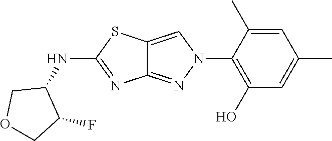 [SMILES: Cc1cc(C)c(-n2cc3sc(N[C@H]4COC[C@H]4F)nc3n2)c(O)c1] | 349.0 |
| 145 | 2-(5-{[(3R,4R)-4-fluorooxolan-3-yl]amino}- 2H-pyrazolo[3,4-d][1,3]thiazol-2-yl)-3,5-dimethylphenol or 2-(5-{[(3S,4S)-4-fluorooxolan-3-yl]amino}- 2H-pyrazolo[3,4-d][1,3]thiazol-2-yl)-3,5-dimethylphenol (tR2) | 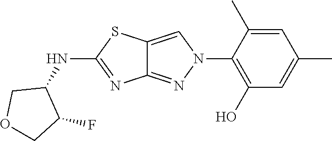 [SMILES: Cc1cc(C)c(-n2cc3sc(N[C@H]4COC[C@H]4F)nc3n2)c(O)c1] | 349.1 |
| 146 | 2-{5-[(2,2-difluoroethyl)amino]- 2H-pyrazolo[3,4-d][1,3]thiazol-2-yl}-3,5-dimethylphenol | 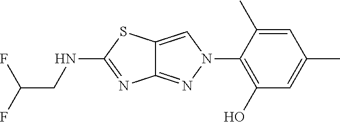 [SMILES: Cc1cc(C)c(-n2cc3sc(NCC(F)F)nc3n2)c(O)c1] | 325.1 |
| 147 | 2-(5-{[1-(difluoromethyl)cyclopropyl]amino}- 2H-pyrazolo[3,4-d][1,3]thiazol-2-yl)-3,5-dimethylphenol | 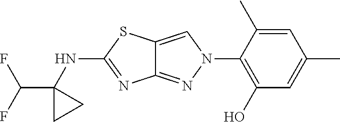 [SMILES: Cc1cc(C)c(-n2cc3sc(NC4(C(F)F)CC4)nc3n2)c(O)c1] | 351.1 |
| 148 | 3-(2,2-difluorocyclopropyl-2-{5-[(2-hydroxy-2- methylpropyl)amino]-2H-pyrazolo[3,4-d][1,3]thiazol- 2-yl}-5-methylphenol | 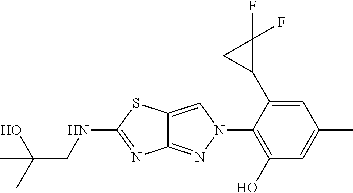 [SMILES: Cc1cc(O)c(-n2cc3sc(NCC(C)(C)O)nc3n2)c(C2CC2(F)F)c1] | 395.2 |
| 149 | 4-({2-[2-(2,2-difluorocyclopropyl)-6-hydroxy-4- methylphenyl]-2H-pyrazolo[3,4-d][1,3]thiazol-5- yl}amino)bicyclo[2.2.1]heptan-1-ol | 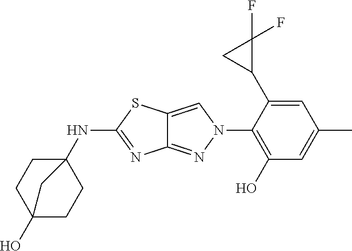 [SMILES: Cc1cc(O)c(-n2cc3sc(NC45CCC(O)(CC4)C5)nc3n2)c(C2CC2(F)F)c1] | 433.2 |
| 150 | 4-{[2-(2-ethyl-6-hydroxy-4-methylphenyl)- 2H-pyrazolo[3,4-d][1,3]thiazol-5-yl]amino}bicyclo[2.2.1]heptan-1-ol | 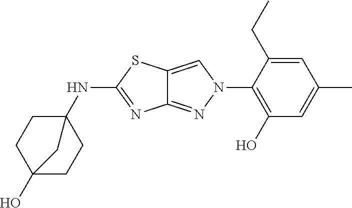 [SMILES: CCc1cc(C)cc(O)c1-n1cc2sc(NC34CCC(O)(CC3)C4)nc2n1] | 385.2 |

TABLE 1-16

| | | | |
|---|---|---|---|
| 151 | 4-{[2-(2-hydroxy-4,6-dimethylphenyl)- 2H-pyrazolo[3,4-d][1,3]thiazol-5-yl]amino} adamantan-1-ol | 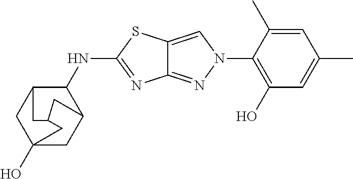 [SMILES: Cc1cc(C)c(-n2cc3sc(NC4C5CC6CC4CC(O)(C6)C5)nc3n2)c(O)c1] | 411.3 |
| 152 | 2-{5-[(2,2-difluoro-3-hydroxypropyl)amino]- 2H-pyrazolo[3,4-d][1,3]thiazol-2-yl}-3,5- dimethylphenol | 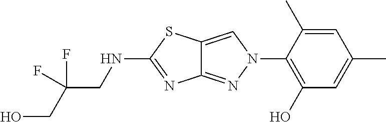 [SMILES: Cc1cc(C)c(-n2cc3sc(NCC(F)(F)CO)nc3n2)c(O)c1] | 355.0 |
| 153 | cyclopropyl (3R)-3-{[2-(2-hydroxy-4,6- dimethylphenyl)-2H-pyrazolo[3,4-d][1,3]thiazol-5-yl] amino}pyrrolidine-1-carboxylate | 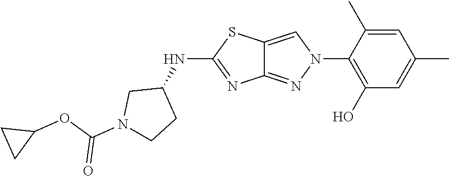 [SMILES: Cc1cc(C)c(-n2cc3sc(N[C@@H]4CCN(C(=O)OC5CC5)C4)nc3n2)c(O)c1] | 414.1 |
| 154 | cyclopropyl (3S)-3-{[2-(2-hydroxy-4,6-dimethylphenyl)- 2H-pyrazolo[3,4-d][1,3]thiazol-5-yl]amino}pyrrolidine-1- carboxylate | 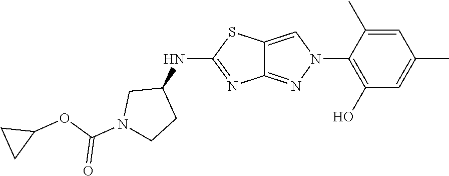 [SMILES: Cc1cc(C)c(-n2cc3sc(N[C@H]4CCN(C(=O)OC5CC5)C4)nc3n2)c(O)c1] | 414.2 |
| 155 | methyl (3R)-3-{[2-(2-hydroxy-4,6-dimethylphenyl)- 2H-pyrazolo[3,4-d][1,3]thiazol-5-yl]amino}pyrrolidine- 1-carboxylate | 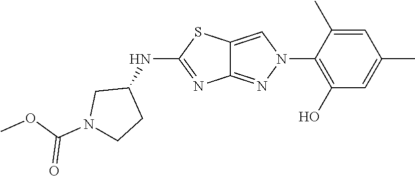 | 388.1 |
| 156 | methyl (3S)-3-{[2-(2-hydroxy-4,6-dimethylphenyl)- 2H-pyrazolo[3,4-d][1,3]thiazol-5-yl]amino}pyrrolidine- 1-carboxylate | 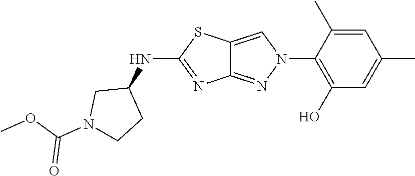 | 388.1 |
| 157 | 3,5-dimethyl-2-(5-{[(3R)-oxolan-3-yl]amino}- 2H-pyrazolo[3,4-d][1,3]thiazol-2-yl)phenol | 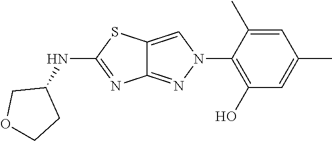 [SMILES: Cc1cc(C)c(-n2cc3sc(N[C@@H]4CCOC4)nc3n2)c(O)c1] | 331.1 |
| 158 | 3,5-dimethyl-2-(5-{[(3S)-oxolan-3-yl]amino}- 2H-pyrazolo[3,4-d][1,3]thiazol-2-yl)phenol | 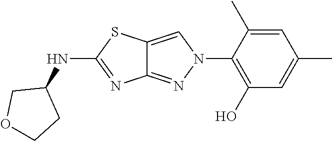 [SMILES: Cc1cc(C)c(-n2cc3sc(N[C@H]4CCOC4)nc3n2)c(O)c1] | 331.1 |
| 159 | 4-{[2-(2-hydroxy-4,6-dimethylphenyl)- 2H-pyrazolo[3,4-d][1,3]thiazol-5-yl]amino}oxolan-3-ol | 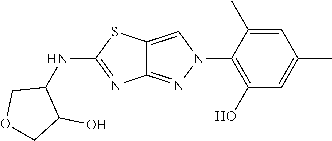 [SMILES: Cc1cc(C)c(-n2cc3sc(NC4COCC4O)nc3n2)c(O)c1] | 347.1 |
| 160 | 2-(5-{[(3S)-4,4-difluoro-1-methylpyrrolidin-3-yl]amino}- 2H-pyrazolo[3,4-d][1,3]thiazol-2-yl)-3,5-dimethylphenol | 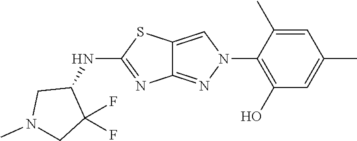 | 380.1 |

TABLE 1-17

| | | | |
|---|---|---|---|
| 161 | 3-({[2-(2-hydroxy-4,6-dimethylphenyl)-2H-pyrazolo [3,4-d][1,3]thiazol-5-yl]amino}methyl)oxetan-3-ol | 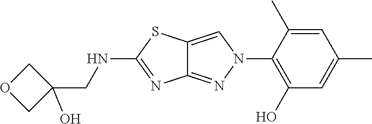 [SMILES: Cc1cc(C)c(-n2cc3sc(NCC4(O)COC4)nc3n2)c(O)c1] | 347.2 |
| 162 | 2-(5-{[(1R,2R)-2-hydroxycyclohexyl]amino}- 2H-pyrazolo[3,4-d][1,3]thiazol-2-yl)-3,5-dimethyl- phenol | 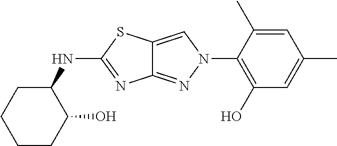 [SMILES: Cc1cc(C)c(-n2cc3sc(N[C@@H]4CCCC[C@H]4O)nc3n2)c(O)c1] | 359.2 |
| 163 | 3,5-dimethyl-2-{5-[(3,3,3-trifluoro-2-hydroxypropyl) amino]-2H-pyrazolo[3,4-d][1,3]thiazol-2-yl}phenol | 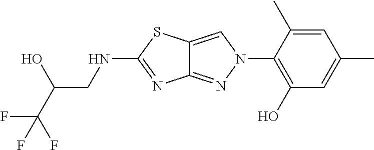 [SMILES: Cc1cc(C)c(-n2cc3sc(NCC(O)C(F)(F)F)nc3n2)c(O)c1] | 373.2 |
| 164 | 2-(5-{[2-hydroxy-2-(pyridin-2-yl)ethyl]amino}- 2H-pyrazolo[3,4-d][1,3]thiazol-2-yl)-3,5- dimethylphenol | 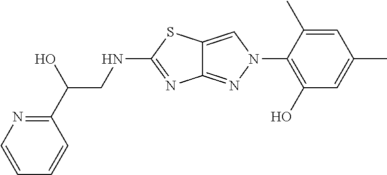 [SMILES: Cc1cc(C)c(-n2cc3sc(NCC(O)c4ccccn4)nc3n2)c(O)c1] | 382.2 |
| 165 | 1,5-anhydro-2,4-dideoxy-2-{[2-(2-hydroxy-4,6- dimethylphenyl)-2H-pyrazolo[3,4-d][1,3]thiazol-5- yl]amino}-D-threo-pentitol | 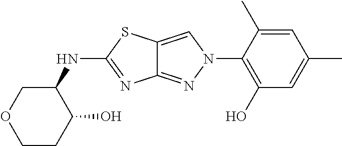 [SMILES: Cc1cc(C)c(-n2cc3sc(N[C@@H]4COCC[C@H]4O)nc3n2)c(O)c1] | 361.2 |
| 166 | 2-{5-[(2-hydroxy-3-methoxypropyl)amino]- 2H-pyrazolo[3,4-d][1,3]thiazol-2-yl}-3,5- dimethylphenol | 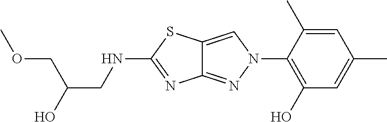 | 349.1 |
| 167 | 1,5-anhydro-2,3-dideoxy-3-{[2-(2-hydroxy-4,6- dimethylphenyl)-2H-pyrazolo[3,4-d][1,3]thiazol-5- yl]amino}-D-threo-pentitol | 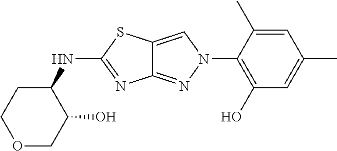 [SMILES: Cc1cc(C)c(-n2cc3sc(N[C@@H]4CCOC[C@H]4O)nc3n2)c(O)c1] | 361.1 |
| 168 | 2-{[2-(2-hydroxy-4,6-dimethylphenyl)- 2H-pyrazolo[3,4-d][1,3]thiazol-yl]amino}propane- 1,3-diol | 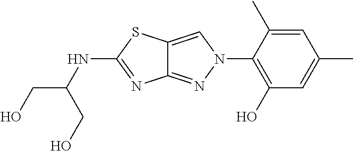 [SMILES: Cc1cc(C)c(-n2cc3sc(NC(CO)CO)nc3n2)c(O)c1] | 335.0 |
| 169 | 3-{[2-(2-hydroxy-4,6-dimethylphenyl)- 2H-pyrazolo[3,4-d][1,3]thiazol-5-yl]amino} bicyclo[1.1.1]pentan-1-ol | 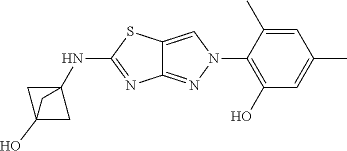 [SMILES: Cc1cc(C)c(-n2cc3sc(NC45CC(O)(C4)C5)nc3n2)c(O)c1] | 343.1 |
| 170 | 2-(5-{[3-(fluoromethyl)oxetan-3-yl]amino}- 2H-pyrazolo[3,4-d][1,3]thiazol-2-yl)-3,5- dimethylphenol | 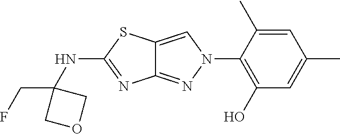 [SMILES: Cc1cc(C)c(-n2cc3sc(NC4(CF)COC4)nc3n2)c(O)c1] | 349.1 |

TABLE 1-18

| | | | |
|---|---|---|---|
| 171 | 2-{5-[(3,3-difluoro-2-hydroxypropyl)amino]- 2H-pyrazolo[3,4-d][1,3]thiazol-2-yl}-3,5- dimethylphenol | 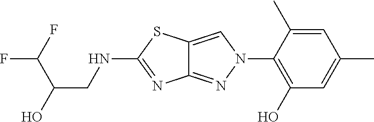 [SMILES: Cc1cc(C)c(-n2cc3sc(NCC(O)C(F)F)nc3n2)c(O)c1] | 355.1 |
| 172 | 2-(5-{[(2R)-2-(difluoromethoxy)propyl]amino}- 2H-pyrazolo[3,4-d][1,3]thiazol-2-yl)-3,5- dimethylphenol | 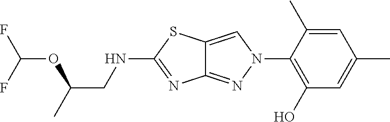 [SMILES: Cc1cc(C)c(-n2cc3sc(NC[C@@H](C)OC(F)F)nc3n2)c(O)c1] | 369.1 |
| 173 | 2-(5-{[3-(difluoromethoxy)bicyclo[1.1.1]pentan-1- yl]amino}-2H-pyrazolo[3,4-d][1,3]thiazol-2-yl)- 3,5-dimethylphenol | 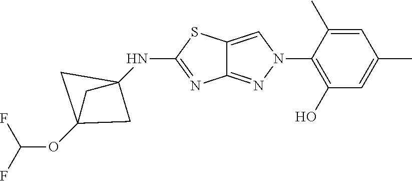 [SMILES: Cc1cc(C)c(-n2cc3sc(NC45CC(OC(F)F)(C4)C5)nc3n2)c(O)c1] | 393.2 |
| 174 | 2-(5-{[(3R,5R)-1-cyclopropyl-5-fluoropiperidin- 3-yl]amino}-2H-pyrazolo[3,4-d][1,3]thiazol-2- yl)-5-methoxy-3-methylphenol | 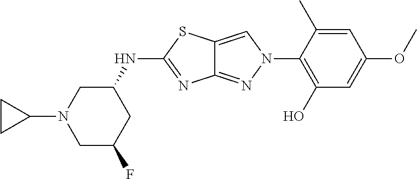 [SMILES: COc1cc(C)c(-n2cc3sc(N[C@@H]4C[C@@H](F)CN(C5CC5)C4)nc3n2)c(O)c1] | 418.3 |
| 175 | 3-(2,2-difluoroethyl)-2-(5-{[(3R,5R)-5-fluoro-1- methylpiperidin-3-yl]amino}-2H- pyrazolo[3,4-d][1,3]thiazol-2-yl)-5- methylphenol | 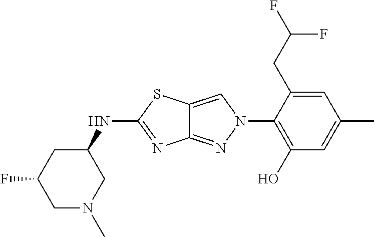 [SMILES: Cc1cc(O)c(-n2cc3sc(N[C@@H]4C[C@@H](F)CN(C)C4)nc3n2)c(CC(F)F)c1] | 426.4 |
| 176 | 3-[(difluoromethoxy)methyl]-2-(5-{[(3R,5R)-5- fluoro-1-methylpiperidin-3-yl]amino}- 2H-pyrazolo[3,4-d][1,3]thiazol-2-yl)-5- methylphenol | 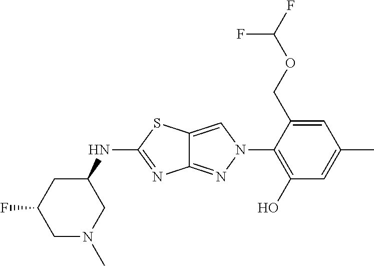 [SMILES: Cc1cc(O)c(-n2cc3sc(N[C@@H]4C[C@@H](F)CN(C)C4)nc3n2)c(COC(F)F)c1] | 442.3 |
| 177 | 2-(5-{[(3R,5R)-5-fluoro-1-methylpiperidin-3- yl]amino}-2H-pyrazolo[3,4-d][1,3]thiazol-2-yl)- 3-(methoxymethyl)-5-methylphenol | 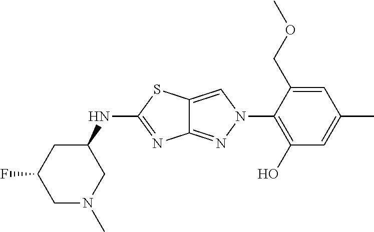 [SMILES: COCc1cc(C)cc(O)c1-n1cc2sc(N[C@@H]3C[C@@H](F)CN(C)C3)nc2n1] | 406.2 |
| 178 | 2-(5-{[(3S,5R)-5-fluoro-1-methylpiperidin-3- yl]amino}-2H-pyrazolo[3,4-d][1,3]thiazol-2-yl)- 3,5-dimethylphenol | 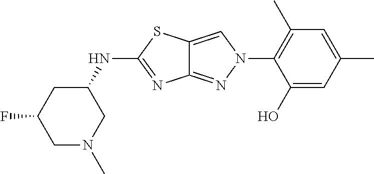 [SMILES: Cc1cc(C)c(-n2cc3sc(N[C@H]4C[C@@H](F)CN(C)C4)nc3n2)c(O)c1] | 376.2 |
| 179 | 2-(5-{[(3R,5S)-5-fluoro-1-methylpiperidin-3- yl]amino}-2H-pyrazolo[3,4-d][1,3]thiazol-2-yl)- 3,5-dimethylphenol | 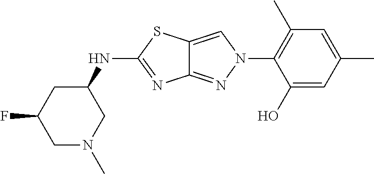 [SMILES: Cc1cc(C)c(-n2cc3sc(N[C@@H]4C[C@H](F)CN(C)C4)nc3n2)c(O)c1] | 376.2 |
| 180 | 2-(5-{[(3S,5S)-5-fluoro-1-methylpiperidin-3-yl] amino}-2H-pyrazolo[3,4-d][1,3]thiazol-2-yl)-3,5- dimethylphenol | 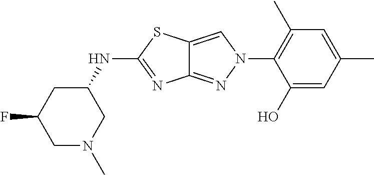 [SMILES: Cc1cc(C)c(-n2cc3sc(N[C@H]4C[C@H](F)CN(C)C4)nc3n2)c(O)c1] | 376.2 |
| 181 | 2-[5-(ethylamino)-2H-pyrazolo[3,4-d][1,3]thiazol- 2-yl]-3,5-dimethylphenol | 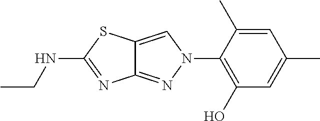 [SMILES: CCNc1nc2nn(-c3c(C)cc(C)cc3O)cc2s1] | 288.9 |

### Biological Activity

The biological activity of Compound (I) with respect to NLRP3 was determined using the following in vitro methods.

### IL-1β TR-FRET Assay (Reported as IC₅₀)

Monocytic THP-1 cells (ATCC: TIB-202) were maintained in accordance with the provider's instructions in RPMI media (Life Technologies, Cat #A10491-01); RPMI was supplemented with 10% heat inactivated fetal bovine serum (Corning, Cat #35-010-CV). The cells were differentiated into macrophages by the addition of 25 ng/mL IFN-γ (PeproTech, Cat #AF300-02-100UG) for 24 hours at 37 °C/5% CO₂. Media was exchanged with fresh media with no FBS, and the cells were treated with 50 ng/mL LPS (priming step) (LPS-EK: Invivogen, Cat #tlrl-peklps). The cells were plated at 240,000 cells per well in 384-well flat-bottom cell culture plates (FALCON, Cat #353962) and were incubated for 24 hours at 37 °C/5% CO₂. Compounds were serially diluted (half log dilutions) with DMSO and were finally diluted with Media with no FBS. The compounds were added to the cells in 384-well plates (added in 1:3) and then the plates were incubated for 30 minutes at 37 °C/5% CO₂. The NLRP3 inflammasome was activated with the addition of 20 mM ATP (Sigma Cat #A3377-25G) and the cells were incubated for 2 hours at 37 °C/5% CO₂. At the end of the incubation period, 30 μL supernatant was transferred to another 384-well plate and mixed on a plate shaker for 1 minutes. The supernatant was mixed with HTRF Antibody (Human IL1 beta kit, Cisbio, 62HIL1BPEH) in assay plates (Greiner Bio-One, Cat #784075) and the assay plates were incubated in shading box at room temperature for 16-24 hours. HTRF signal was measured by EnVision (Perkinelmer) in accordance with the manufacturer's instructions.

### TNF-α Assay (Reported as IC₅₀)

Monocytic THP-1 cells (ATCC: TIB-202) were maintained in accordance with the provider's instructions in RPMI media (Life Technologies, Cat #A10491-01); RPMI was supplemented with 10% heat inactivated fetal bovine serum (Hyclone Cat #SH30396.03). The cells were differentiated into macrophages by the addition of 25 ng/mL IFN-γ for 24 hours at 37 °C/5% CO₂. Media was exchanged with fresh media with no FBS. The cells were plated at 40,000 cells per well in 384-well flat-bottom cell culture plates (Costar 3764) containing compounds (added in 1:1000) in a 1:3.16 serial dilution series in DMSO and were incubated for 30 minutes at 37 °C/5% CO₂. The NF-κB pathway was activated with the addition of 50 ng/mL LPS and the cells were incubated for 3 hours at 37 °C/5% CO₂. At the end of the incubation period, supernatant (40 L) was removed, and IL-1β levels were monitored using an ELISA (Human TNF-α ELISA, R&D systems, Cat #DY210) according to the manufacturer's instructions.

### Data Interpretation

The IC₅₀ values were calculated from a plot of percentage of inhibition versus the inhibitor concentration by a logistics curve fit according to: Y=[Bottom+(Top−Bottom)]/(1+10\hat{ }[(Log IC₅₀−X)·Hill Slope], where Y is the % inhibition at the inhibitor concentration, X, “Bottom” is the lowest inhibition value, i.e., 0%, “Top” is the maximum inhibition value, i.e., 100%, and the “Hill Slope” describes the slope of the sigmoidal curve between the “Bottom” and “Top” values. The curve fitting was conducted with XLfit (ver. 5.5.0.5, ID Business Solutions Limited) or internally developed software.

Table 2 lists in vitro biological assay data (IL-1β and TNF-α) for the compounds shown in the examples. These assays are described in the section entitled Biological Activity, above.

TABLE 2
Biological Assay Data

| Example No. | IL-1β IC₅₀ (nM) | TNF-α IC₅₀ (nM) |
|---|---|---|
| 1 | 25 | >10000 |
| 2 | 1.6 | >30000 |
| 3 | 8.2 | >30000 |
| 4 | 3.6 | >30000 |
| 5 | 15 | >30000 |
| 6 | 980 | >10000 |
| 7 | 13 | >30000 |
| 8 | 290 | >30000 |
| 9 | 14 | >30000 |
| 10 | 46 | >10000 |
| 11 | 37 | >30000 |
| 12 | 3.6 | >10000 |
| 13 | 25 | >30000 |
| 14 | 20 | >30000 |
| 15 | 7000 | >30000 |
| 16 | 11 | >30000 |
| 17 | 110 | >30000 |
| 18 | 11 | >30000 |
| 19 | 5400 | >30000 |
| 20 | 9.2 | >30000 |
| 21 | 160 | >30000 |
| 22 | 1100 | >30000 |
| 23 | 210 | >30000 |
| 24 | 16 | >10000 |
| 25 | 350 | >10000 |
| 26 | 830 | >10000 |
| 27 | 620 | 6300 |
| 28 | 480 | >10000 |
| 29 | 540 | >10000 |
| 30 | 830 | >10000 |
| 31 | 650 | >30000 |
| 32 | 680 | >10000 |
| 33 | 1500 | >10000 |
| 34 | 150 | >10000 |
| 35 | 68 | >10000 |
| 36 | 34 | >30000 |
| 37 | 120 | >30000 |
| 38 | 440 | >30000 |
| 39 | 390 | >30000 |
| 40 | 1800 | >30000 |
| 41 | 1500 | >10000 |
| 42 | 14 | >30000 |
| 43 | 28 | >30000 |
| 44 | 240 | >10000 |
| 45 | 330 | >10000 |
| 46 | 330 | >10000 |
| 47 | 1200 | >10000 |
| 48 | 130 | >10000 |
| 49 | 1300 | >30000 |
| 50 | 12 | >30000 |
| 51 | 2.8 | >30000 |
| 52 | 200 | >10000 |
| 53 | 680 | >30000 |
| 54 | 1100 | >30000 |
| 55 | 1400 | >10000 |
| 56 | 230 | >30000 |
| 57 | 130 | >10000 |
| 58 | 2600 | >30000 |
| 59 | 23 | >30000 |
| 60 | 61 | >30000 |
| 61 | 2000 | >10000 |
| 62 | 120 | >10000 |
| 63 | 3400 | >10000 |
| 64 | 78 | >10000 |
| 65 | 8.3 | >30000 |
| 66 | 250 | >30000 |
| 67 | 950 | >30000 |
| 68 | 220 | >10000 |
| 69 | 250 | >10000 |
| 70 | 59 | >30000 |
| 71 | 43 | >30000 |
| 72 | 29 | >30000 |
| 73 | 860 | >30000 |
| 74 | 560 | >30000 |
| 75 | 16 | >30000 |
| 76 | 1600 | >30000 |
| 77 | 470 | >10000 |
| 78 | 4.3 | >10000 |
| 79 | 1100 | >30000 |
| 80 | 37 | >30000 |
| 81 | 3100 | >30000 |
| 82 | 49 | >30000 |
| 83 | 820 | >30000 |
| 84 | 380 | >30000 |
| 85 | 350 | >30000 |
| 86 | 300 | >30000 |
| 87 | 96 | >30000 |
| 88 | 440 | >30000 |
| 89 | 550 | >30000 |
| 90 | 540 | >10000 |
| 91 | 660 | >10000 |
| 92 | 270 | >30000 |
| 93 | 280 | >30000 |
| 94 | 31 | >30000 |
| 95 | 1300 | >30000 |
| 96 | 400 | >30000 |
| 97 | 260 | >10000 |
| 98 | 460 | >30000 |
| 99 | 250 | >30000 |
| 100 | 620 | >30000 |
| 101 | 420 | >30000 |
| 102 | 280 | >30000 |
| 103 | 810 | >30000 |
| 104 | 440 | >30000 |
| 105 | 310 | >30000 |
| 106 | 310 | >30000 |
| 107 | 75 | >30000 |
| 108 | 610 | >30000 |
| 109 | 590 | >30000 |
| 110 | 990 | >30000 |
| 111 | 810 | >30000 |
| 112 | 550 | >10000 |
| 113 | 510 | >30000 |
| 114 | 1000 | >10000 |
| 115 | 490 | >30000 |
| 116 | 1100 | >30000 |
| 117 | 1300 | >10000 |
| 118 | 780 | >30000 |
| 119 | 370 | >30000 |
| 120 | 720 | >30000 |
| 121 | 590 | >10000 |
| 122 | 580 | >30000 |
| 123 | 390 | >30000 |
| 124 | 340 | >30000 |
| 125 | 460 | >30000 |
| 126 | 3.8 | >30000 |
| 127 | 15 | >30000 |
| 128 | 1200 | >30000 |
| 129 | 2300 | >10000 |
| 130 | 1100 | >30000 |
| 131 | 580 | >30000 |
| 132 | 1000 | >30000 |
| 133 | 880 | >30000 |
| 134 | 1500 | >10000 |
| 135 | 90 | >10000 |
| 136 | 3300 | >30000 |
| 137 | 1100 | >30000 |
| 138 | 600 | >10000 |
| 139 | 280 | >10000 |
| 140 | 400 | >30000 |
| 141 | 59 | >30000 |
| 142 | 550 | >30000 |
| 143 | 430 | >30000 |
| 144 | 850 | >30000 |
| 145 | 440 | >30000 |
| 146 | 380 | >30000 |
| 147 | 840 | >10000 |
| 148 | 130 | >30000 |
| 149 | 66 | >30000 |
| 150 | 120 | >10000 |
| 151 | 6800 | >10000 |
| 152 | 280 | >30000 |
| 153 | 270 | >30000 |
| 154 | 1900 | >30000 |
| 155 | 340 | >30000 |
| 156 | 2000 | >10000 |
| 157 | 330 | >30000 |
| 158 | 660 | >30000 |
| 159 | 260 | >30000 |
| 160 | 1300 | >30000 |
| 161 | 16000 | >30000 |
| 162 | 450 | >30000 |
| 163 | 670 | >30000 |
| 164 | 1000 | >30000 |
| 165 | 1800 | >30000 |
| 166 | 760 | >30000 |
| 167 | 350 | >30000 |
| 168 | 370 | >30000 |
| 169 | 430 | >30000 |
| 170 | 2000 | >30000 |
| 171 | 160 | >10000 |
| 172 | 750 | >10000 |
| 173 | 3500 | >10000 |
| 174 | 110 | >30000 |
| 175 | 3.7 | >30000 |
| 176 | 22 | >30000 |
| 177 | 40 | >30000 |
| 178 | 8400 | >30000 |
| 179 | 94 | >30000 |
| 180 | 4200 | >30000 |
| 181 | 340 | >30000 |

The results showed that compounds of the present invention suppress the production of IL-1β. It was also confirmed that the compounds of the present invention show more selective to IL-1β than TNF-α. These indicate that the compounds inhibit the targeted NLRP3 inflammasome activation pathway with little or no interference of the NF-κB-dependent priming pathway. Considering the diversity of pro-inflammatory factors, often with opposing functions, specific inhibition of the NLRP3 inflammasome pathway is required to achieve the most desired outcome without impeding the tissue repair process.

### Formulation Example 1 (Production of Capsule)

| | |
|---|---|
| 1) compound of Example 1 | 30 mg |
| 2) crystalline cellulose | 10 mg |
| 3) lactose | 19 mg |
| 4) magnesium stearate | 1 mg |
| total | 60 mg |

1), 2), 3) and 4) are mixed and filled in a gelatin capsule.

### Formulation Example 2 (Production of Tablet)

| | |
|---|---|
| 1) compound of Example 1 | 30 g |
| 2) lactose | 50 g |
| 3) cornstarch | 15 g |
| 4) calcium carboxymethylcellulose | 44 g |
| 5) magnesium stearate | 1 g |
| 1000 tablets | 140 g in total |

The total amount of 1), 2), 3) and 30 g of 4) are kneaded with water, vacuum dried and sieved. The sieved powder is mixed with 14 g of 4) and 1 g of 5), and the mixture is punched by a tableting machine. In this way, 1000 tablets containing 30 mg of the compound of Example 1 per tablet are obtained.

As used in this specification and the appended claims, singular articles such as “a,” “an,” and “the,” may refer to a single object or to a plurality of objects unless the context clearly indicates otherwise. Thus, for example, reference to a composition containing “a compound” may include a single compound or two or more compounds. The above description is intended to be illustrative and not restrictive. Many embodiments will be apparent to those of skill in the art upon reading the above description. Therefore, the scope of the invention should be determined with reference to the appended claims and includes the full scope of equivalents to which such claims are entitled. The disclosures of all articles and references cited in the disclosure, including patents, patent applications and publications, are herein incorporated by reference in their entirety and for all purposes.

What is claimed is:

1. A compound of Formula (I), or a pharmaceutically acceptable salt thereof, 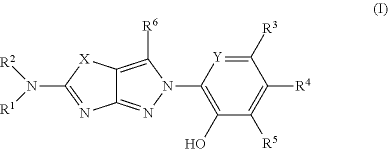
wherein
X is S or NR⁷;
Y is CR⁸ or N;
R¹ is an optionally substituted 4-membered heterocyclic group or 5- or 6-membered heterocyclic group, an optionally substituted C₁₋₆ alkyl group, or an optionally substituted C₃₋₁₀ cycloalkyl group;
R² is a hydrogen atom, or an optionally substituted C₁₋₃ alkyl group;
R³, R⁴ and R⁵ are each independently a hydrogen atom, a halogen atom, an optionally substituted C₁₋₆ alkyl group, an optionally substituted C₃₋₈ cycloalkyl group, or an optionally substituted C₁₋₆ alkoxy group;
R⁶ is a hydrogen atom, or an optionally substituted C₁₋₆ alkyl group;
R⁷ is a hydrogen atom, or an optionally substituted C₁₋₆ alkyl group; and
R⁸ is a hydrogen atom, a halogen atom, an optionally substituted C₁₋₆ alkyl group, an optionally substituted C₃₋₈ cycloalkyl, or an optionally substituted C₁₋₆ alkoxy group.

2. The compound or pharmaceutically acceptable salt thereof according to claim 1, wherein
X is S or NR⁷;
Y is CR⁸ or N;
R¹ is
(1) a 4-membered heterocyclic group or 5- or 6-membered heterocyclic group, wherein the heterocyclic groups are optionally substituted by 1 to 3 substituents selected from
(a) a halogen atom,
(b) an oxo group,
(c) a C₁₋₆ alkyl group optionally substituted by 1 to 3 substituents selected from
(i) a halogen atom, and
(ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
(i) a halogen atom,
(ii) a hydroxy group,
(iii) a C₁₋₆ alkoxy group, and
(iv) a C₃₋₈ cycloalkyl group,
(h) a C₃₋₈ cycloalkyl-carbonyl group optionally substituted by 1 to 3 substituents selected from
(i) a halogen atom,
(ii) a cyano group, and
(iii) a hydroxy group,
(i) a C₁₋₆ alkoxy-carbonyl group optionally substituted by 1 to 3 halogen atoms,
(j) a C₃₋₈ cycloalkoxy-carbonyl group
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group,
(2) a C₁₋₆ alkyl group optionally substituted by 1 to 3 substituents selected from
(a) a hydroxy group,
(b) a C₁₋₆ alkoxy group optionally substituted by 1 to 3 halogen atoms,
(c) a C₃₋₈ cycloalkyl group optionally substituted by 1 to 3 halogen atoms,
(d) a mono- or di-C₁₋₆ alkylamino group,
(e) a C₁₋₆ alkyl-carbonylamino group,
(f) a 3- to 8-membered non-aromatic heterocyclic group optionally substituted by a hydroxy group,
(g) a halogen atom, and
(h) a 5- to 14-membered aromatic heterocyclyl group, or
(3) a C₃₋₁₀ cycloalkyl group (the C₃₋₁₀ cycloalkyl group may be a spiro ring group or a bridged spiro ring group) optionally substituted by 1 to 3 substituents selected from
(a) a hydroxy group,
(b) a C₁₋₆ alkyl group optionally substituted by 1 to 3 halogen atoms, and
(c) a C₁₋₆ alkoxy group optionally substituted by 1 to 3 halogen atoms;
R² is
(1) a hydrogen atom, or
(2) a C₁₋₃ alkyl group optionally substituted by 1 to 3 C₁₋₆ alkoxy groups;
R³ is a hydrogen atom;
R⁴ is
(1) a hydrogen atom,
(2) a halogen atom,
(3) a C₁₋₆ alkyl group optionally substituted by 1 to 3 halogen atoms, or
(4) a C₁₋₆ alkoxy group optionally substituted by 1 to 3 halogen atoms;
R⁵ is a hydrogen atom;
R⁶ is a hydrogen atom or a C₁₋₆ alkyl group;
R⁷ is a C₁₋₆ alkyl group; and
R⁸ is
(1) a hydrogen atom,
(2) a halogen atom,
(3) a C₁₋₆ alkyl group optionally substituted by 1 to 3 substituents selected from (i) halogen atom and (ii) a C₁₋₆ alkoxy group optionally substituted by 1 to 3 halogen atoms,
(4) a C₃₋₈ cycloalkyl group optionally substituted by 1 to 3 halogen atoms, or
(5) a C₁₋₆ alkoxy group optionally substituted by 1 to 3 halogen atoms.

3. The compound or pharmaceutically acceptable salt thereof according to claim 1, wherein R² is a hydrogen atom.

4. The compound or pharmaceutically acceptable salt thereof according to claim 1, wherein X is NR.

5. The compound or pharmaceutically acceptable salt thereof according to claim 1, wherein X is NR⁷ and R⁷ is a C₁₋₆ alkyl group.

6. The compound or pharmaceutically acceptable salt thereof according to claim 1, wherein X is S.

7. The compound or pharmaceutically acceptable salt thereof according to claim 1, wherein R¹ is
(1) a 4-membered heterocyclic group or 5- or 6-membered heterocyclic group, wherein the heterocyclic groups are optionally substituted by 1 to 3 substituents selected from
(a) a halogen atom,
(b) an oxo group,
(c) a C₁₋₆ alkyl group optionally substituted by 1 to 3 substituents selected from
(i) a halogen atom, and
(ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
(i) a halogen atom,
(ii) a hydroxy group,
(iii) a C₁₋₆ alkoxy group, and
(iv) a C₃₋₈ cycloalkyl group,
(h) a C₃₋₈ cycloalkyl-carbonyl group optionally substituted by 1 to 3 substituents selected from
(i) a halogen atom,
(ii) a cyano group, and
(iii) a hydroxy group,
(i) a C₁₋₆ alkoxy-carbonyl group optionally substituted by 1 to 3 halogen atoms,
(j) a C₃₋₈ cycloalkoxy-carbonyl group
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group,
(2) a C₁₋₆ alkyl group optionally substituted by 1 to 3 substituents selected from
(a) a hydroxy group,
(b) a C₁₋₆ alkoxy group optionally substituted by 1 to 3 halogen atoms,
(c) a C₃₋₈ cycloalkyl group optionally substituted by 1 to 3 halogen atoms,
(d) a mono- or di-C₁₋₆ alkylamino group,
(e) a C₁₋₆ alkyl-carbonylamino group,
(f) a 3- to 8-membered non-aromatic heterocyclic group optionally substituted by a hydroxy group,
(g) a halogen atom, and
(h) a 5- to 14-membered aromatic heterocyclyl group, or
(3) a C₃₋₁₀ cycloalkyl group (the C₃₋₁₀ cycloalkyl group may be a spiro ring group or a bridged spiro ring group) optionally substituted by 1 to 3 substituents selected from
(a) a hydroxy group,
(b) a C₁₋₆ alkyl group optionally substituted by 1 to 3 halogen atoms, and
(c) a C₁₋₆ alkoxy group optionally substituted by 1 to 3 halogen atoms.

8. The compound or pharmaceutically acceptable salt thereof according to claim 1, wherein R¹ is a 4-membered heterocyclic group or 5- or 6-membered non-aromatic heterocyclic group, wherein the heterocyclic groups are optionally substituted by 1 to 3 substituents selected from
(a) a halogen atom,
(b) an oxo group,
(c) a C₁₋₆ alkyl group optionally substituted by 1 to 3 substituents selected from
(i) a halogen atom, and
(ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
(i) a halogen atom,
(ii) a hydroxy group,
(iii) a C₁₋₆ alkoxy group, and
(iv) a C₃₋₈ cycloalkyl group,
(h) a C₃₋₈ cycloalkyl-carbonyl group optionally substituted by 1 to 3 substituents selected from
(i) a halogen atom,
(ii) a cyano group, and
(iii) a hydroxy group,
(i) a C₁₋₆ alkoxy-carbonyl group optionally substituted by 1 to 3 halogen atoms,
(j) a C₃₋₈ cycloalkoxy-carbonyl group
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group.

9. The compound or pharmaceutically acceptable salt thereof according to claim 1, wherein R¹ is a 4-membered heterocyclic group or 5- or 6-membered non-aromatic heterocyclic group selected from pyrrolidinyl, piperidyl, oxetanyl, tetrahydrofuryl and tetrahydropyranyl, each of which is optionally substituted by 1 to 3 substituents selected from
(a) a halogen atom,
(b) an oxo group,
(c) a C₁₋₆ alkyl group optionally substituted by 1 to 3 substituents selected from
(i) a halogen atom, and
(ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
(i) a halogen atom,
(ii) a hydroxy group,
(iii) a C₁₋₆ alkoxy group, and
(iv) a C₃₋₈ cycloalkyl group,
(h) a C₃₋₈ cycloalkyl-carbonyl group optionally substituted by 1 to 3 substituents selected from
(i) a halogen atom,
(ii) a cyano group, and
(iii) a hydroxy group,
(i) a C₁₋₆ alkoxy-carbonyl group optionally substituted by 1 to 3 halogen atoms,
(j) a C₃₋₈ cycloalkoxy-carbonyl group,
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group.

10. The compound or pharmaceutically acceptable salt thereof according to claim 1, wherein Y is CR⁸ wherein R⁸ is
(1) a hydrogen atom,
(2) a halogen atom,
(3) a C₁₋₆ alkyl group optionally substituted by 1 to 3 substituents selected from (i) halogen atoms and (ii) a C₁₋₆ alkoxy group optionally substituted by 1 to 3 halogen atoms,
(4) a C₃₋₈ cycloalkyl group optionally substituted by 1 to 3 halogen atoms, or
(5) a C₁₋₆ alkoxy group optionally substituted by 1 to 3 halogen atoms.

11. The compound or pharmaceutically acceptable salt thereof according to claim 1, wherein R¹ is
(1) a 4-membered heterocyclic group or 5- or 6-membered heterocyclic group, wherein the heterocyclic groups are optionally substituted by 1 to 3 substituents selected from
(a) a halogen atom,
(b) an oxo group,
(c) a C₁₋₆ alkyl group optionally substituted by 1 to 3 substituents selected from
(i) a halogen atom, and
(ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
(i) a halogen atom,
(ii) a hydroxy group,
(iii) a C₁₋₆ alkoxy group, and
(iv) a C₃₋₈ cycloalkyl group,
(h) a C₃₋₈ cycloalkyl-carbonyl group optionally substituted by 1 to 3 substituents selected from
(i) a halogen atom,
(ii) a cyano group, and
(iii) a hydroxy group,
(i) a C₁₋₆ alkoxy-carbonyl group optionally substituted by 1 to 3 halogen atoms,
(j) a C₃₋₈ cycloalkoxy-carbonyl group,
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group,
(2) a C₁₋₆ alkyl group optionally substituted by 1 to 3 substituents selected from
(a) a hydroxy group,
(b) a C₁₋₆ alkoxy group optionally substituted by 1 to 3 halogen atoms,
(c) a C₃₋₈ cycloalkyl group optionally substituted by 1 to 3 halogen atoms,
(d) a mono- or di-C₁₋₆ alkylamino group,
(e) a C₁₋₆ alkyl-carbonylamino group,
(f) a 3- to 8-membered non-aromatic heterocyclic group optionally substituted by a hydroxy group,
(g) a halogen atom, and
(h) a 5- to 14-membered aromatic heterocyclyl group, or
(3) a C₃₋₁₀ cycloalkyl group (the C₃₋₁₀ cycloalkyl group may be a spiro ring group or a bridged spiro ring group) optionally substituted by 1 to 3 substituents selected from
(a) a hydroxy group,
(b) a C₁₋₆ alkyl group optionally substituted by 1 to 3 halogen atoms, and
(c) a C₁₋₆ alkoxy group optionally substituted by 1 to 3 halogen atoms; and
R² is a hydrogen atom.

12. The compound or pharmaceutically acceptable salt thereof according to claim 1, wherein
X is S;
R¹ is a 4-membered heterocyclic group or 5- or 6-membered non-aromatic heterocyclic group, wherein the heterocyclic groups are optionally substituted by 1 to 3 substituents selected from
(a) a halogen atom,
(b) an oxo group,
(c) a C₁₋₆ alkyl group optionally substituted by 1 to 3 substituents selected from
(i) a halogen atom, and
(ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
(i) a halogen atom,
(ii) a hydroxy group,
(iii) a C₁₋₆ alkoxy group, and
(iv) a C₃₋₈ cycloalkyl group,
(h) a C₃₋₈ cycloalkyl-carbonyl group optionally substituted by 1 to 3 substituents selected from
(i) a halogen atom,
(ii) a cyano group, and
(iii) a hydroxy group,
(i) a C₁₋₆ alkoxy-carbonyl group optionally substituted by 1 to 3 halogen atoms,
(j) a C₃₋₈ cycloalkoxy-carbonyl group,
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group; and
R² is a hydrogen atom.

13. The compound or pharmaceutically acceptable salt thereof according to claim 1, wherein
X is S;
R¹ is a 4-membered heterocyclic group or 5- or 6-membered non-aromatic heterocyclic group selected from pyrrolidinyl, piperidyl, oxetanyl, tetrahydrofuryl and tetrahydropyranyl, each of which is optionally substituted by 1 to 3 substituents selected from
(a) a halogen atom,
(b) an oxo group,
(c) a C₁₋₆ alkyl group optionally substituted by 1 to 3 substituents selected from
(i) a halogen atom, and
(ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
(i) a halogen atom,
(ii) a hydroxy group,
(iii) a C₁₋₆ alkoxy group, and
(iv) a C₃₋₈ cycloalkyl group,
(h) a C₃₋₈ cycloalkyl-carbonyl group optionally substituted by 1 to 3 substituents selected from
(i) a halogen atom,
(ii) a cyano group, and
(iii) a hydroxy group,
(i) a C₁₋₆ alkoxy-carbonyl group optionally substituted by 1 to 3 halogen atoms,
(j) a C₃₋₈ cycloalkoxy-carbonyl,
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group; and
R² is a hydrogen atom.

14. The compound or pharmaceutically acceptable salt thereof according to claim 1, wherein
X is S;
Y is CR⁸ wherein R⁸ is
(1) a hydrogen atom,
(2) a halogen atom,
(3) a C₁₋₆ alkyl group optionally substituted by 1 to 3 substituents selected from (i) halogen atoms and (ii) a C₁₋₆ alkoxy group optionally substituted by 1 to 3 halogen atoms,
(4) a C₃₋₈ cycloalkyl group optionally substituted by 1 to 3 halogen atoms, or
(5) a C₁₋₆ alkoxy group optionally substituted by 1 to 3 halogen atoms; and
R² is a hydrogen atom.

15. The compound or pharmaceutically acceptable salt thereof according to claim 1, wherein
R³ is a hydrogen atom;
R⁵ is a hydrogen atom; and
R⁶ is a hydrogen atom or a C₁₋₆ alkyl group.

16. The compound or pharmaceutically acceptable salt thereof according to claim 1, wherein
X is S;
R¹ is a 4-membered heterocyclic group or 5- or 6-membered non-aromatic heterocyclic group, wherein the heterocyclic groups are optionally substituted by 1 to 3 substituents selected from
(a) a halogen atom,
(b) an oxo group,
(c) a C₁₋₆ alkyl group optionally substituted by 1 to 3 substituents selected from
(i) a halogen atom, and
(ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
(i) a halogen atom,
(ii) a hydroxy group,
(iii) a C₁₋₆ alkoxy group, and
(iv) a C₃₋₈ cycloalkyl group,
(h) a C₃₋₈ cycloalkyl-carbonyl group optionally substituted by 1 to 3 substituents selected from
(i) a halogen atom,
(ii) a cyano group, and
(iii) a hydroxy group,
(i) a C₁₋₆ alkoxy-carbonyl group optionally substituted by 1 to 3 halogen atoms,
(j) a C₃₋₈ cycloalkoxy-carbonyl group,
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group;
R² is a hydrogen atom;
R³ is a hydrogen atom;
R⁵ is a hydrogen atom; and
R⁶ is a hydrogen atom or a C₁₋₆ alkyl group.

17. The compound or pharmaceutically acceptable salt thereof according to claim 1, wherein R² is a C₁₋₃ alkyl group optionally substituted by 1 to 3 C₁₋₆ alkoxy groups.

18. The compound or pharmaceutically acceptable salt thereof according to claim 1, wherein
X is S;
R¹ is
(1) a 4-membered heterocyclic group or 5- or 6-membered heterocyclic group, wherein the heterocyclic groups are optionally substituted by 1 to 3 substituents selected from
(a) a halogen atom,
(b) an oxo group,
(c) a C₁₋₆ alkyl group optionally substituted by 1 to 3 substituents selected from
(i) a halogen atom, and
(ii) a C₁₋₆ alkoxy group,
(d) a C₃₋₈ cycloalkyl group,
(e) a hydroxy group,
(f) a C₁₋₆ alkoxy group,
(g) a C₁₋₆ alkyl-carbonyl group optionally substituted by 1 to 3 substituents selected from
(i) a halogen atom,
(ii) a hydroxy group,
(iii) a C₁₋₆ alkoxy group, and
(iv) a C₃₋₈ cycloalkyl group,
(h) a C₃₋₈ cycloalkyl-carbonyl group optionally substituted by 1 to 3 substituents selected from
(i) a halogen atom,
(ii) a cyano group, and
(iii) a hydroxy group,
(i) a C₁₋₆ alkoxy-carbonyl group optionally substituted by 1 to 3 halogen atoms,
(j) a C₃₋₈ cycloalkoxy-carbonyl group,
(k) a C₇₋₁₆ aralkyloxy-carbonyl group,
(l) a mono- or di-C₁₋₆ alkyl-carbamoyl group,
(m) a C₁₋₆ alkylsulfonyl group,
(n) a C₃₋₈ cycloalkylsulfonyl group, and
(o) a 3- to 8-membered non-aromatic heterocyclic group,
(2) a C₁₋₆ alkyl group optionally substituted by 1 to 3 substituents selected from
(a) a hydroxy group,
(b) a C₁₋₆ alkoxy group optionally substituted by 1 to 3 halogen atoms,
(c) a C₃₋₈ cycloalkyl group optionally substituted by 1 to 3 halogen atoms,
(d) a mono- or di-C₁₋₆ alkylamino group,
(e) a C₁₋₆ alkyl-carbonylamino group,
(f) a 3- to 8-membered non-aromatic heterocyclic group optionally substituted by 1 hydroxy group,
(g) a halogen atom, and
(h) a 5- to 14-membered aromatic heterocyclyl group, or
(3) a C₃₋₁₀ cycloalkyl group (the C₃₋₁₀ cycloalkyl group may be a spiro ring group or a bridged spiro ring group) optionally substituted by 1 to 3 substituents selected from
(a) a hydroxy group,
(b) a C₁₋₆ alkyl group optionally substituted by 1 to 3 halogen atoms, and
(c) a C₁₋₆ alkoxy group optionally substituted by 1 to 3 halogen atoms; and
R² is a C₁₋₃ alkyl group optionally substituted by 1 to 3 C₁₋₆ alkoxy groups.

19. The compound or pharmaceutically acceptable salt thereof according to claim 1, wherein
X is S;
Y is CR⁸ wherein R⁸ is a C₁₋₆ alkyl group;
R¹ is a 6-membered nitrogen-containing non-aromatic heterocyclic group substituted by 1 or 2 substituents selected from
(a) a halogen atom, and
(b) a C₁₋₆ alkyl group;
R² is a hydrogen atom;
R³ is a hydrogen atom;
R⁴ is a C₁₋₆ alkyl group;
R⁵ is a hydrogen atom; and
R⁶ is a hydrogen atom.

20. The compound or pharmaceutically acceptable salt thereof according to claim 1, wherein
X is S;
Y is CR⁸ wherein R⁸ is a C₁₋₆ alkyl group;
R¹ is a piperidyl group substituted by 1 or 2 substituents selected from
(a) a halogen atom, and
(b) a C₁₋₂ alkyl group;
R² is a hydrogen atom;
R³ is a hydrogen atom;
R⁴ is a C₁₋₂ alkyl group;
R⁵ is a hydrogen atom; and
R⁶ is a hydrogen atom.

21. A compound selected from the title compounds of Examples 1 to 181, or a pharmaceutically acceptable salt thereof.

22. 2-(5-f{[(3R,5R)-5-fluoro-1-methylpiperidin-3-yl]amino}-2H-pyrazolo[3,4-d][1,3]thiazol-2-yl)-3,5-dimethylphenol or pharmaceutically acceptable salt thereof;
3,5-dimethyl-2-(5-{[(3R)-1-methylpiperidin-3-yl]amino}-2H-pyrazolo[3,4-d][1,3]thiazol-2-yl)phenol or pharmaceutically acceptable salt thereof; or
3-ethyl-2-(5-{[(3R,5R)-5-fluoro-1-methylpiperidin-3-yl]amino}-2H-pyrazolo[3,4-d][1,3]thiazol-2-yl)-5-methylphenol or pharmaceutically acceptable salt thereof.

23. A compound or pharmaceutically acceptable salt thereof as defined in claim 1 for use in treating a disease, disorder or condition associated with NLRP3.

24. A compound or pharmaceutically acceptable salt thereof as defined in claim 1 for use in treating a disease, disorder or condition associated with a heterozygous gain of function mutation in the NLRP3 gene.

25. A compound or pharmaceutically acceptable salt thereof as defined in claim 1 for use in treating a cryopyrin-associated periodic syndrome (CAPS).

26. The compound according to claim 25, wherein the cryopyrin-associated periodic syndrome is selected from neonatal-onset multisystem inflammatory disease (NOMID/CINCA), Muckle-Wells syndrome (MWS), and familial cold autoinflammatory syndrome (FCAS).

27. A method of treating a disease, disorder or condition in a subject, the method comprising administering to the subject an effective amount of a compound or pharmaceutically acceptable salt thereof as defined in claim 1, wherein the disease, disorder or condition is associated with NLRP3.

28. A method of treating a disease, disorder or condition in a subject, the method comprising administering to the subject an effective amount of a compound or pharmaceutically acceptable salt thereof as defined in claim 1, wherein the disease, disorder or condition is associated with a heterozygous gain of function mutation in the NLRP3 gene.

29. A method of treating a cryopyrin-associated periodic syndrome (CAPS) in a subject, the method comprising administering to the subject an effective amount of a compound or pharmaceutically acceptable salt thereof as defined in claim 1.

30. The method according to claim 29, wherein the cryopyrin-associated periodic syndrome is selected from neonatal-onset multisystem inflammatory disease (NOMID/CINCA), Muckle-Wells syndrome (MWS), and familial cold autoinflammatory syndrome (FCAS).

31. A method of treating a neurodegenerative disease, disorder or condition in a subject, the method comprising administering to the subject an effective amount of a compound or pharmaceutically acceptable salt thereof as defined in claim 1.

32. A method of treating Parkinson's disease, Alzheimer's disease, Huntington's disease, amyotrophic lateral sclerosis or prion disease in a subject, the method comprising administering to the subject an effective amount of a compound or pharmaceutically acceptable salt thereof as defined in claim 1.

33. A combination comprising a compound or pharmaceutically acceptable salt thereof as defined in claim 1, and at least one additional pharmacologically active agent.

34. The combination according to claim 33, wherein the additional pharmacologically active agent is selected from beta-secretase inhibitors, gamma-secretase inhibitors, HMG-CoA reductase inhibitors, nonsteroidal anti-inflammatory drugs, vitamin E, anti-amyloid antibodies, antidepressants, antipsychotics, anxiolytics, and anticonvulsants.

35. A medicament comprising a compound or pharmaceutically acceptable salt thereof as defined in claim 1.

36. The medicament according to claim 35, which is an agent for the treatment of disease, disorder or condition associated with NLRP3.

37. The medicament according to claim 35, which is an agent for the treatment of disease, disorder or condition associated with a heterozygous gain of function mutation in the NLRP3 gene.

38. The medicament according to claim 35, which is an agent for the treatment of a cryopyrin-associated periodic syndrome (CAPS).

39. The medicament according to claim 38, wherein the cryopyrin-associated periodic syndrome is selected from neonatal-onset multisystem inflammatory disease (NOMID/CINCA), Muckle-Wells syndrome (MWS), and familial cold autoinflammatory syndrome (FCAS).

40. Use of a compound or pharmaceutically acceptable salt thereof as defined in claim 1 for the manufacture of a medicament for the treatment of disease, disorder or condition associated with NLRP3.

41. Use of a compound or pharmaceutically acceptable salt thereof as defined in claim 1 for the manufacture of a medicament for the treatment of disease, disorder or condition associated with a heterozygous gain of function mutation in the NLRP3 gene.

42. Use of a compound or pharmaceutically acceptable salt thereof as defined in claim 1 for the manufacture of a medicament for the treatment of a cryopyrin-associated periodic syndrome (CAPS).

43. The use according to claim 42 wherein the cryopyrin-associated periodic syndrome is selected from neonatal-onset multisystem inflammatory disease (NOMID/CINCA), Muckle-Wells syndrome (MWS), and familial cold autoinflammatory syndrome (FCAS).
